(19) United States
(12) Patent Application Publication
(10) Pub. No.: US 2026/0363847 A1
(43) Pub. Date: Nov. 19, 2026

(54) SYSTEMS AND METHODS FOR COVERING CARDIAC DEVICES

(71) Applicant: Nyra Medical, Inc., Atlanta, GA (US)

(72) Inventors: Kirthana Sreerangathama SURESH, Atlanta, GA (US); Sinatha SOK, Minneapolis, MN (US); Sai Muralidhar PADALA, Decatur, GA (US)

(21) Appl. No.: 19/675,304

(22) Filed: May 12, 2026

Related U.S. Application Data
(60) Provisional application No. 63/805,113, filed on May 13, 2025.

(51) Int. Cl. A61F 2/24

(52) U.S. Cl. CPC A61F 2/24; A61F 2220/0075

(57) ABSTRACT

Embodiments described herein include an implant comprising an implant frame configured to be disposed around a portion of a native leaflet of a heart valve of a patient; a suture configured to be wrapped around a first portion of the implant frame to cover a surface of the first portion of the implant frame; and a cover configured to be disposed around a second portion of the implant frame to form a coaptation surface of the implant, the coaptation surface configured to cause coaptation between the native leaflet and an opposing native leaflet in the heart valve.

### CROSS-REFERENCE TO RELATED APPLICATIONS

This application claims priority to and the benefit of U.S. Provisional Application No. 63/805,113, filed May 13, 2025, entitled “Systems and Methods for Covering Cardiac Devices,” the disclosure of which is hereby incorporated by reference herein in its entirety.

### TECHNICAL FIELD

Embodiments described herein relate to systems and methods for covering a cardiac implant. In particular, embodiments described herein relate to covers for an implant for reducing heart valve regurgitation.

### BACKGROUND

Heart valve regurgitation, particularly functional mitral regurgitation (FMR), is a common heart valve lesion seen in patients surviving with heart failure (HF) or chronic atrial fibrillation (AFib), and it has been shown to worsen the progression of HF and AFib and increase morbidity and mortality of patients. Transcatheter approaches for delivering implants to the heart valves (e.g., the mitral valve) to correct FMR are useful approaches in that they are relatively lower risk, than more invasive surgery. Visibility of the implant on clinical imaging modalities, integration, compatibility, and functionality of the cardiac implant can hinge on proper coverings of the implant frame. Embodiments described herein provide methods for covering cardiac implants to ensure effective treatment and long-term durability of the device.

### SUMMARY

In some embodiments, an implant includes an implant frame configured to be disposed around a portion of a native leaflet of a heart valve of a patient; a suture configured to be wrapped around a first portion of the implant frame to cover a surface of the first portion of the implant frame; and a cover configured to be disposed around a second portion of the implant frame to form a coaptation surface of the implant, the coaptation surface configured to cause coaptation between the native leaflet and an opposing native leaflet in the heart valve.

In some embodiments, an implant includes an implant frame including: a first end portion configured to be disposed around a portion of a native leaflet to couple the implant to a heart valve of a patient; and a second end portion configured to extend into a gap of the heart valve; and a plurality of support members extending from the first end portion of the implant frame to the second end portion of the implant frame. The implant further includes a cover configured to form a coaptation surface configured to contact an opposing native leaflet of the heart valve, a portion of the cover configured to be disposed around the second end portion of the implant frame, the cover further including a pillow formed on an outer surface of the portion of the cover and extending from the second end portion of the implant frame to increase an area of the coaptation surface formed by the portion of the cover.

In some embodiments, an implant includes an implant frame including a first end portion including a first arm and a second arm configured to clamp a portion of a native leaflet to couple the implant to a heart valve of a patient; and a second end portion configured to extend into a gap of the heart valve, the second end portion including a central member coupled to the first arm and the second arm; and a plurality of support members extending from the first end portion of the implant frame distally beyond the central member; and a cover configured to be disposed around the plurality of support members to form a coaptation surface configured to contact an opposing native leaflet of the heart valve.

In some embodiments, an implant includes an implant frame including a first arm and a second arm configured to clamp a portion of a native leaflet of a heart valve of a patient therebetween, and a central portion between the first arm and the second arm; a suture configured to be wrapped around a portion of the implant frame to cover a surface of the portion of the implant frame; and a cover configured to be coupled to the implant frame, a first portion of the cover configured to be disposed around the central portion, a second portion of the cover separate from the first portion of the cover configured to be disposed around the second arm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B are schematic diagrams of a cardiac implant including a cover, according to embodiments.

FIG. 2 is a flow chart of an example method for covering a cardiac implant with a cover, according to an embodiment.

FIGS. 3A-3D are flow charts of example methods for covering different portions of a cardiac implant with a cover, according to an embodiment.

FIG. 4A is a front view of a cardiac implant and FIG. 4B is a back view of the cardiac implant, according to embodiments. FIG. 4C is a perspective view of the implant. FIG. 4D is a close-up view of support members of the implant, according to embodiments.

FIGS. 5A-5B show front and back views of a cardiac implant illustrating a portion of a cardiac implant for a user to hold while coupling a cover to the cardiac implant, according to embodiments.

FIGS. 6A-15B show images of steps of a method of wrapping a suture around a portion of an implant frame, according to an embodiment.

FIGS. 16-28B show images of steps of forming a ventricular sock and coupling the ventricular sock to the implant frame, according to an embodiment.

FIGS. 29-45B show images of steps of forming a central cover and coupling the central cover to a portion of the implant frame, according to an embodiment.

FIGS. 46A-52C show images of steps of forming a pillow and securing the pillow to the central cover coupled to the implant frame, according to an embodiment.

FIG. 53 shows an alignment of the pillow relative to support members of the implant frame, according to an embodiment.

FIG. 54A shows an implant including a cover coupled to an implant holder in a closed configuration, according to an embodiment. FIG. 54B shows the implant including the cover coupled to the implant holder in an open configuration, according to an embodiment. FIG. 54C shows the implant loosened away from the implant holder, according to embodiments.

FIG. 55 shows an implant covered with a cover and a suture according to a different method, according to embodiments.

FIGS. 56A-58B show close up images of stress on the support members of the implant frame that can arise from certain methods of covering the implant frame.

FIGS. 59-60E show images of a method of wrapping a suture around a spine of the implant, according to embodiments.

FIGS. 60F-67B show a method of forming a central cover and at least partially securing the central cover to the implant, according to embodiments.

FIGS. 68A-69B show a method of wrapping an atrial arm of an implant with a suture, according to embodiments.

FIGS. 70A-75 show a method of securing the central cover to the implant, according to embodiments.

FIGS. 76A-79D show different shapes of central covers of an implant, according to embodiments.

### DETAILED DESCRIPTION

Heart valve regurgitation has been shown to worsen the progression of heart failure (HF) and increase the mortality rate of patients. Most HF patients are deemed as too high-risk for traditional open-heart surgeries, due to their poor health condition and the risks associated with them. Therefore, transcatheter technologies to repair or replace the valve are in need for such patients. Current technologies fail to adequately treat FMR and disturb the native valve dynamics resulting in poor long-term durability. Furthermore, long-term durability, functionality, and compatibility of the implants can be dependent on proper coverings of the implant frame.

Disclosed herein are devices, systems, and methods for reducing heart valve regurgitation. Generally, the devices (e.g., implants) described herein may be configured to couple to a heart valve such that the heart valve may be altered to treat a patient's cardiac condition, such as heart valve regurgitation. The devices described herein generally comprise implantable devices configured to couple to cardiac tissue, such as one or more of a valve leaflet, chordae tendineae, and an annulus of a heart valve, to reduce or eliminate heart valve regurgitation. The devices may comprise an attachment section and a coaptation section. The attachment section may be configured to couple the implant to tissue, and the coaptation section may be configured (e.g., alone or in combination with the attachment section) to enhance and/or alter one or more physical characteristics of the heart valve.

In some variations, the one or more altered physical characteristics of the heart valve may reduce or eliminate a gap between leaflets, thereby reducing or eliminating valve regurgitation. For example, the devices described herein may alter one or more physical characteristics of a valve in a manner that increases a coaptation surface area for the valve leaflets. That is, the devices described herein may couple to a leaflet of a valve such that a length, thickness, and/or width of that leaflet is modified (e.g., increased). The modified length, width, and/or thickness of the leaflet may provide for a modified coaptation surface (e.g., increased coaptation surface area) that decreases or eliminates regurgitation. For instance, the devices described herein may protrude or otherwise extend from a surface of a heart valve leaflet such that another leaflet may coapt onto a portion of the device during systole, which may re-establish unidirectional blood flow through the valve. In some embodiments, the devices described herein may couple to the valve such that leaflet movement is modified in a manner that reduces or eliminates regurgitation.

The devices described herein may be deformable such that the devices may non-destructively (e.g., atraumatically) affect the structure or function of the heart valve and/or may receive a portion of the heart valve (e.g., a leaflet) via coaptation. For example, the devices may releasably attach to heart valve tissue (e.g., native leaflets, chordae tendineae) via a compressive force and/or friction force that may not damage the tissue. The compressive force may be applied by an attachment section comprising a first segment or arm and a second segment or arm of the implant. In some configurations, at least a portion of each of the first and second segments or arms may be coplanar with one another, which may assist in maintaining the position of the device relative to the heart valve. The central member may define a portion of a coaptation section, and the coaptation section may further include one or more support members. The support members may extend from the first segment or arm and may assist in defining a volume within the coaptation section. At least a portion of the coaptation section (e.g., at least a portion of the central member, one or more support members or a portion thereof) may be deflectable and/or deformable upon contact by a native leaflet. For example, one or more support members may deflect and/or deform relative to one or more additional support members. By virtue of the deflection and/or deformation of one or more of the support members (e.g., two, three, four, or more), the coaptation section volume at least partially defined by the support members may change (e.g., may decrease) upon contact by a native leaflet, and may be restored to its pre-contact volume upon loss of contact with the native leaflet.

Embodiments described herein can include coverings of the implant configured to not only maintain full functionality of an attachment section of the implant but also improve functionality of a coaptation section of the implant by including coverings that ensure coaptation of the leaflets during systole. In some embodiments, the covering of the implant may also improve integration of the implant on the leaflet as well as reduce damage to tissue of the heart valve. In some embodiments, the covering of the implant can enhance visibility of the implant underneath imaging modalities such as ultrasound. For example, the implant including the covering may have higher visibility (i.e., higher contrast from background) than a bare metal frame implant.

In some embodiments, the implant may include a cover configured to be disposed around at least a portion of the implant frame. In some embodiments, the cover may include one or more portions of fabric configured to be coupled to the implant frame via a suture. The suture may secure the cover to the implant to limit and/or prevent sliding and/or movement of the fabric relative to the implant frame. In some embodiments, a single suture may be wrapped around a portion of the implant frame to form the cover on the implant frame. In some embodiments, the cover may include one or more sections of fused (e.g., cauterized, glued, melted) fabric to create a pocket, then the edges of the pocket can be sutured together along the fused section. Therefore, fusing the edges may set or maintain a shape of the pocket to prevent puckering or sliding of the fabric when the fabric is sutured, thereby improving ease of seam creating. In some embodiments, reducing bunching or puckering of the fabric may reduce a thickness of the cover. In some embodiments, one or more sections may be fused to reduce a thickness (i.e., a profile) of the cover, thereby ensuring the attachment section of the implant can close (e.g., return to or near a coplanar state) to apply a desired compressive force to the leaflet of the heart valve. In some embodiments, a portion of the cover configured to be disposed over one of the arms or segments of the implant may be configured to have a low thickness or low profile to allow the first and second arms or segments to close and apply the desired compressive force, while also covering sharp edges of the implant frame to prevent damage to the heart valve tissue. In some embodiments, the covering may be configured to allow friction elements to extend from the implant frame through the covering such that the friction elements can grip the heart valve tissue to which the implant is coupled.

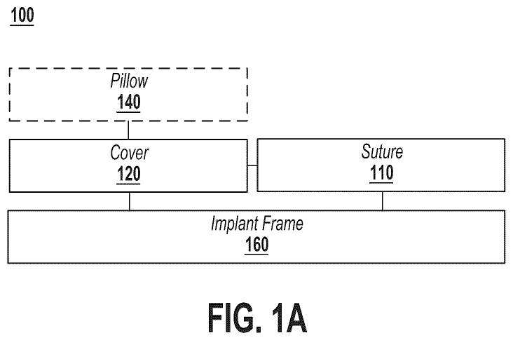

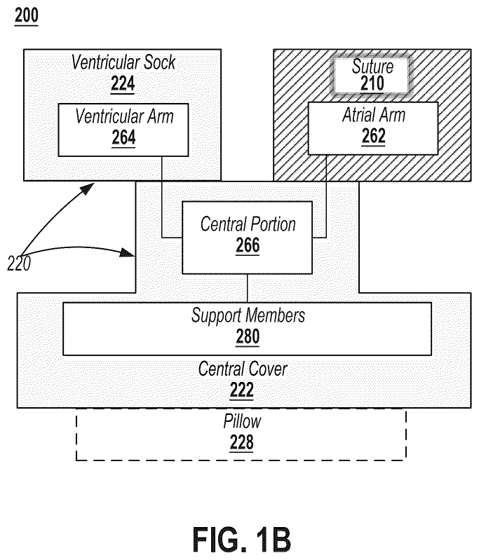

FIG. 1A is a schematic diagram of a cardiac implant (hereinafter, “implant”) 100 including an implant frame 160 and a cover 120 coupled to the implant frame 160. In some embodiments, the cover 120 can be coupled to the implant frame 160 via a suture 110 (e.g., a suture anchor), according to embodiments. The implant 100 may include one or more sections configured to couple to one or more portions of a heart valve, such that the implant may reduce or eliminate heart valve regurgitation. In some embodiments, the implant 100 may have a coaptation section comprising a 3-dimensional shape and an attachment section configured to couple the implant 100 to a heart valve (e.g., mitral, tricuspid, pulmonic, aortic) at a treatment site thereof. The treatment site may correspond to a portion of the heart valve that contributes to a cardiac condition, such as heart valve regurgitation during systole. For example, the treatment site may be located along the anterior leaflet of the mitral valve (e.g., A1, A2, A3), posterior leaflet of the mitral valve (e.g., P1, P2, P3), chordae tendineae of the mitral valve, anterior leaflet (i.e., cusp) of the tricuspid valve, posterior leaflet (i.e., cusp) of the tricuspid valve, septal leaflet (i.e., cusp) of the tricuspid valve, chordae tendineae of the tricuspid valve, left coronary leaflet (i.e., cusp) of the aortic valve, right coronary leaflet (i.e., cusp) of the aortic valve, non-coronary leaflet (i.e., cusp) of the aortic valve, left leaflet (i.e., cusp) of the pulmonary valve, right leaflet (i.e., cusp) of the pulmonary valve, or anterior leaflet (i.e., cusp) of the pulmonary valve.

The coaptation section of the implant may be configured (alone or in combination with the attachment section) to enhance and/or alter a physical characteristic of the native leaflet (e.g., a first native leaflet). In some variations, the coaptation section may be configured to provide a coaptation surface for another native leaflet of the heart valve (e.g., a second native leaflet). For example, the coaptation section may be configured to provide a contact surface for non-destructively receiving the second native leaflet during systole, which may reduce or eliminate heart valve regurgitation (e.g. reduce or close a gap between leaflets). At least a portion of the coaptation section may deflect upon coaptation by the native leaflet. The coaptation section may define a volume, and a portion of the first native leaflet may be positioned within the volume when the device is coupled to the first native leaflet.

The attachment section of the implant may include two segments (e.g., planar segments) or arms configured to couple to portions of a heart valve thereby coupling the implant to one or more of a native leaflet, and chordae tendineae. For example, the attachment section may comprise a first segment or arm and at least a portion of a second segment or arm. The first and second segments or arms may be biased towards one another such that the first and second segments or arms hold (e.g., compress) heart valve tissue (e.g., posterior or anterior leaflet of the mitral valve) received therebetween. The first and second segments or arms may be configured to securely attach to the heart valve, such that the implant may not come loose after implantation.

The implant may include central member (or spine) from which the first and second segments or arms may each extend. The first segment or arm may extend from a first portion of the central member and the second segment or arm may extend from a second, different portion of the central member. In some embodiments, the first and second segments or arms may extend from opposite ends of the central member. The central member may be shaped (e.g., curved) to bias or assist in biasing the first and second segments or arms towards one another. The biasing force provided by the central member may be sufficient to maintain the first and second segments or arms in place when coupled to heart valve tissue.

The implant may further include one or more support members configured to provide a surface for coaptation by a native leaflet, such as by supporting a cover. The one or more support members supporting the cover may be referred to as a first portion of the coaptation section. The coaptation section may further comprise a second portion extending from a distal end of the first portion. The second portion may be devoid of support members. In some embodiments, the support members may extend from the central member. In some embodiments, the support members may extend from the first segment or arm and may be configured to elastically deform with each interaction with the opposing leaflet, which may contribute to the durability and effectiveness of the implant. The support members may extend from the first segment or arm to define a larger volume of the coaptation section relative to a variation in which the support members extend from the central member.

The support members may comprise a free end such that the support members may move (e.g., deflect, extend, contract) relative to other portions of the implant. For example, the support members may deflect when a leaflet applies a force to the coaptation section (e.g., during systole) and elastically return to their previous configuration when the leaflet is not applying the force to the coaptation section (e.g., during diastole).

In some embodiments, the implant 100 may include the cover 120 disposed around at least a portion of the implant frame 160. In some embodiments, the suture 110 may be wrapped around at least a portion of the implant frame 160. In some embodiments, the suture 110 may act as an anchor to secure the cover 120 to the implant frame 160. For example, one or more stitches may pass through the cover 120 and one or more wraps or loops of the suture 110 to secure the cover 120 to the implant frame 160. The suture 110 may prevent the cover 120 from slipping relative to the implant frame 160. In some embodiments, the suture 110 may include a first end portion configured to be wrapped around a first portion of the implant frame and a second end portion configured to couple a portion of the cover 120 to a second portion of the implant frame 160. In some embodiments, the cover 120 may be configured to be stitched to the suture 110 to secure the cover 120 to the implant frame 160. In some embodiments, the implant 100 may include no more than one suture wrapped around the implant frame 160. In other words, only one suture 110 may be wrapped around the implant frame 160 to act as a cover for a portion of the implant frame 160 and/or as the anchor for the cover 120. Alternatively, the implant 100 may include a plurality of sutures 110 wrapped around the implant frame 160 to act as an anchor for the cover 120. In some embodiments, the suture 110 may include any suitable biocompatible material such as, for example, ultra-high molecular weight polyethylene. In some embodiments, the suture 110 and the cover 120 may include different materials.

In some embodiments, the cover 120 may include one or more portions or sections. In some embodiments, the one or more portions or sections may be formed from laser cutting and/or die cutting fabric into a predetermined shape (e.g., with predetermined dimensions corresponding to the implant frame 160). In some embodiments, at least some portions of the cover 120 may be cut and fused simultaneously. For example, a device may be used to cut the fabric and cauterize the fabric to form one or more socks or pockets configured to be disposed over the implant frame 160. In some embodiments, a first portion (e.g., a central cover) of the cover 120 may be configured to be disposed around a first portion of the implant 160 and a second portion of the cover 120 (e.g., a ventricular sock) separate from the first portion may be configured to be disposed around a second portion of the implant 160. In some embodiments, the second portion of the cover 120 may be formed with cauterized edges such that the second portion of the cover 120 has a thickness (e.g., a height the cover 120 extends from a surface of the implant frame 160) lower than a predetermined threshold (e.g., the second portion of the cover 120 has a low profile). In some embodiments, the cover 120 may optionally include and/or be coupled to a pillow 140 (e.g., an extension), as indicated with dashed lines. Dashed lines can be used herein to indicate a component or step is optional.

In some embodiments, the pillow 140 may form at least a portion of the coaptation surface of the implant 100 when the implant 100 is coupled to the native leaflet of the heart valve of the patient. In some embodiments, the first portion of the cover 120 may form a coaptation surface, and the pillow 140 may extend the coaptation surface of the implant to ensure coaptation occurs. In some embodiments, the first portion of the cover 120 may form a first coaptation surface, and the pillow 140 may form a second coaptation surface extending from or adding to the first coaptation surface. In some embodiments, the pillow 140 may provide a larger coaptation surface to enable sufficient coaptation over a plurality of different placements of the implant between the two leaflets. Therefore, the pillow 140 may enable sufficient coaptation with less precise positioning of the implant by a surgeon. In some embodiments, the pillow 140 may be disposed at a distal end of the implant frame 160 such that the pillow 140 extends into an opening of the heart valve when the implant 100 is coupled to the native leaflet of the native heart valve. In some embodiments, the cover 120 and/or the pillow 140 may be formed from a fabric (e.g., a biocompatible fabric). In some embodiments, the cover 120 and/or the pillow 140 may protect tissue in the heart from damage from the implant frame 160. For example, the implant frame 160 may include a metal (or any suitable rigid material), and the cover 120 and/or pillow 140 may provide a soft or atraumatic surface to prevent the implant frame 160 from damaging the tissue (e.g., the native leaflets of the heart valve).

FIG. 1B is a schematic diagram of an implant 200, according to an embodiment. As shown, the implant may include a first arm (e.g., an atrial arm 262) and a second arm (e.g., a ventricular arm 264) and a central portion 266 therebetween. The atrial arm 262 and at least a portion of the ventricular arm 264 may form the attachment section, and the central portion 266 and the support members 280 may form at least a portion of the coaptation section, as described in FIG. 1. In some embodiments, the implant frame may further comprise one or more friction elements (not shown) configured to apply a friction force to heart valve tissue to secure the implant 200 to the leaflet of the heart valve. The one or more friction elements may extend from at least one of the atrial arm 262 or the ventricular arm 264 preventing unintentional movement and/or removal of the implant during and/or after implantation.

In some embodiments, one or more sutures may be wrapped around one or more portions of the implant frame. In some embodiments, the implant frame may be entirely wrapped in one or more sutures. In some embodiments, a portion of the implant frame may be wrapped in one or more sutures. In some embodiments, the suture 210 may be secured (e.g., knotted) to a portion (e.g., a central portion 266) of the implant frame such that the suture has a first free end and a second free end. For example, the suture 210 may be secured to the central portion 266 at a base of the ventricular arm 264. In some embodiments, the first free end may be configured to be wrapped around the central portion 266 and the atrial arm 262. In some embodiments, the second end portion may be configured to couple the cover 220 to the implant frame 260. In some embodiments, the second free end may be configured to wrap around a portion of the implant (e.g., the ventricular arm 264). In some embodiments, the cover 220 may include a central cover 222 configured to be disposed around the central portion 266 and the support members 280. The central cover 222 may envelop the support members 280 to form the coaptation section of the implant. In some embodiments, the cover 220 may include a ventricular sock 224 configured to be disposed over the ventricular arm 264 and coupled to the central cover 222. In some embodiments, the central cover 222 and/or the ventricular sock 224 may be secured to the implant frame via the suture 210. In some embodiments, the central cover 222 may form or be coupled to a pillow 228. In some embodiments, the pillow 228 may extend from the distal end of the implant 200 and form a portion of the coaptation surface and/or to extend the coaptation surface.

In some embodiments, the ventricular sock 224 may be formed by aligning two pieces of fabric and cauterizing three of the edges. Alternatively, the ventricular sock 224 may be formed by folding a single piece of fabric in half and cauterizing two of the edges. In some embodiments, the edges of the fabric may be cauterized such that the ventricular sock 224 has a low profile. The term “profile” as described herein refers to a thickness the ventricular sock 224 extends from the implant frame. In some embodiments, a low profile refers to a profile low enough that the arms of the implant form an angle lower than about 15 degrees, lower than about 10 degrees, lower than about 5 degrees, lower than about 2 degrees when at rest. In some embodiments, a low profile refers to a profile lower than about 5 mm, lower than about 4 mm, lower than about 3 mm, lower than about 2 mm, lower than about 1, mm off of the implant frame. In some embodiments, the profile of the cover (i.e., the ventricular sock 224) may be low enough such that the arms of the implant sit coplanar (e.g., form an angle of about 0 degrees). In some embodiments, cauterizing the edges of the ventricular sock 224 can maintain a shape of the ventricular sock 224 and/or provide stability (i.e., a tensioned or taut surface) for suturing. Therefore, cauterizing the edges can enable an even seam, whereas suturing alone can cause clumping and/or puckering of the edges of the ventricular sock 224. In some embodiments, cauterizing the edges of the ventricular sock 224 may result in the ventricular sock 224 having a lower profile than if the edges of the ventricular sock 224 were stitched. In some embodiments, the low profile of the ventricular sock 224 may enable the ventricular arm 264 and the atrial arm 262 to sit near planar when the implant 200 is in the closed configuration. Additionally, cauterizing or fusing the edges can enable easier assembly and/or manufacturing by reducing an amount of suturing to form the cover. For example, covers configured to be disposed around the first and/or second arms of the implant, for example, (which have a small area) can be formed with fewer or no stitches, which can be difficult to perform. In some embodiments, a portion of the suture 210 may be configured to pass through (e.g., running stitch) the cauterized edges of the ventricular sock 224 to secure the ventricular sock 224 to the ventricular arm 264. In some embodiments, the second free end of the suture 210 may be configured to pass through the cauterized edges of the ventricular sock 224 to secure the ventricular sock 224 to the implant frame.

In some embodiments, the cover 220 may define one or more openings that align with one or more openings defined by the implant frame. In some embodiments, the cover 220 may be cauterized to form openings on the cover 220 that align with the openings of the implant frame. In some embodiments, the atrial arm 262 and/or the ventricular arm 264 may define one or more openings (e.g., also referred to herein as “tether holes”). The one or more openings of each arm 262, 264 may be configured to receive a respective tether such that the tether can actuate the atrial arm 262 and/or the ventricular arm 264 when tension is applied to the tether(s). In some embodiments, the cover 220 may define the one or more holes such that the tether(s) can be fed through at least one of the atrial arm 262 or the ventricular arm 264. Therefore, the atrial arm and/or the ventricular arm may be actuated (e.g., separated or opened) via the tethers to couple the implant 200 to the native leaflet of the heart valve. The implant 200 may be structurally and/or functionally similar to the implant 100, and therefore, certain details of the implant 200 are not described herein with respect to FIG. 1B.

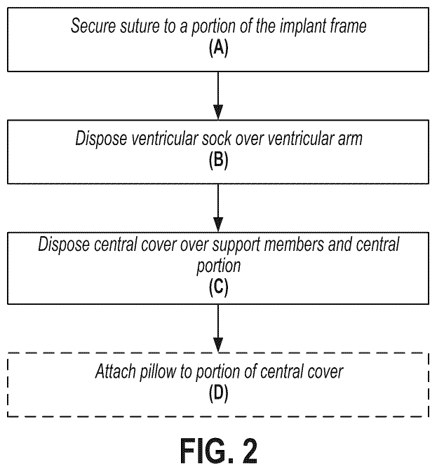

FIG. 2 is a flow chart of an example method for covering a cardiac implant with a cover, according to an embodiment. It should be appreciated that the steps of the method of FIG. 2 can be performed in any suitable way and can be performed in an order different than illustrated. All such variations are within the scope of this application. In some embodiments, the method may include securing a suture to a portion of the implant frame (e.g., an atrial arm of the implant), at A. In some embodiments, the suture may be secured to the atrial arm and/or the central portion (e.g., a spine) of the implant. The suture may be secured to the implant by forming loops or wraps around the implant with the suture. The loops or wraps may be formed tightly and close together along the implant such that the implant is not exposed and the loops or wraps do not slide relative to the implant. In some embodiments, the method may include disposing a ventricular sock (e.g., the second portion of the cover) over the ventricular arm of the implant, at B. In some embodiments, the ventricular sock can be coupled to the ventricular arm of the implant before the suture is secured to the implant. In some embodiments, the method may include disposing a central cover (e.g., the first portion of the cover) over support members and a central portion of the implant, at C. In some embodiments, the method may optionally include forming or attaching a pillow to a portion of the central cover (e.g., at a distal or bottom end of the central cover), at D. In some embodiments, the atrial arm may be wrapped with the suture before the ventricular sock is disposed over the ventricular arm. In some embodiments, the ventricular sock may be disposed over the ventricular arm before the atrial arm is wrapped with the suture. In some embodiments, the steps A and B may occur concurrently or partially concurrently.

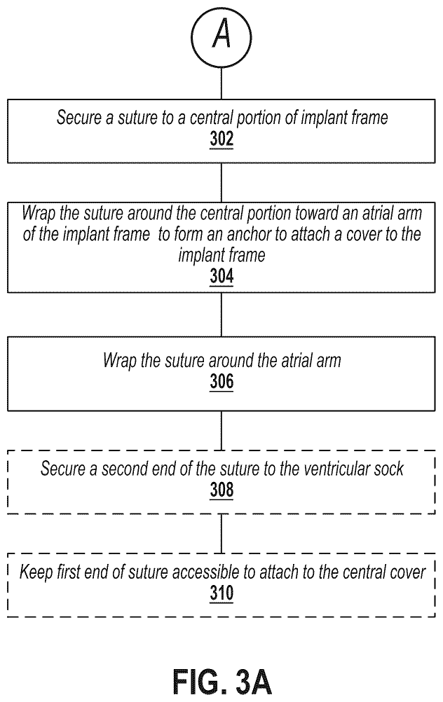

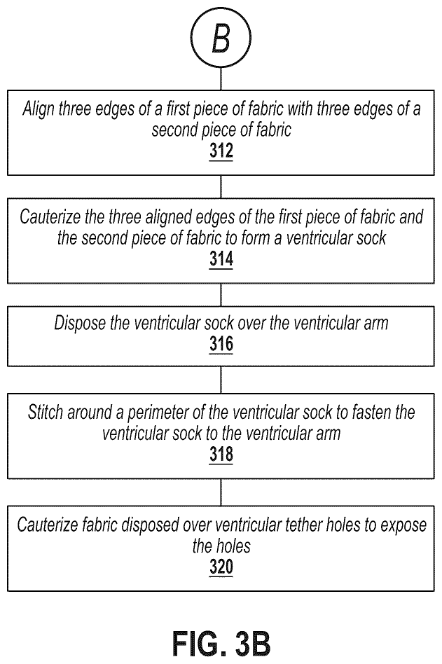

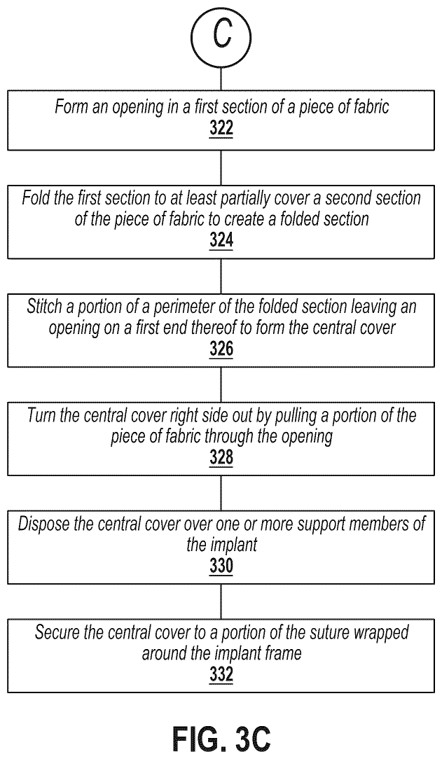

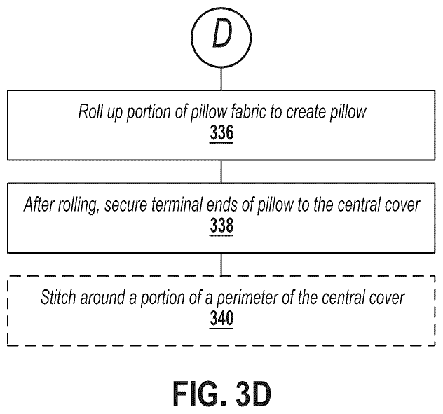

FIG. 3A is a flow chart of an example method A for securing the suture to the atrial arm of the implant. In some embodiments, the method A may include securing the suture to a central portion of an implant frame, at 302. For example, the suture may be tied to a portion of the central portion of the implant frame such that the suture has a first free end and a second free end. In some embodiments, the first free end may be longer than the second free end. In some embodiments, the suture may be coupled to the central portion near a transition from the central portion to the ventricular arm. In some embodiments, the suture may be wrapped around the central portion toward an atrial arm of the implant frame, at 304. In some embodiments, wrapping the suture around the central portion can form an anchor to attach a cover to the implant frame. In some embodiments, each loop around the central portion may contact the adjacent loops such that no gaps exist between the loops. In some embodiments, the method may include wrapping the suture around the atrial arm, at 306. In some embodiments, the suture may be wrapped around the atrial arm to cover the atrial arm (e.g., to cover one or more sharp edges of the atrial arm), thereby preventing damage of the tissue by the atrial arm. In some embodiments, the suture may be wrapped around the atrial arm such that one or more openings (e.g., tether holes) defined by the atrial arm are unobstructed. In some embodiments, the suture may be wrapped around the atrial arm such that one or more friction elements extending from the atrial arm are exposed or unobstructed. In some embodiments, a predetermined number of loops may be formed between each friction element. In some embodiments, a predetermined number of loops may be formed between each support member extending from the atrial arm of the implant. In some embodiments, the method may include securing the second end of the suture to a ventricular sock, at 308. In some embodiments, the method may include keeping the first end of the suture accessible to attach the central cover to the implant frame via the first end of the suture.

FIG. 3B is a flow chart of an example method B for forming a ventricular sock and disposing the ventricular sock over the ventricular arm of the implant. The method B may include aligning three edges of a first piece of fabric with three edges of a second piece of fabric, at 312. Alternatively, a first piece of fabric may folded in half, and two edges of the folded fabric may be aligned. The first piece of fabric and the second piece of fabric may have dimensions corresponding to dimensions of the ventricular arm. In some embodiments, the method B may include cauterizing the three aligned edges of the first piece of fabric and the second piece of fabric to form a ventricular sock, at 314. In some embodiments, cauterizing the edges of the ventricular sock may reduce a profile (e.g., a thickness off of the implant frame) of the ventricular sock, and therefore, prevent the ventricular sock from disrupting functionality of the implant (e.g., the clamping of the implant). Alternatively, the three aligned edges of the ventricular sock may be stitched instead of cauterized. In some embodiments, an end portion of each of the first piece of fabric and the second piece may be folded. In some embodiments, edges of the folded portion of the first piece of fabric and the second piece of fabric may be cauterized to form a pocket on each of the first piece of fabric and the second piece of fabric.

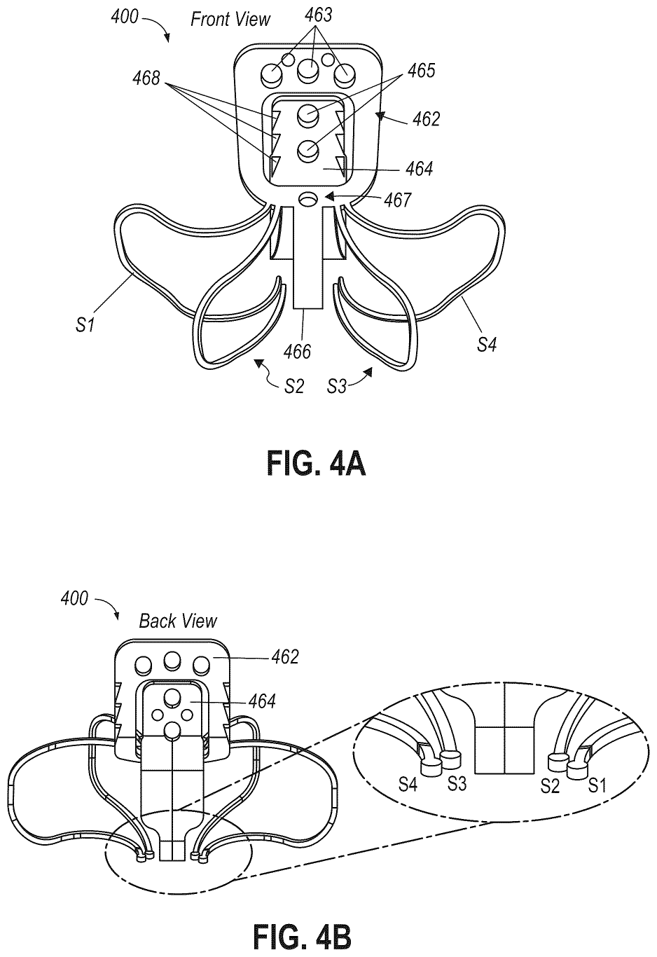

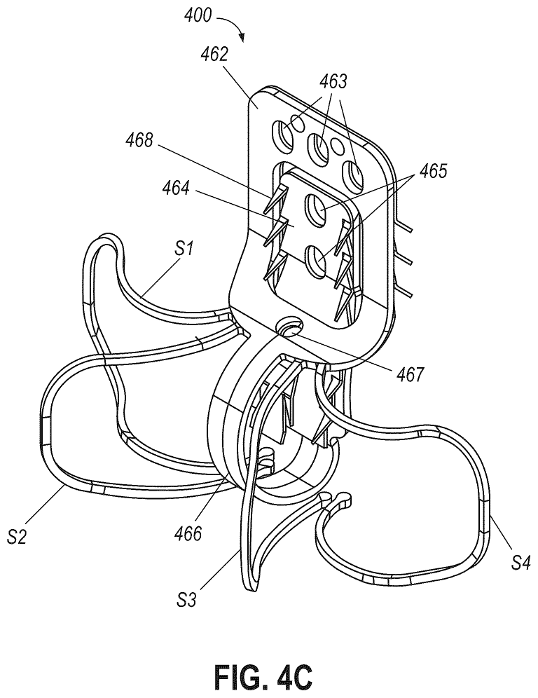

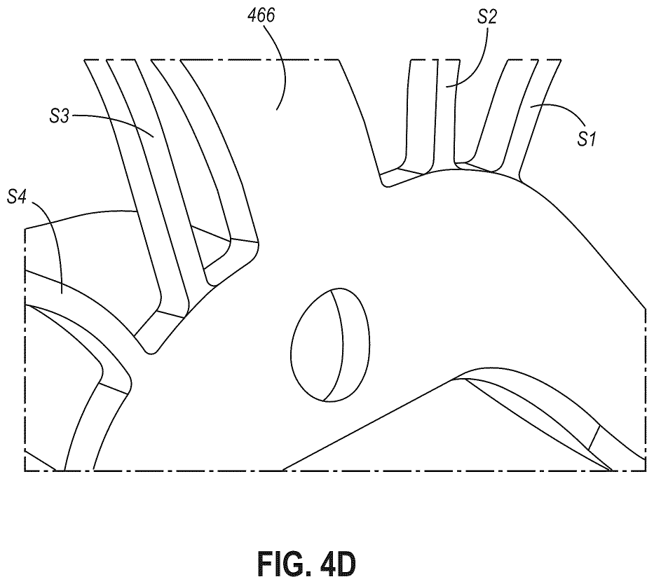

In some embodiments, the method B may include disposing the ventricular sock over the ventricular arm, at 316. For example, an opening (e.g., the edges of the ventricular sock that are not cauterized) may be disposed over the ventricular arm, and the ventricular sock may be pulled down the ventricular arm (e.g., distally, towards the central portion). Deflection of the support members during coupling of the cover to the implant can impart unwanted strain on the support members and weaken the integrity of the metal frame of the implant. For example, weaving the suture around the support members to secure the central cover can further causes strain and lead to weakening of the support members, particularly at transition points between the atrial arm and the support members (e.g., as shown in FIG. 4D). Therefore, the suture, ventricular sock, and/or central cover may be coupled to the implant in manner to prevent strain to be imparted on the support members and/or deflection of the support members.

In some embodiments, the ventricular arm may include one or more friction elements, and the method B may include piercing the ventricular sock with the friction elements once the ventricular sock is in place relative to the ventricular arm. Therefore, the friction elements can be exposed through the ventricular sock. The method B may include stitching around a perimeter of the ventricular sock to fasten the ventricular sock to the ventricular arm, at 318. In some embodiments, the method may include cauterizing fabric disposed over one or more openings (e.g., tether holes) defined by the ventricular arm to expose the one or more openings, at 320. Therefore, one or more tethers may be disposed through the openings defined by the ventricular arm to actuate the ventricular arm during implantation.

FIG. 3C is a flow chart of an example method C for forming a central cover and coupling the central cover to the implant frame. In some embodiments, the central cover may be formed from a piece of fabric having at least a first section and a second section. In some embodiments, the cover may be formed from a piece of fabric having a first section, a second section, and a third section. In some embodiments, the method may include forming an opening in the first section of the piece of fabric, at 322. For example, two flaps may be cut into the first section of the piece of fabric, and the flaps may be sewn open. In some embodiments, the method may include folding the first section to at least partially cover the second section of the piece of fabric to create a folded section at 324. In some embodiments, the method may include stitching a portion of a perimeter of the folded section leaving an opening on a first end thereof to form the central cover, at 326. In some embodiments, the method C may include turning the central cover right side out by pulling a portion of fabric through the opening (i.e., the cover is pulled such that a surface of the cover configured to face outward is pulled to face outward), at 328. Therefore, the stitching around the perimeter may be disposed on an inside of the central cover. In some embodiments, the method C may include disposing the central cover over one or more support members of the implant, at 330. For example, the one or more support members may be disposed through an opening of the central cover such that the central cover envelopes the support members. In some embodiments, the method may include securing the central cover to a portion of the suture wrapped around the implant frame (e.g., the atrial arm and/or the central portion), at 332.

FIG. 3D is a flow chart of an example method D for forming a pillow coupleable to the implant. In some embodiments, the method D may include rolling up a portion of pillow fabric to create the pillow, at 336. In some embodiments, the pillow fabric may be continuous with the central cover fabric (e.g., the third section of the central cover fabric). In some embodiments, the method D may include, securing, after rolling the pillow fabric, the terminal ends of the pillow to the central cover, at 338. In some embodiments, the terminal ends of the pillow may be temporarily secured to the central cover while edges of the pillow are stitched to the central cover. In some embodiments, the method D may include stitching around a portion of a perimeter of the pillow and/or central cover to attach the pillow to the central cover, at 340. In some embodiments, after the portion of the perimeter is stitched, the temporary stitching of the terminal ends of the pillow may be removed.

In some embodiments, the pillow may have different shapes or stiffnesses (e.g., corresponding to the anatomy). In some embodiments, the pillow may have any suitably shaped cross-sectional area such as, for example, a circular, oval, or an irregular cross-sectional area. In some embodiments, the pillow may be formed to have a stiffness in a predetermined range. For example, the pillow may have a stiffness greater than a lower threshold such that the pillow does not deform in response to blood flow. The pillow may have a stiffness lower than a higher threshold such that the pillow can deform in response to contact from the leaflet to prevent damage to the leaflet.

FIG. 4A is a perspective front view of a cardiac implant 400 (hereinafter, “implant”) and FIG. 4B is a perspective back view of the cardiac implant 400, according to embodiments. As shown, the implant 400 includes a first arm 462 and a second arm 464 coupled to one another by a central portion or spine 466. The first arm 462 (e.g., the atrial arm) may be configured to contact a side of the native leaflet of a heart valve that faces an atrium of the patient and the second arm 464 (e.g., the ventricular arm) may be configured to contact a second side of the native leaflet of the heart valve that faces a ventricle of the patient. In some embodiments, the first arm 462 may include one or more openings 463 configured to receive a first tether. The second arm 464 may include one or more openings 465 configured to receive a second tether. In some embodiments, the first tether and the second tether may be configured to extend through a delivery system for the implant and be coupled to a handle assembly of the delivery system. In some embodiments, a base of the atrial arm may include an opening 467. In some embodiments, the opening 467 may be configured to receive a tether. Each of the first arm 462 and the second arm 464 may include one or more friction elements 468 configured to secure each of the first arm 462 and the second arm 464 to the leaflet of the heart valve. In some embodiments, the implant 400 may include four support members S1, S2, S3, S4 extending from a base of the first arm 462 to a free end. In some embodiments, a cover may be configured to be coupled to the implant 400 to cover at least a portion of the implant 400. In some embodiments, the portion of the cover may be disposed around the support members S1, S2, S3, S4 to create a coaptation surface.

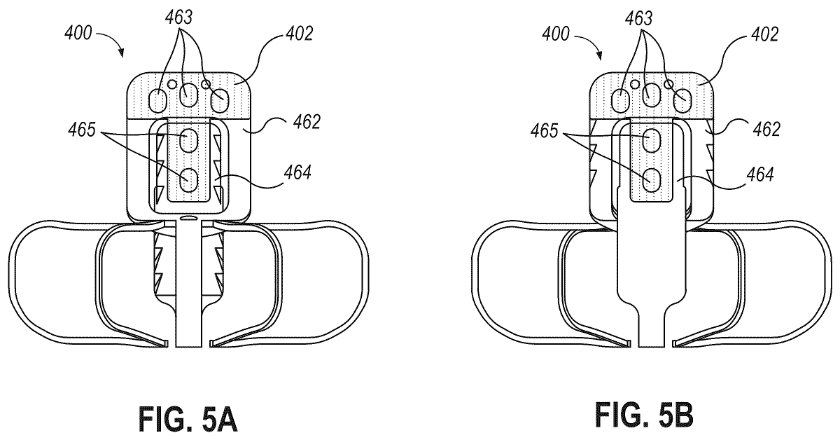

FIGS. 5A-5B show a portion 402 of the implant 400 for a user to handle while applying a cover to the implant 400, according to embodiments. As shown, a top portion of the first arm (e.g., near and around the tether holes of the first arm) and a top portion of the second arm (e.g., near and around the tether holes of the second arm) may be engaged by the user. In some embodiments, the user may hold the predetermined portions of the implant 400 to prevent damage (e.g., bending deformation) of the implant 400. For example, the support members may not be engaged by the user to prevent damage of the coaptation portion of the implant.

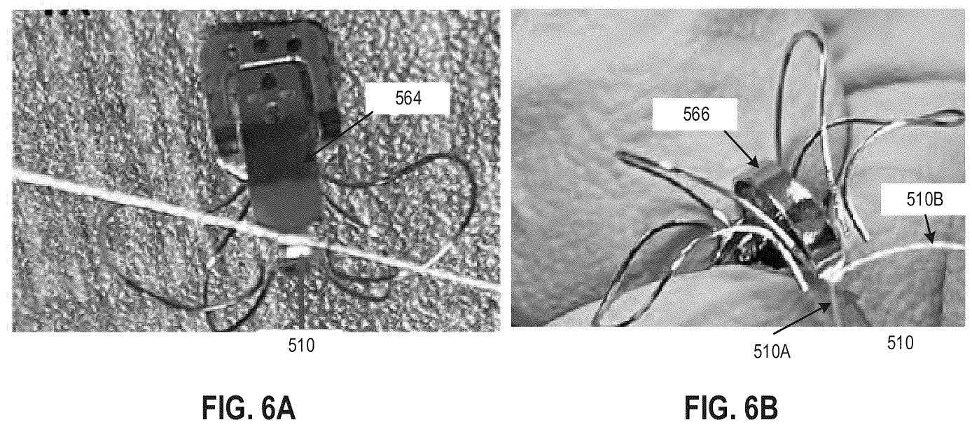

Embodiments described herein may include a method for covering the implant frame. FIGS. 6A-52 outline steps of an example method of covering the implant frame. In some embodiments, any one of or all of the following steps may be performed inside a controlled environment room.

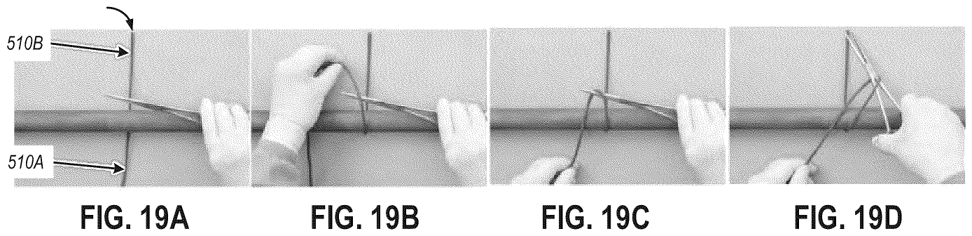

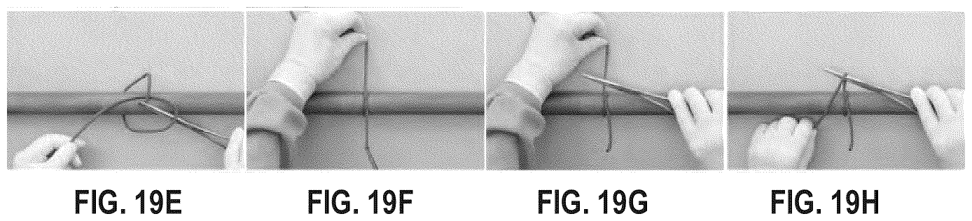

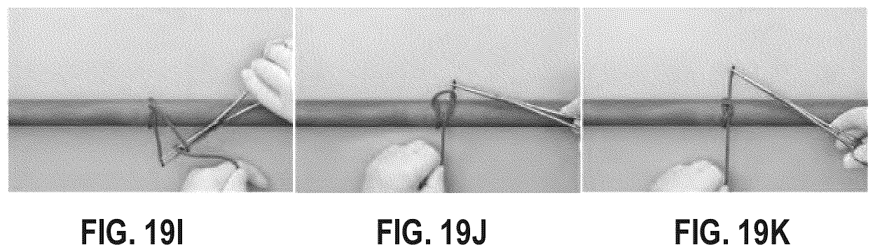

To couple a cover to the implant, a portion of the implant is wrapped in a suture. In some embodiments, the implant may be at least partially wrapped with a suture to provide an anchor for coupling one or more covers or portions of a cover to the implant frame (hereinafter referred to as “the implant”). As shown in FIGS. 6A-6B, the suture 510 may be knotted to the spine 566 of the implant leaving a first free end (hereinafter, “the first end”) 510A and a second free end (hereinafter, “the second end”) 510B. In some embodiments, the first end 510A may be longer than the second end 510B. In some embodiments, the second end 510B may be about 10 cm to about 20 cm, inclusive of all ranges and subranges therebetween. In some embodiments, the first end 510A may be about 10 cm to about 70 cm, inclusive of all ranges and subranges therebetween. In some embodiments, the first end 510A may be about 10 cm to about 50 cm, inclusive of all ranges and subranges therebetween. In some embodiments, the suture 510 may be secured to the spine 566 using more than one type of knot. In some embodiments, the suture 510 may be secured to the spine 566 using a square knot and a left-right-left knot (i.e., 3 alternating knots). FIGS. 19A-19K show steps of forming the knot to secure the suture 510 to the spine of the implant. The first end 510A of the suture 510 can be wrapped around the spine, as shown in FIGS. 19A-19B. A portion of the first end 510A can be folded over forceps to form a loop, shown in FIGS. 19C-19D. The second end 510B of the suture can be grabbed using the forceps and pulled through the loop formed by the first end portion 510A of the suture and pulled tight.

### Wrapping the Implant Frame

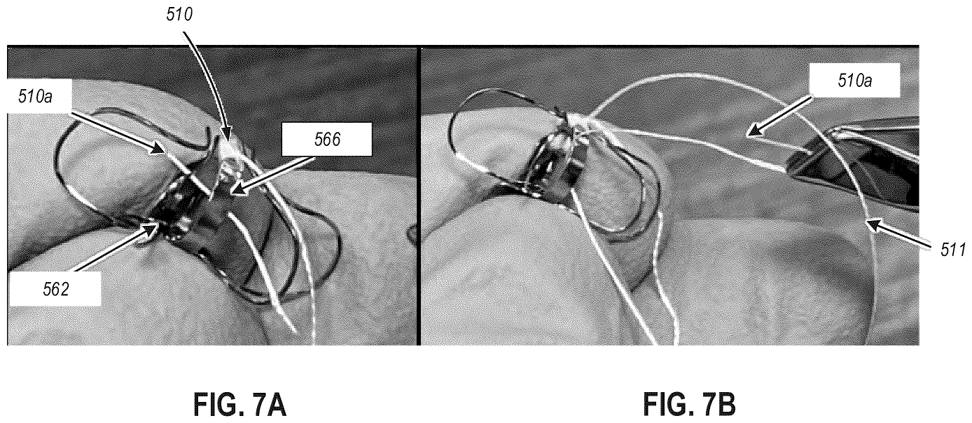

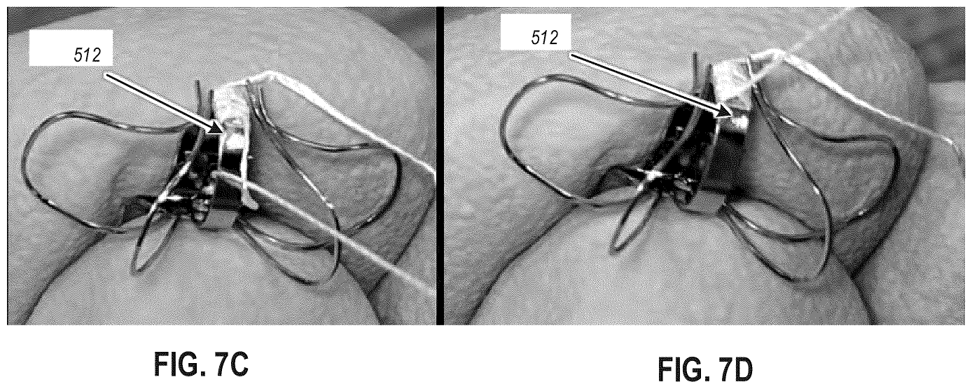

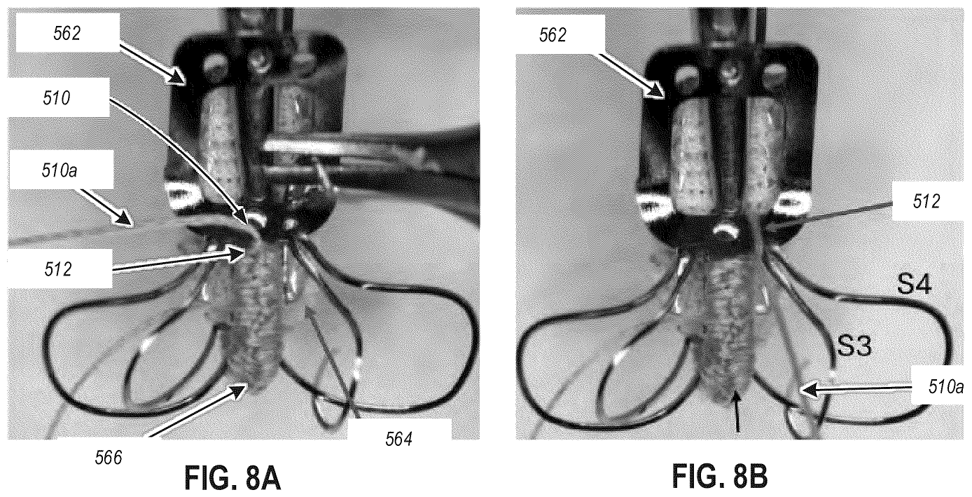

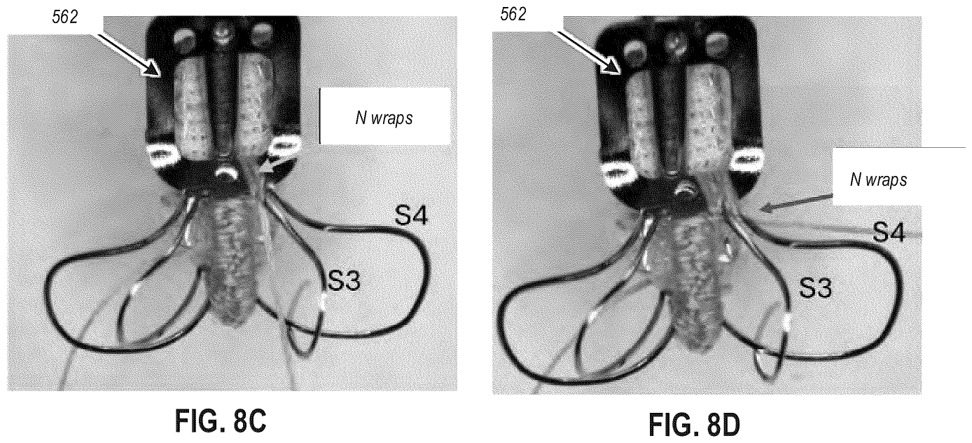

Referring back to FIGS. 6A-6B, the suture 510 can be secured to the spine 566 near a base of the ventricular arm 564. After the suture 510 is fixed to the spine 566, the method may include wrapping the suture 510 around the spine 566 towards the atrial arm 562. The wrapping of the suture 510 may include disposing the first end 510A of the suture 510 under the spine 566 (e.g., when the implant is oriented with the ventricular side up), as shown in FIG. 7A. The method can include pulling the first end 510A of the suture back over a top of the spine to form a loop 512, and threading the first end 510A of the suture through the loop 512 (e.g., in a clockwise direction), as shown in FIG. 7B. The method may include tightening the loop 512 so that the suture lies snug against the spine, as shown in FIG. 7C. The method may include repeating the steps of (i) disposing the suture 510 under the spine; (ii) pulling the first end 510A of the suture back over the top of the spine; (iii) threading the first end 510A of the suture through a loop 512; and (iv) tightening the loop 512 such that the suture wraps around the implant spine. In some embodiments, after each loop is tightened to form a portion of the suture wrap, the method may include sliding a respective loop to contact the previously formed loop to reduce gaps between adjacent suture loops, as shown in FIG. 7D. In some embodiments, the suture 510 may be wrapped around the spine 566 until the spine 566 is at least partially covered. In some embodiments, the suture 510 may be wrapped around the spine 566 until the spine 566 is fully covered. In some embodiments, the suture 510 may be wrapped around the spine 566 until a suture loop 512 sits at a base of the atrial arm 562, as shown in FIG. 8A. As shown, the first end 510A of the suture 510 is free for subsequent wrapping of the implant frame.

In some embodiments, once the spine 566 is fully wrapped, the method may include wrapping the atrial arm 562 from right to left (e.g., counterclockwise). For example, the first end 510A of the suture 510 may be disposed over a portion of the atrial arm 562 (e.g., from a front side of the implant toward a back side of the implant) to form a loop. The first end 510A of the suture 510 may then be pulled through the loop from the back side of the implant toward the front side of the implant, as shown in FIG. 8B. The wrapping may be repeated until there are a predetermined number N suture loops (e.g., 1-4 loops or wraps) around the atrial arm between the spine 566 and the third support member S3, as shown in FIG. 8C. The method may include disposing the first end of the suture underneath the third support member S3 and between the third support member S3 and the fourth support member S4 to form a loop. The method may include disposing the first end of the suture through the loop. The method may include repeating the wrapping until two loops are formed around the atrial arm 562 between the third support member S3 and the fourth support member S4. The method may include disposing the first end of the suture under the fourth support member S4 and continue wrapping along the atrial arm 562.

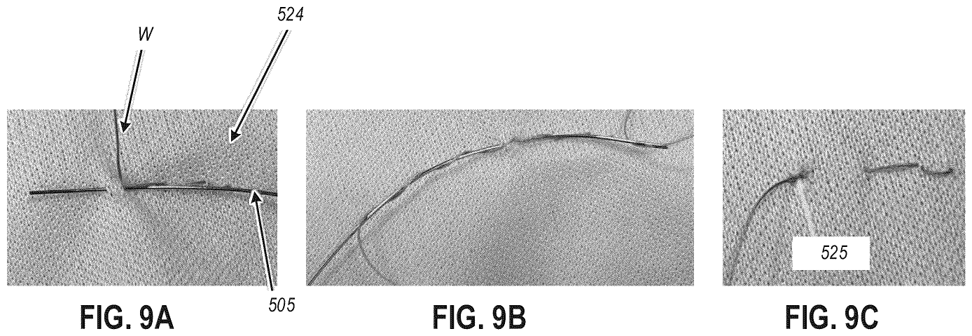

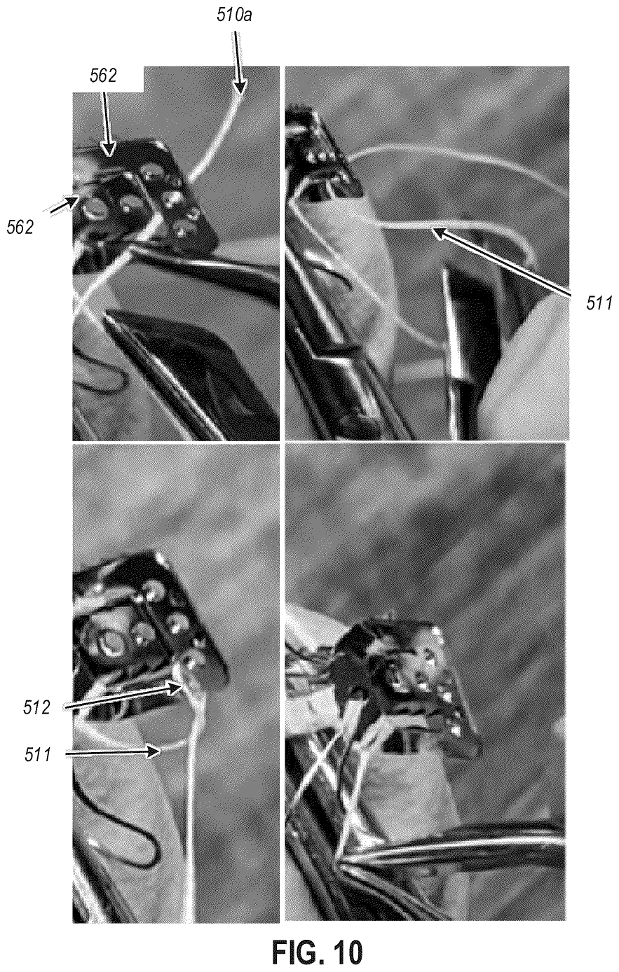

In some embodiments, the method may include tying off the second end of the suture used to secure the ventricular sock 524. In some embodiments, the wrapping of the atrial arm with the first end of the suture may be paused while the second end of the suture is used to secure the ventricular sock 524. In some embodiments, tying off the second end may occur before the atrial arm is fully wrapped. Tying off the second end may include passing the second end of the suture through one of the wraps on the spine and wrapping the non-leading suture end (i.e., denoted with W) around the end of the needle 505 a predetermined number of times (e.g., 1 time, 2 times, 3 times, 4 times, 5 times, etc.), as shown in FIGS. 9A-9B. In some embodiments, the method includes wrapping the end of the needle 3 times. The wrapped needle can be pulled through a portion of the ventricular sock to form a cinch knot 525, as shown in FIG. 9C. In some embodiments, the method may include trimming off the excess suture and/or cauterizing the knot to remove frayed ends. In some embodiments, an ionizing air gun can be used to remove particulars from a portion of the implant (e.g., the atrial arm and/or the spine) after cauterization. In embodiments in which the atrial arm wrapping is paused to tie off the second end of the suture, the wrapping of the atrial arm may continue after the second end of the suture is cauterized. FIG. 10 shows wrapping of a side portion of the atrial arm 562. In some embodiments, the first end 510A of the suture may be wrapped by disposing the suture through a gap between the atrial arm 562 and the ventricular arm 564. In some embodiments, the method may include forming a loop 511 and disposing the first end of 510A of the suture through the loop 511 to form a wrap around the atrial arm. The suture may be pulled tight such that the wrap is secured to the implant frame.

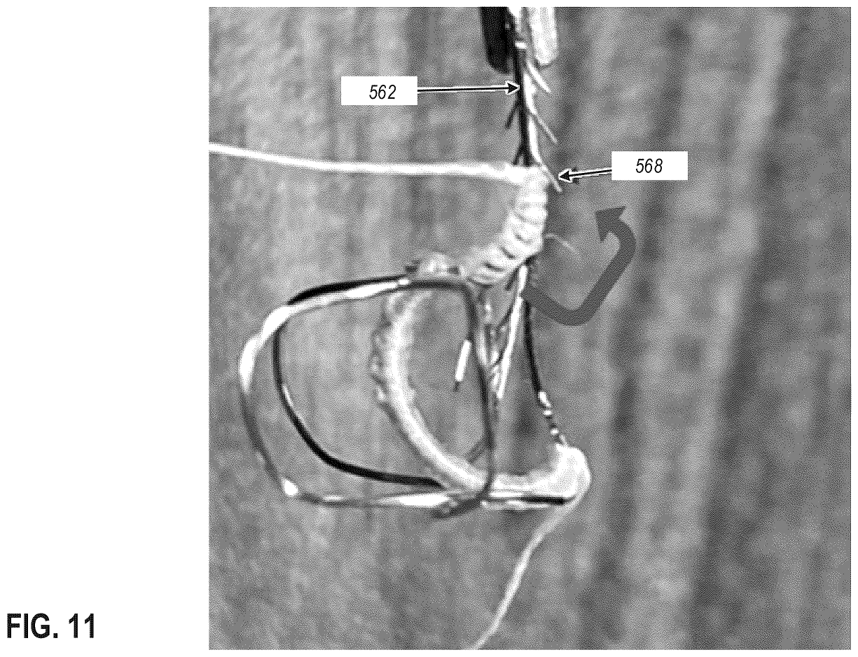

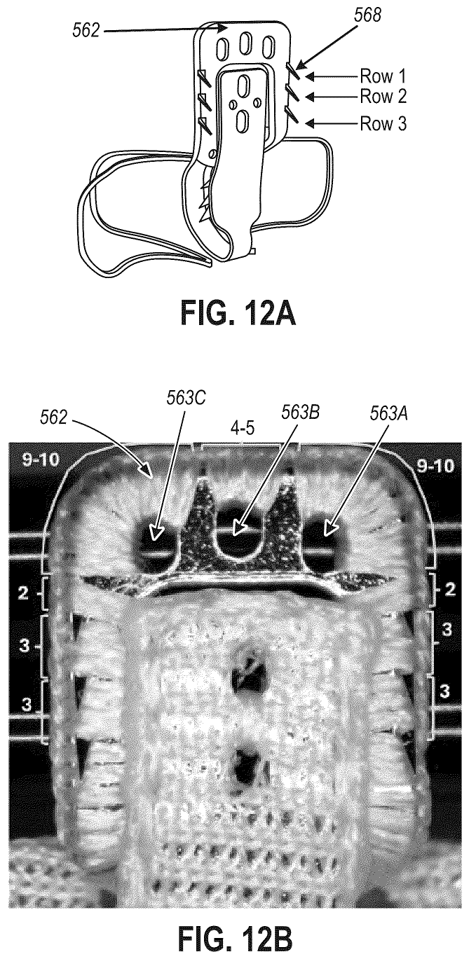

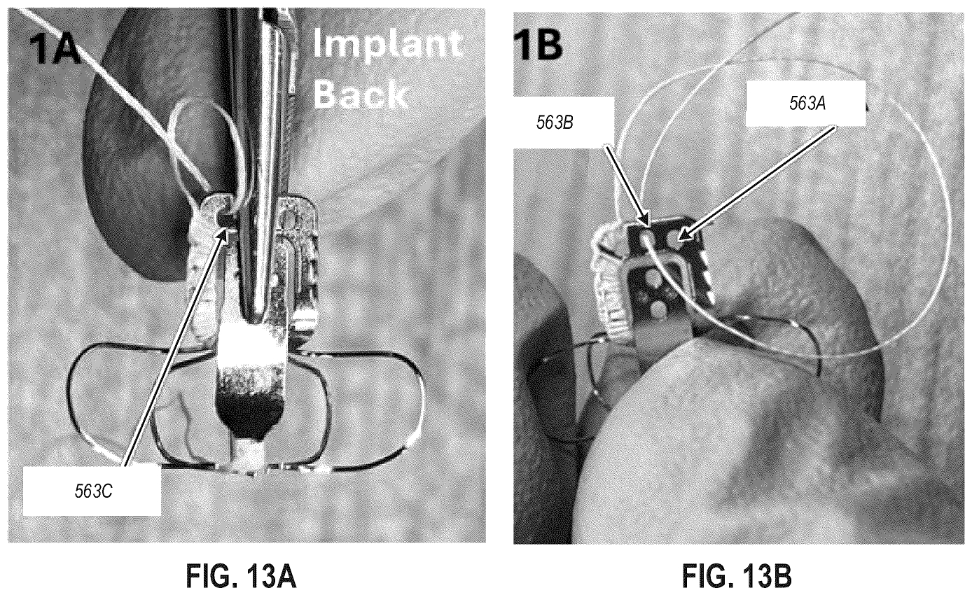

In some embodiments, the method may include aligning the loops of the suture along an outside edge of the atrial arm 562, as shown in FIGS. 11-12B. The atrial arm of the implant may have a plurality of friction elements 568. For example, the atrial arm 562 may have a first row R1, a second row R2, and a third row R3 of friction elements 568, as shown in FIG. 12A. As shown in FIG. 12B, a predetermined number of suture wraps may lie between each of the rows of friction elements. For example, three suture wraps may lie between the first row and the second row, three suture loops may lie between the second row and the third row, and two sutures between the third row and a bottom edge of the tether holes 563A, 563B, 563C. The method may include wrapping the atrial arm 562 until reaching the first tether hole 563A, and wrapping the suture a predetermined number of times until reaching the second tether hole 563B. For example, the predetermined number of times may be between about 8 to about 12 loops or wraps wrapped around each corner of the atrial arm 562. For example, 9-10 loops or wraps may wrap through the first tether hole 563A and 9-10 loops or wraps may wrap through the third tether hole 563C. In some embodiments, between about 4-5 suture loops or wraps may wrap through the second tether hole 563B. An image of the wrapping of the tether holes 563A, 563B, 563C of the atrial arm is shown in FIGS. 13A-13B.

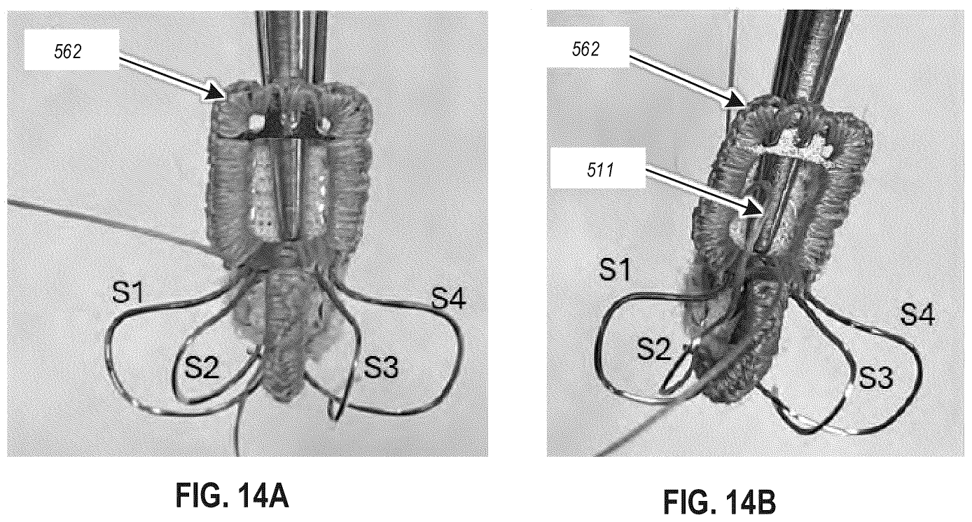

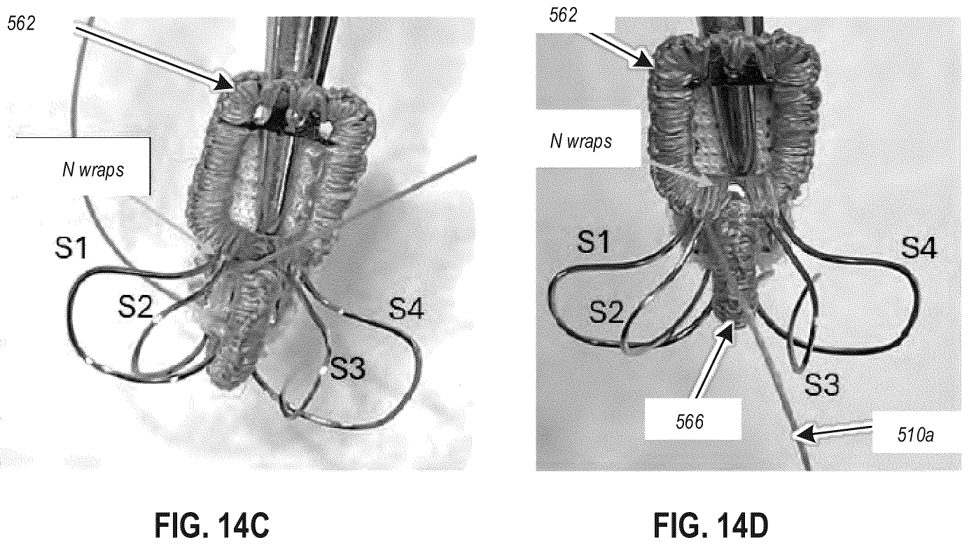

The method may include continuing to wrap the atrial arm 562 until the suture is close to the first and second support members S1, S2, as shown in FIG. 14A. Once the suture is near (e.g., adjacent to) the first support member S1, the method may include disposing the first end of the suture underneath S1 to form a loop 511, as shown in FIG. 14B. The method may include disposing the first end of the suture through the loop. The method may include wrapping a predetermined number N of loops or wraps (e.g., 1-4 wraps) between the first support member S1 and the second support member S2, FIG. 14C. The method may include wrapping the predetermined number N of wraps (e.g., 1-4 wraps) between the second support member S2 and the spine 566 of the implant, as shown in FIG. 14D. For example, two wraps or loops may be disposed between the first support member S1 and the second support member S2 and between the second support member S2 and the spine 566.

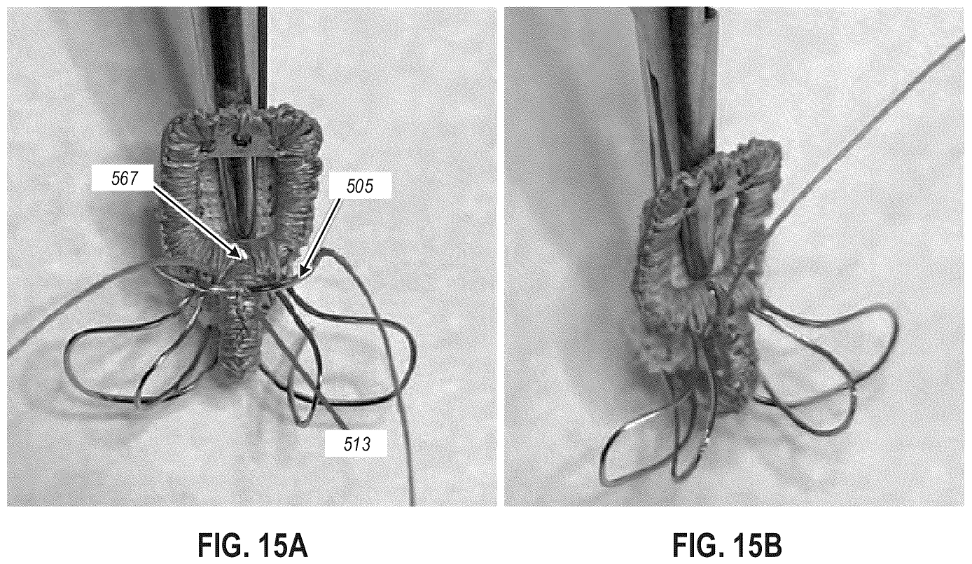

As shown in FIGS. 15A-15B, the suture needle 505 may be disposed through the suture loop or wrap 513 at a top of the spine. In some embodiments, the suture is not passed through the tether hole 567 at the base of the atrial arm (i.e., the fourth tether hole). In some embodiments, the first end of the suture may not be trimmed. For example, the excess tail of the first end of the suture may be used to attach the central cover. In some embodiments, the entire wrapping of the spine and the atrial arm may be performed using a single suture.

In some embodiments, wrapping the atrial arm may include verifying all the suture loops or wraps are lying flat in a center (e.g., a center surface) of the spine. In some embodiments, wrapping the atrial arm may include verifying all the suture loops are lying flat on an outside edge of the atrial arm. In some embodiments, wrapping the atrial arm may include verifying there are the predetermined number of loops between the spine and the support members. In some embodiments, wrapping the atrial arm may include verifying there are the predetermined number of loops between adjacent support members. In some embodiments, wrapping the atrial arm may include verifying the tether hole is open (e.g., no suture has been looped through any portion of the fourth tether hole). In some embodiments, wrapping the atrial arm may include verifying there are the predetermined number of suture loops between each row of friction elements. In some embodiments, wrapping the atrial arm may include verifying the atrial arm and the ventricular arm are free from connecting suture strands. In some embodiments, wrapping the atrial arm may include verifying the entire wrapping is performed using a single suture. In some embodiments, wrapping the atrial arm may include verifying no portion of the suture is frayed.

### Ventricular Sock

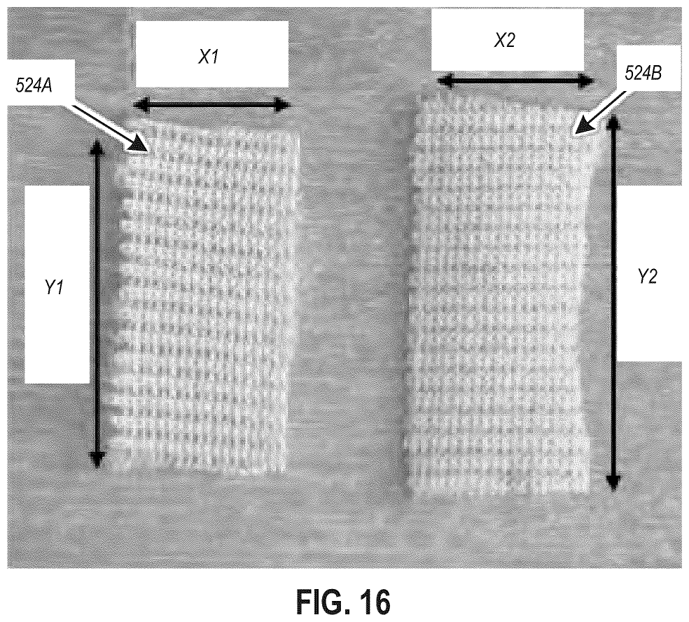

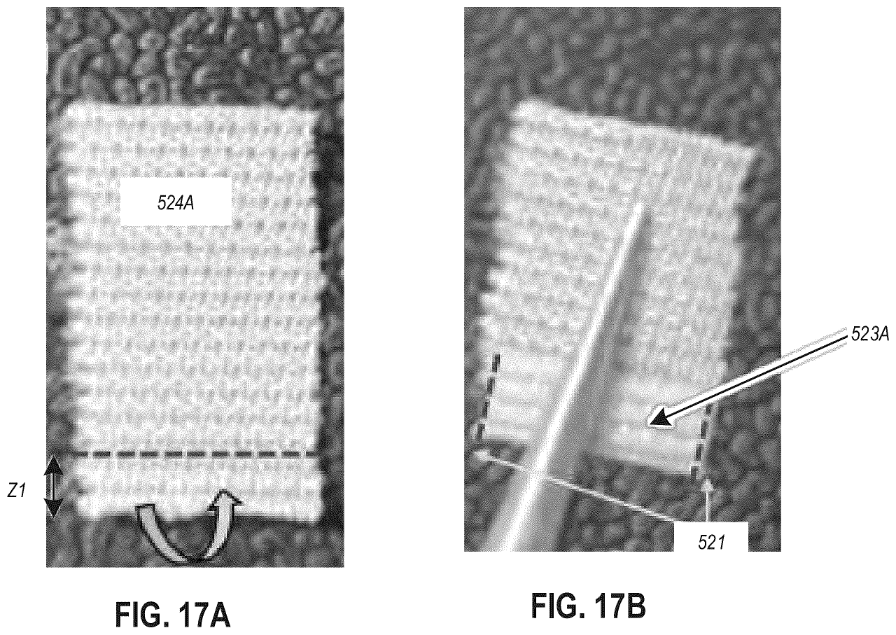

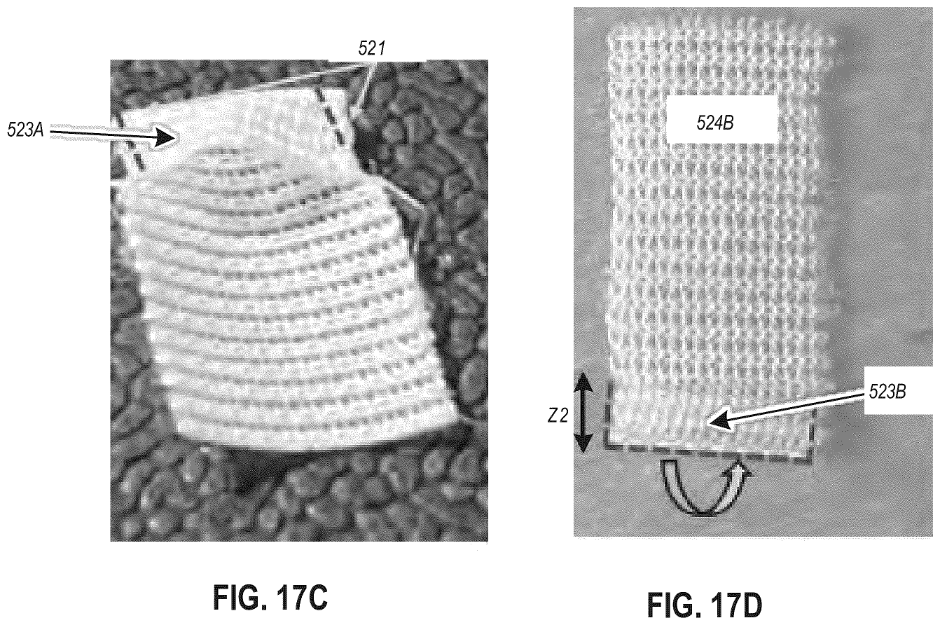

In some embodiments, the ventricular arm of the implant may be covered with a ventricular sock. In some embodiments, the ventricular sock may be formed from two pieces of fabric coupled to one another. In some embodiments, the pieces of fabric may have dimensions corresponding to the ventricular arm. As shown in FIG. 16, the first piece of fabric 524A may have a first width X1 and the second piece of fabric 524B may have a second width X2. In some embodiments, the first width X1 and the second width X2 may be equivalent. In some embodiments, the first piece of fabric 524A may have a first length Y1 and the second piece of fabric 524B may have a second length Y2. In some embodiments, the first length Y1 may be shorter than the second length Y2. The method of forming the ventricular sock may include folding a portion of the first piece of fabric 524A to create a first folded portion 523A. In some embodiments, a short side of the first piece of fabric 524A may be folded, as shown in FIGS. 17A-17B. In some embodiments, the short side may be folded a length Z1. In some embodiments, Z1 may be between about 0.5 mm to about 3 mm, inclusive of all ranges and subranges therebetween. In some embodiments, the method may include using calipers to measure the folded portion 523A. As shown in FIGS. 17B-17C, the short edges 521 of the folded portion 523A may be cauterized to form a first pocket. The method may include folding a short side of the second piece of fabric 524B, as shown in FIG. 17D. In some embodiments, the short side of the second piece of fabric 524B may be folded a second length Z2. In some embodiments, Z2 may be between about 0.5 mm and about 3 mm, inclusive of all ranges and subranges therebetween. In some embodiments, Z2 may be shorter than Z1. In some embodiments, short sides of the folded portion 523B of the second piece of fabric 524B may be cauterized to form a second pocket, as shown in FIG. 17D.

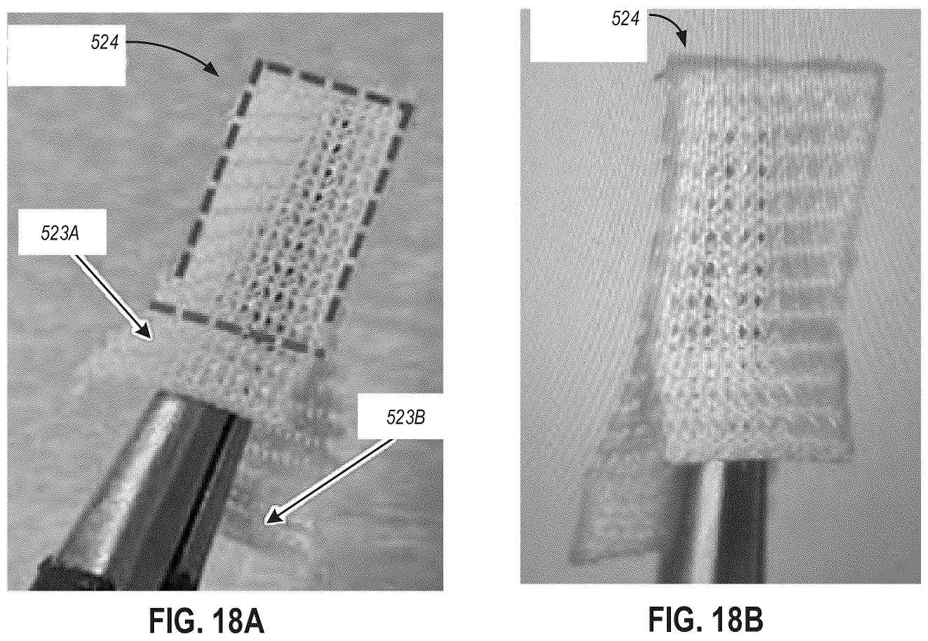

The method of forming the ventricular sock 524 may include aligning the first piece of fabric and the second piece of fabric such that the first pocket 523A and the second pocket 523B are aligned with one another and are facing one another, as shown in FIG. 18A. The first piece of fabric and the second piece of fabric may be positioned such that non-folded ends opposite the folded portions of the first piece of fabric and the second piece of fabric are substantially aligned. The method may include cauterizing around a periphery of the first piece of fabric and the second piece of fabric (e.g. shown with blue dotted line) to couple the first piece of fabric to the second piece of fabric and to form the ventricular sock 524. In some embodiments, a portion of fabric below the red dotted line may not be cauterized. In some embodiments, the top corners (e.g., corners opposite an open end of the ventricular sock 524) may be cauterized to at least partially round the corners, as shown in FIG. 18B.

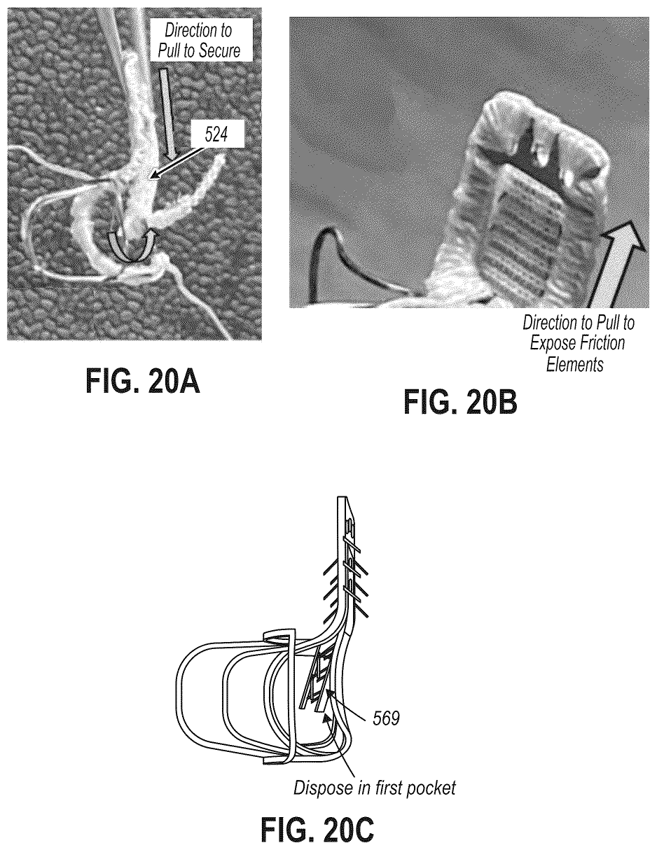

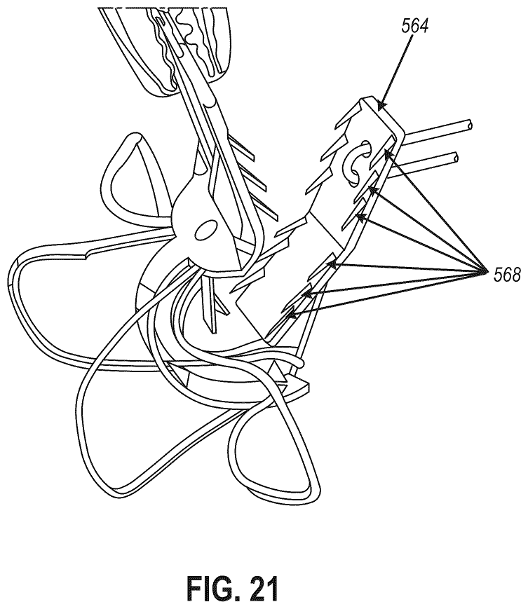

Once the ventricular sock is formed, the ventricular sock may be disposed over the ventricular arm of the implant. The method may include holding the implant using soft tissue or cloth and/or tweezers or graspers (e.g., rubber tipped) to avoid damaging the metal frame of the implant (see FIG. 5). The method may include pulling the ventricular sock 524 down onto the ventricular arm, as shown in FIG. 20A. In some embodiments, the ventricular sock may be disposed over the ventricular arm such that the first piece of fabric (e.g., the shorter length fabric) may be positioned on a front side of the implant and the second piece of fabric (e.g., the longer length fabric) may be positioned on a back side of the implant (see FIG. 4 to show front and back side of implant). In some embodiments the ventricular sock may be pulled down the ventricular arm, and the folded portion (e.g., the pocket) of the first piece of fabric is disposed over a friction element plate 569 of the ventricular arm, as shown in FIG. 20C. The folded portion may be disposed over the friction element plate 569 such that the folded portion covers the friction element plate 569. The method may include adjusting tension to the ventricular sock so that the friction elements (e.g., all of the friction elements) protrude out of the ventricular sock. In some embodiments, after the ventricular sock 524 is pulled down (e.g., toward the central portion), the ventricular sock 524 may be pulled upward (shown in FIG. 20B) to allow the friction elements to penetrate through the ventricular sock 524. In some embodiments, the ventricular arm 564 may have a total of 12 friction elements 568 (e.g., 6 friction elements on each side), as shown in FIG. 21.

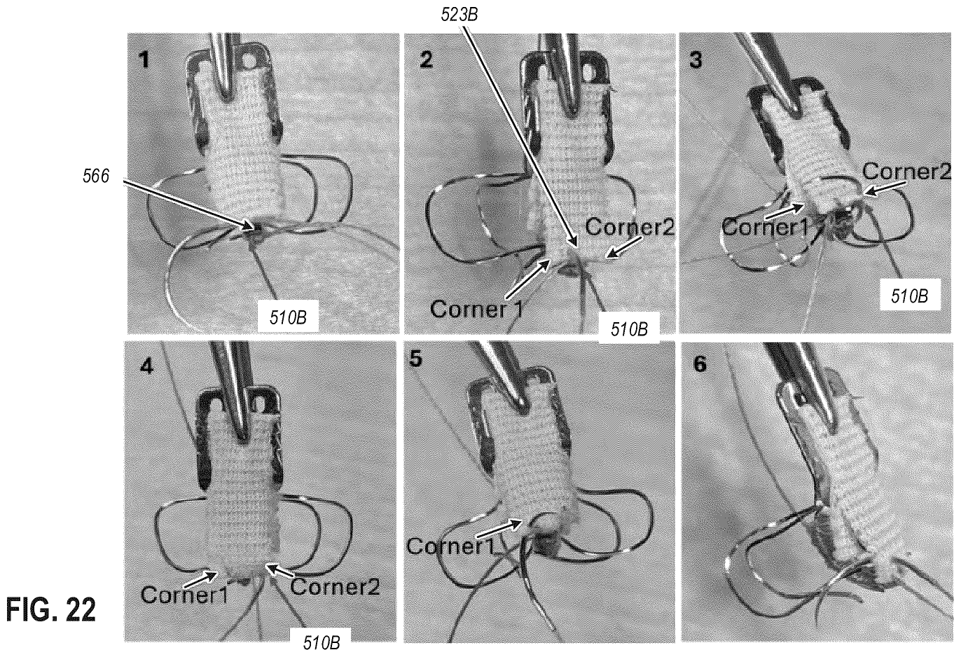

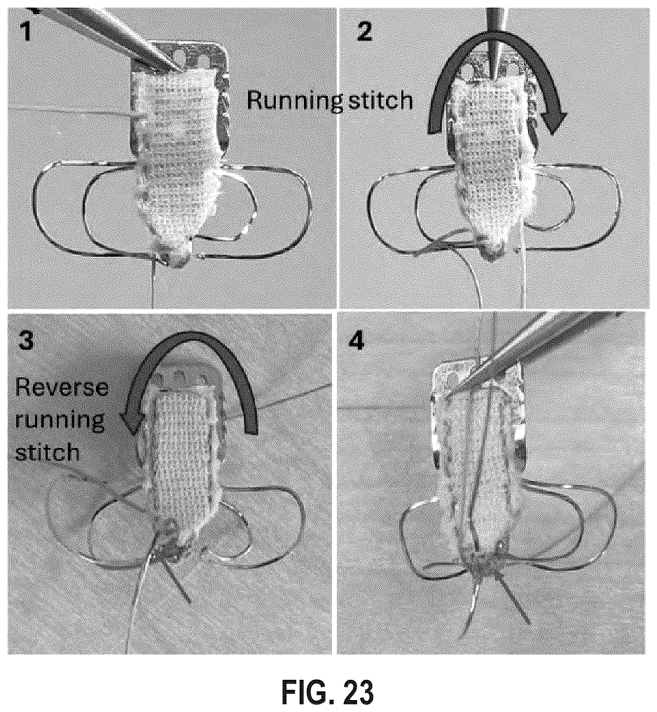

Once the ventricular sock is disposed over the ventricular arm, the ventricular sock may be secured to the implant frame. In some embodiments, to secure the ventricular sock to the frame, a portion of the ventricular sock may be secured to one or more suture loops on the implant spine, as shown in FIG. 22. For example, a portion of the ventricular sock that extends beyond the ventricular arm (e.g., the folded portion 523B of the second piece of fabric of the ventricular sock) may be stitched to a portion of the suture wrapped around the implant spine (see FIG. 22, panel 2 and panel 3). In some embodiments, the second end of the suture 510B may be passed through (e.g., using a needle) a center edge of the ventricular spine 566, as shown by the arrows in FIG. 22. In some embodiments, the needle threaded with the second end of the suture 510B may be passed near an edge of a first corner (labeled “Corner1”) of the ventricular sock, passed underneath the spine 566, and between a second corner (labeled “Corner2”) of the spine (FIG. 22, panel 2). In some embodiments, the needle threaded with the second end of the suture 510B may be passed to the edge of the second corner, and then passed underneath the spine 566 (shown in FIG. 22, panels 3-4). In some embodiments, the needle threaded with the second end of the suture 510B may be moved out at the edge of the first corner (shown in FIG. 22, panel 5). Once a bottom edge of the ventricular sock has been coupled to the spine, the method may include performing a running stitch along the edges of the ventricular sock, as shown in FIG. 23. In some embodiments, the running stitch may be performed clockwise. In some embodiments, the running stitch may be performed counterclockwise (e.g., a reverse running stitch). In some embodiments, the running stitch may be performed and then the reverse running stitch may be performed back to the starting point.

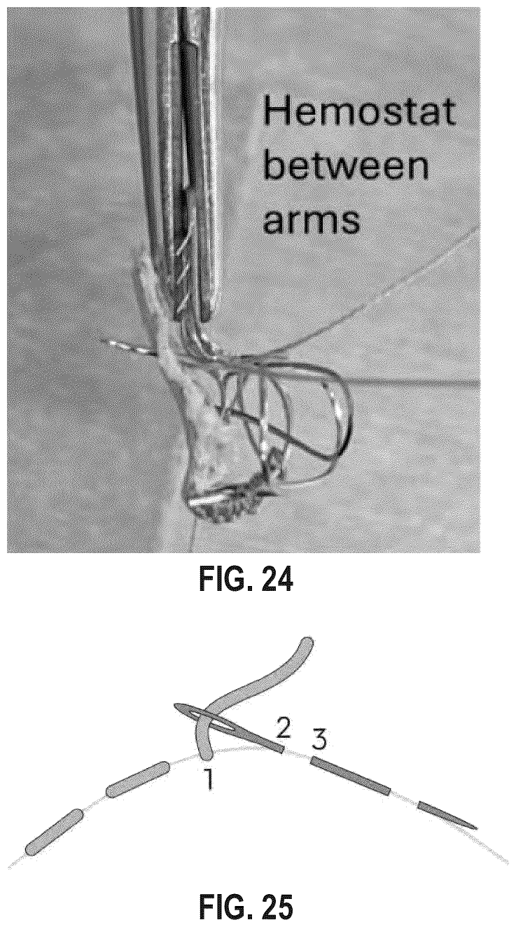

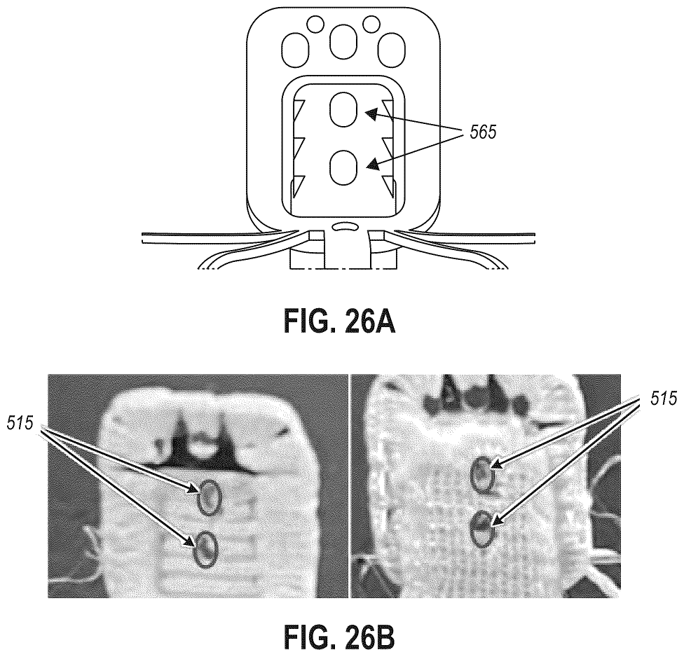

In some embodiments, the atrial arm and the ventricular arm of the implant may be held open (e.g., using a spacer such as a hemostat, as shown in FIG. 24) while performing the running stitch. FIG. 25 shows a pattern of moving the needle to achieve the running stitch. In some embodiments, the running stitch may be performed along the edges of the ventricular arm such that the stitches lie along a perimeter or edge of the implant frame and do not interfere with the friction elements. In some embodiments, the method may include performing a predetermined number of stitches along each side of the ventricular arm. For example, each long side of the ventricular arm may include 5 to 6 stitches. In some embodiments, each stitch should be less than about 2 rows on the knit fabric (e.g., between about 0.5 mm and about 1.5 mm long, inclusive of all ranges and subranges therebetween). The stitching of the ventricular arm may cause the sock to fit snug around the ventricular arm such that the sock stays in place (e.g., does not slip). In some embodiments, the stitching may go all the way to the tail of the spine and then the needle may be passed into the fabric. In some embodiments, once the ventricular arm is stitched to the suture around the spine, the second end of the suture may be left unfastened. In some embodiments, the stitching of the ventricular arm sock may further reduce a profile of the ventricular arm sock.

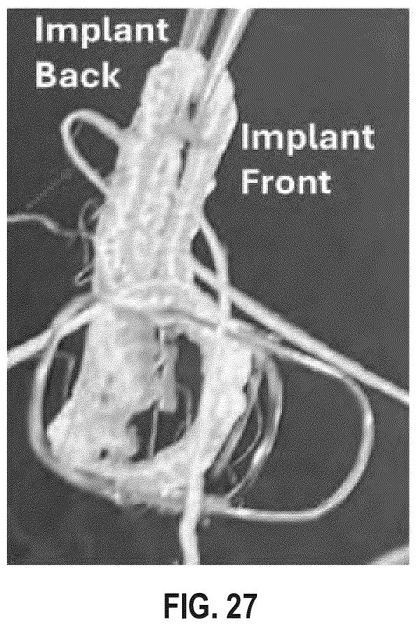

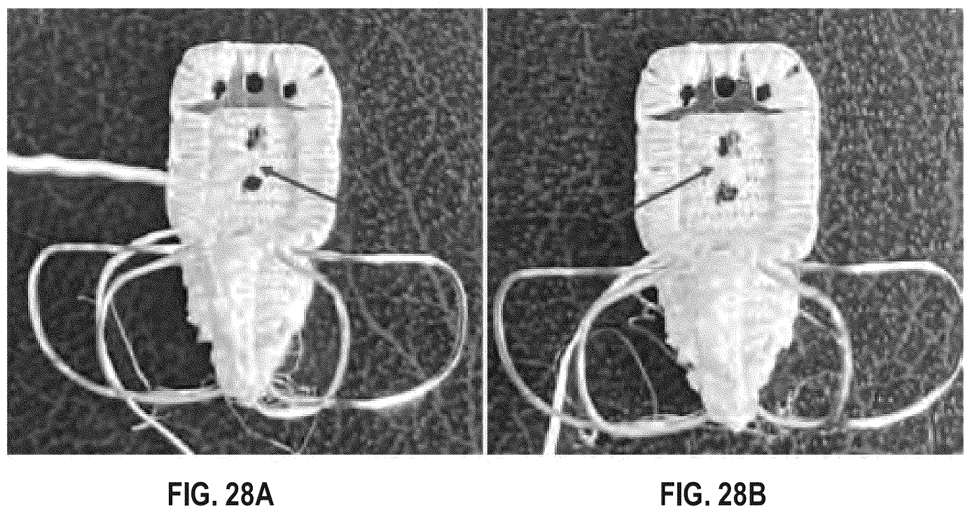

In some embodiments, a portion of the ventricular sock covering the tether holes 565 of the ventricular arm may be opened. For example, a cautery pen can be used to make holes 515 in the fabric of the ventricular sock to expose the tether holes 565 of the ventricular arm, as shown in FIGS. 26A and 26B. In some embodiments, a second suture may be fed through each of the tether holes from a back side of the implant, as shown in FIG. 27. In some embodiments, the suture loop of the second suture may be secured by placing a square knot on the front side of the implant (indicated by the arrow in FIG. 28A-28B). Knotting the second suture may further open the tether holes. In some embodiments, the knot of the second suture may be cauterized to flatten the knot into the ventricular arm. The second suture may be cauterized using a cautery tool and may be cauterized for less than a threshold amount of time to avoid burning of the fabric of the ventricular sock.

Once the ventricular sock is coupled to the ventricular arm, the method may include confirming the following: that the stitches around the perimeter of the ventricular sock are substantially straight and substantially even (e.g., same size), and without gaps between; that the ventricular sock is straight and symmetrical around the ventricular arm; that the tether holes are open on the ventricular arm without exposing bare metal; that the edges of the sock are free from burns from cauterizing the second suture, that the corners of the ventricular sock are wrapped around the spine; and that the six rows of friction elements protrude through the ventricular sock without interference from fabric or the stitches.

### Central Cover

In some embodiments, a central cover 522 may be coupled to the implant frame to cover at least the support members and central member. In some embodiments, the method may include cutting a piece of fabric into a shape including a first portion 532, a second portion 534, and a third portion 538. In some embodiments, the shape of the first portion 532 and the second portion 534 may correspond to a shape formed by the support members in an expanded configuration. In some embodiments, the first portion 532 and the second portion 534 may have a shape that is hexagonal (or oval, football shaped, or the like). In some embodiments, the third portion 538 may be substantially square or rectangular. In some embodiments, the first portion may have a top width X3 (e.g., between about 6 mm and 18 mm), a bottom width X4 (e.g., between about 8 mm and about 20 mm), a maximum width X5 (not shown) (e.g., between about 14 mm and about 26 mm), and a height Y3 (e.g., between about 7 mm and about 19 mm). In some embodiments, the second portion may have a top width X4, a bottom width X3, a maximum width X5, and a height Y3. In some embodiments, the third portion may have a height Y4 (e.g., between about 14 mm and about 26 mm) and a width X7 (e.g., between about 12 mm and about 24 mm).

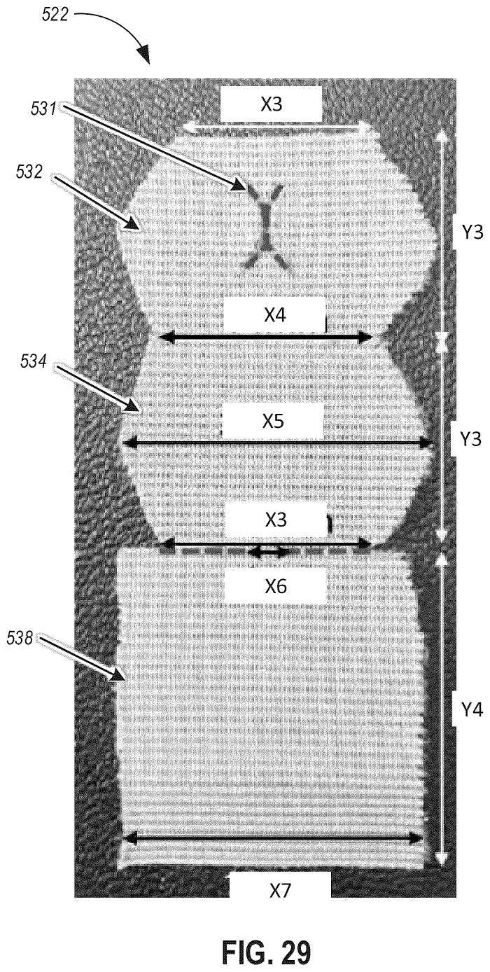

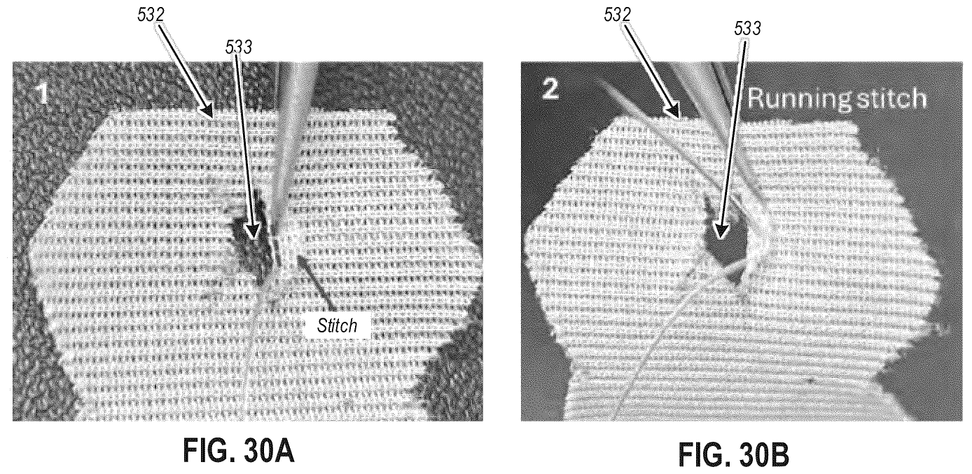

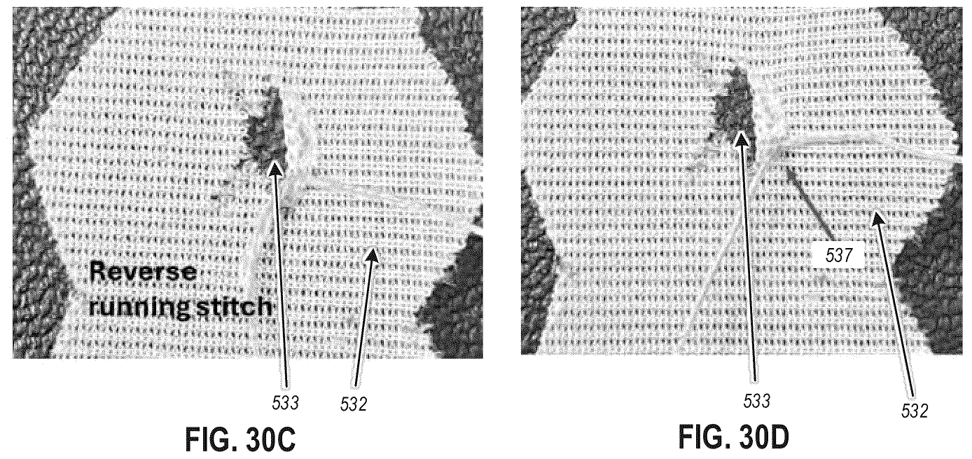

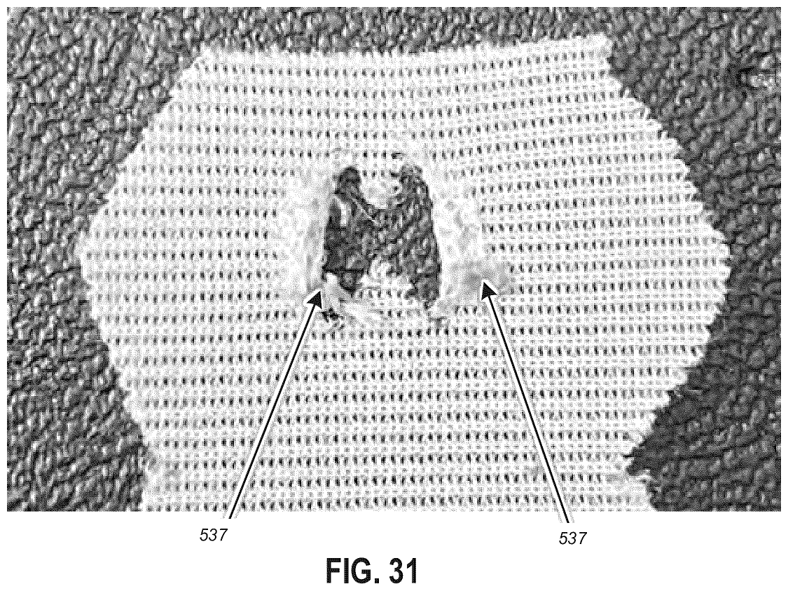

In some embodiments, the method may include cutting a pair of flaps 531 in a center region of the first portion 532 of the fabric (e.g., along the dotted lines shown in FIG. 29). In some embodiments, the method may include performing a running stitch from a bottom edge of a first flap toward the top edge of the first flap, as shown in FIGS. 30A-30B. Then perform a reverse running stitch to fill any gaps between stitches, as shown in FIG. 30C. In some embodiments, the stitching results in 3 to 6 stitches along each flap. In some embodiments, the method may include performing a square knot 537 with additional alternating knot at the bottom of each flap, as shown in FIG. 30D and FIG. 31. In some embodiments, the excess suture may be trimmed and cauterized to prevent fraying ends. In some embodiments, any or all of the stitching, reverse stitching, knotting, and cauterizing may be repeated for the second flap.

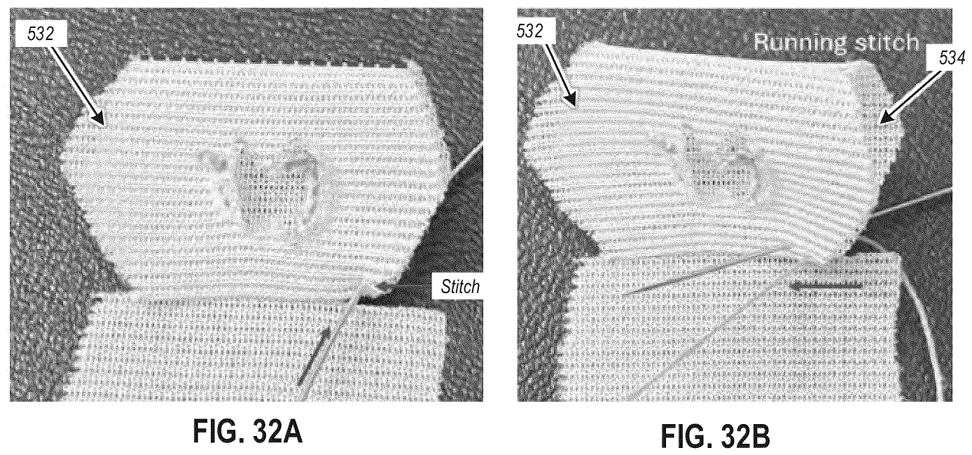

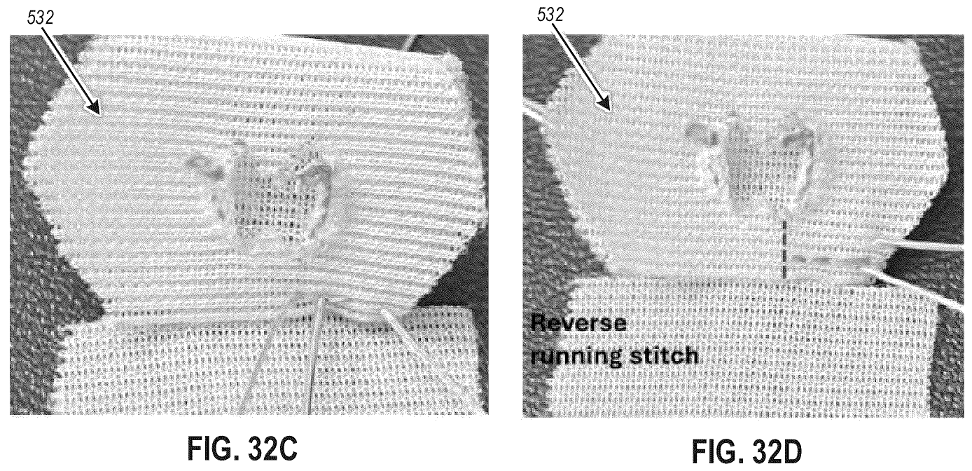

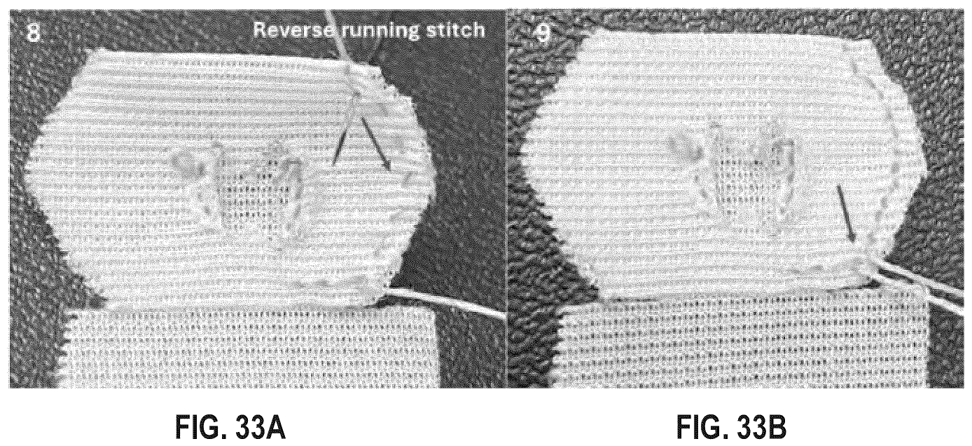

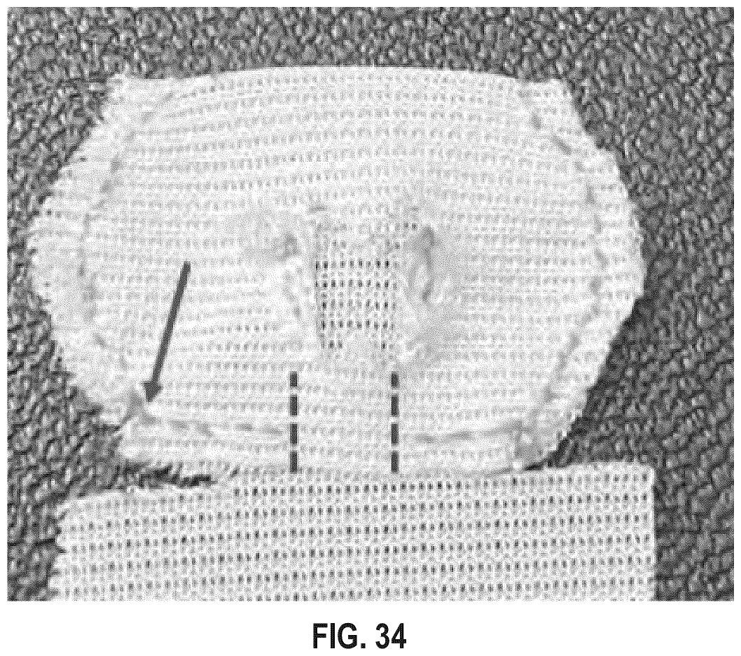

In some embodiments, the first portion of the fabric 532 may be folded over the second portion of fabric 534, as shown in FIGS. 32A-32D. For example, the fabric may be folded at the dotted line shown in FIG. 32A. In some embodiments, the suture knots from the flaps may face outward (e.g., away from the second portion of fabric). In some embodiments, a running stitch may be performed at a corner of the first fabric (e.g., a lower right corner shown by the arrow in FIG. 32A). In some embodiments, the method may include leaving a tail with the suture (e.g., 1-2 inches) to tie a knot when the stitching is complete. In some embodiments, the running stitch may be continued toward a centerline of the fabric, as shown in FIG. 32C. In some embodiments, the running stitch may stop when in line with the edge of the flap, as shown in FIG. 32D. The method may include performing a reverse running stitch toward the starting point (e.g., the corner). In some embodiments, a running stitch may be performed from the starting point toward a top corner of the fabric. In some embodiments, the stitch may follow a curvature of the fabric, as shown in FIG. 33A. In some embodiments, the method may include stitching over the folded and then reverse running stitch towards the starting point, as shown in FIG. 33B. In some embodiments, once the stitching is at the start point, the method may include tying a plurality of alternating knots. In some embodiments, the excess suture may be trimmed and cauterized. The steps of stitching the edge of the first portion and the second portion of fabric may be repeated for the second edge, as shown in FIG. 34. The stitching may leave an opening or gap that aligns with the flaps.

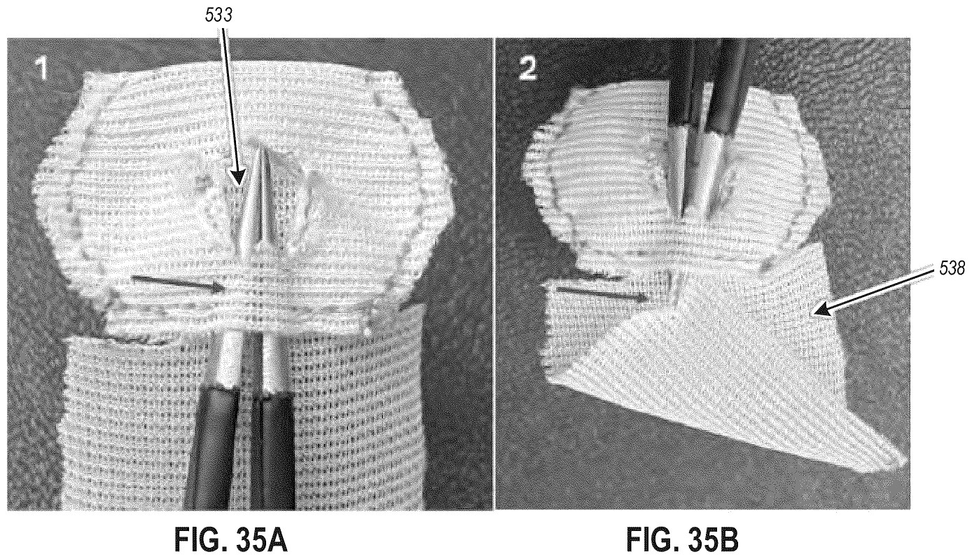

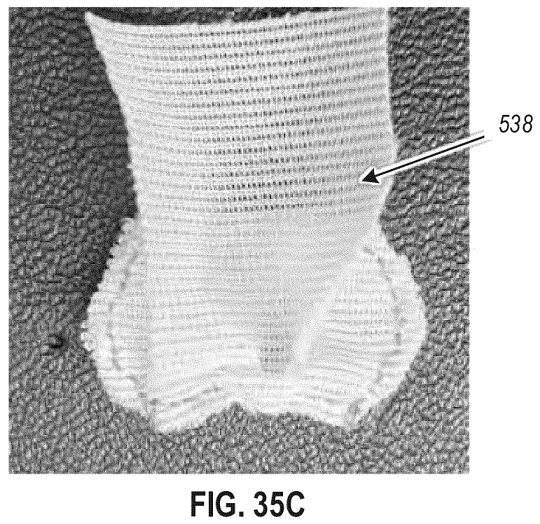

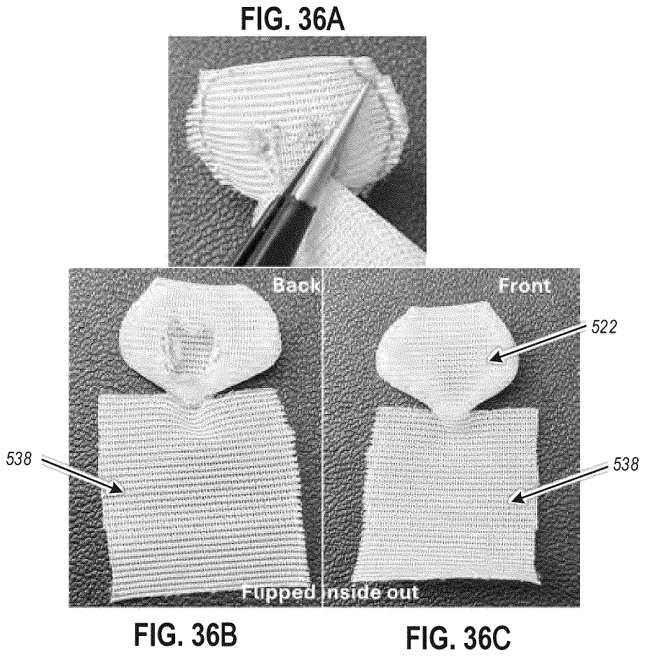

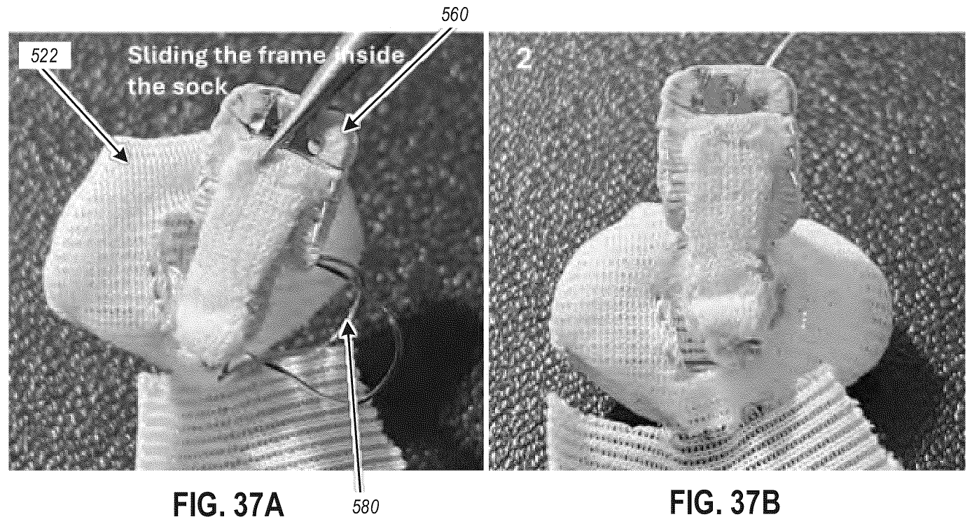

In some embodiments, the fabric may be flipped right side out, as shown in FIGS. 35A-35C. For example, forceps or tweezers can be used to grab one end of the third portion of fabric 538 and pull the third portion of fabric 538 through the opening 533 defined by the flaps. In some embodiments, the fabric may be flipped inside to form a pocket, and the suture seams are inside the pocket, as shown in FIG. 36. In some embodiments, the support members 580 of the implant 560 may be disposed inside the pocket formed by the central cover 522 via the opening defined by the flap, as shown in FIG. 37A. In some embodiments, the ventricular sock may be disposed on a side facing away from the central cover. In some embodiments, the remaining support members 580 may be pushed into an empty side of the central cover 522, as shown in FIG. 37B. In some embodiments, the support members 580 may be prevented from piercing the fabric during insertion into the pocket formed by the central cover 522.

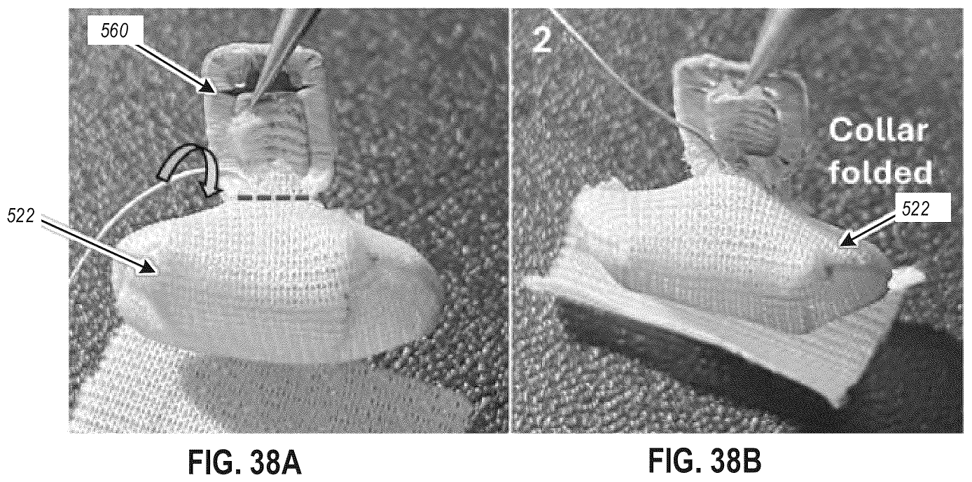

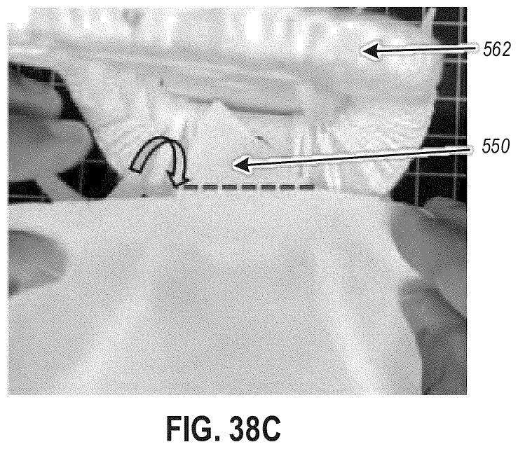

In some embodiments, once the support members are disposed in the pocket of the central cover 522, the central cover may be secured to the implant frame 560, as shown in FIGS. 38A-38C. In some embodiments, a triangular piece of the fabric 550 from the central cover 522 may be folded inwards where the atrial and ventricular arms meet the spine on the front-side of the support members, as shown in FIG. 38C.

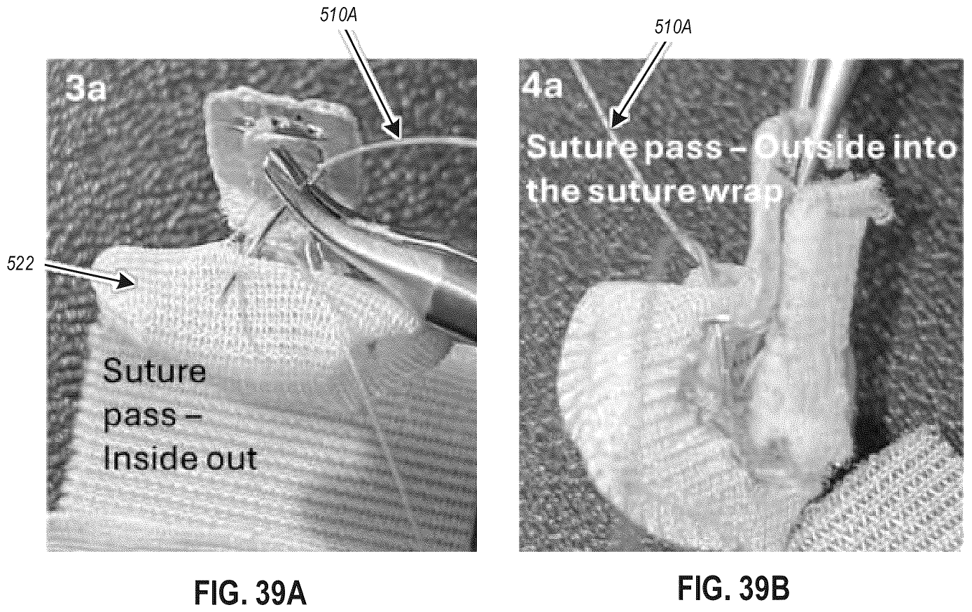

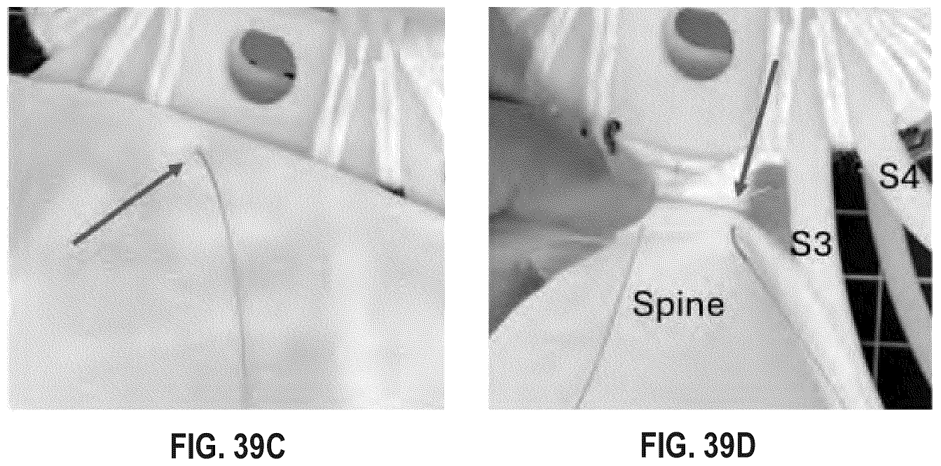

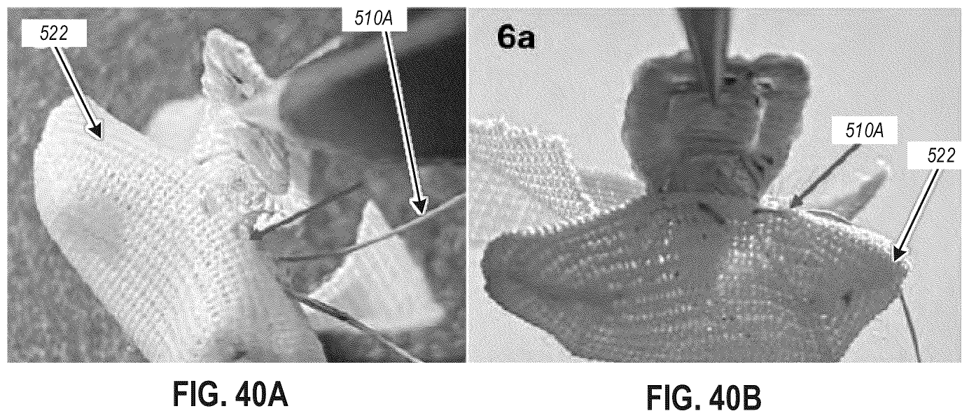

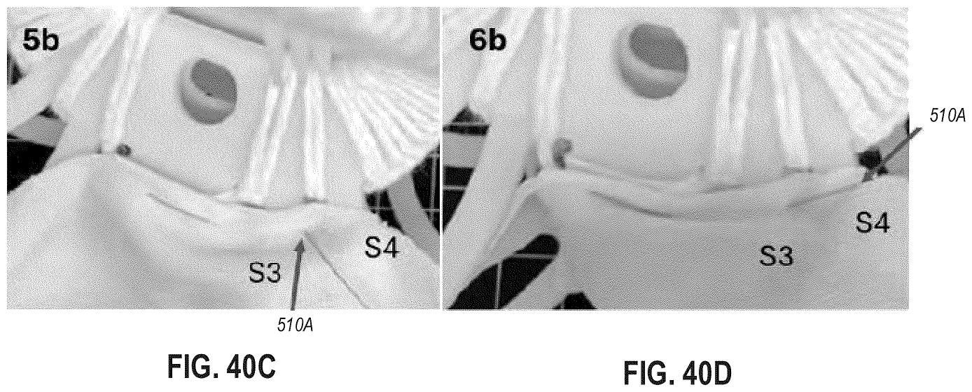

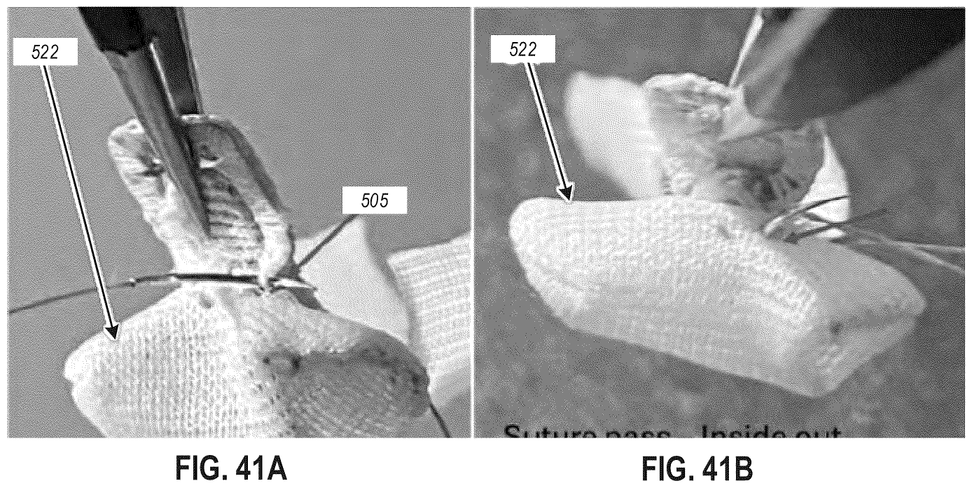

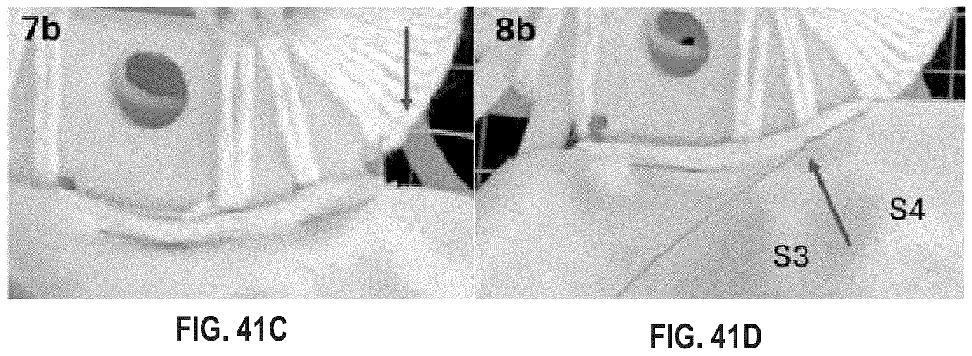

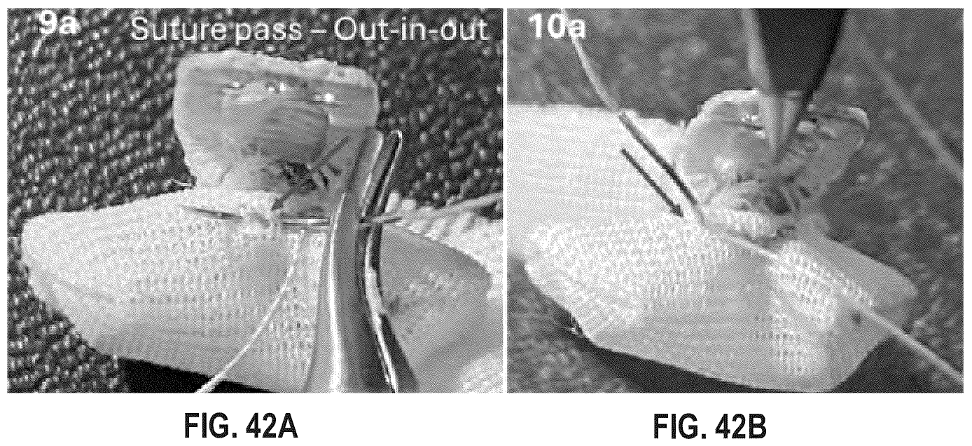

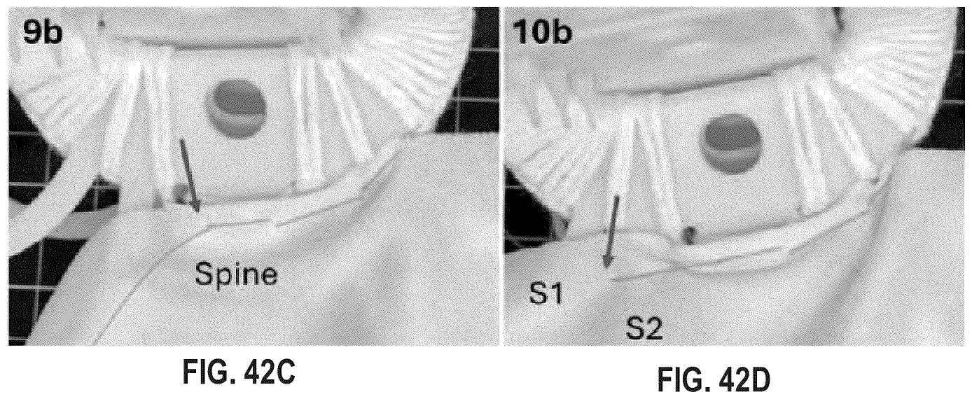

In some embodiments, the first suture end 510A may be (1) passed through the loop on the spine, through the triangular piece of fabric 550, and out of the central cover 522 towards a side of the spine, as shown in FIG. 39A and FIG. 39C. In some embodiments, the first end of the suture 510A may be (2) passed from the outside of the central cover 522, back into the fabric between the spine and the third support member S3, and through the suture wrap on the spine, as shown in FIG. 39B and FIG. 39D. In some embodiments, the first suture end 510A may be (3) passed from the inside of the pocket formed by the central cover 522, underneath the third support member S3 and back outside the pocket of the central cover 522 between the third support member S3 and the fourth support member S4, as shown in FIG. 40A and FIG. 40C. The first suture end 510A may be (4) passed over the fourth support member S4, and into the pocket of the central cover 522 outside of the fourth support member S4, as shown in FIG. 40B and FIG. 40D. In some embodiments, the needle 505 may be (5) passed into the suture wrap with the needle facing away from the implant, as shown in FIG. 41A and FIG. 41C. The needle 505 may be (6) passed under the fourth support member (location denoted by S4, support member not shown) and back to the outside of the central cover 522 between the fourth support member S4 and the third support member S3, as shown in FIG. 41B and FIG. 41D. In some embodiments, a stitch can be (7) formed over the third support member S3. For example, the needle can pass back into the fabric between the third support member S3 and the spine, pass over the spine, through the suture wrap on the spine, and out on the left side of the spine, see FIG. 42A and FIG. 42C. In some embodiments, the stitch formed in FIG. 42A and FIG. 42C may be formed under the fabric of the central cover and above the spine of the implant. The steps (1)-(7) create a running stitch over and under the support members on one side.

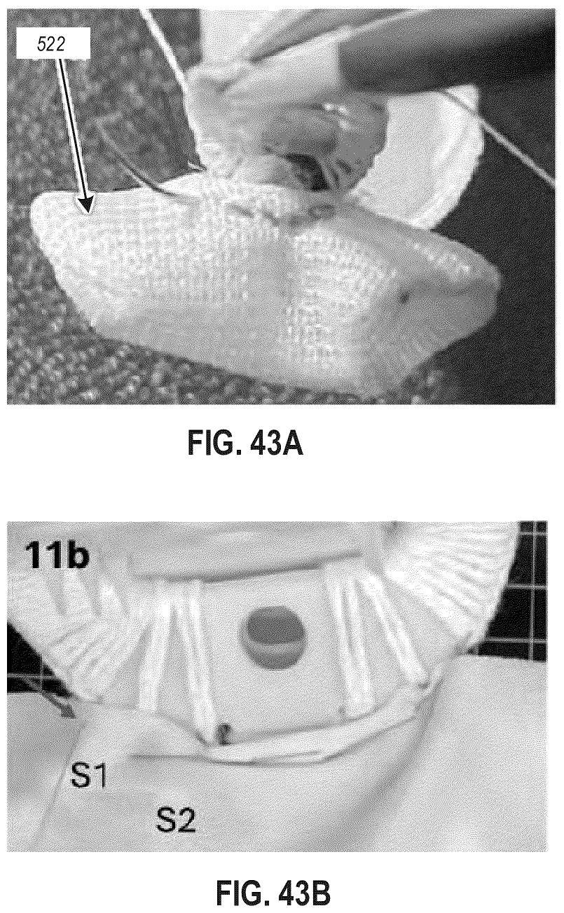

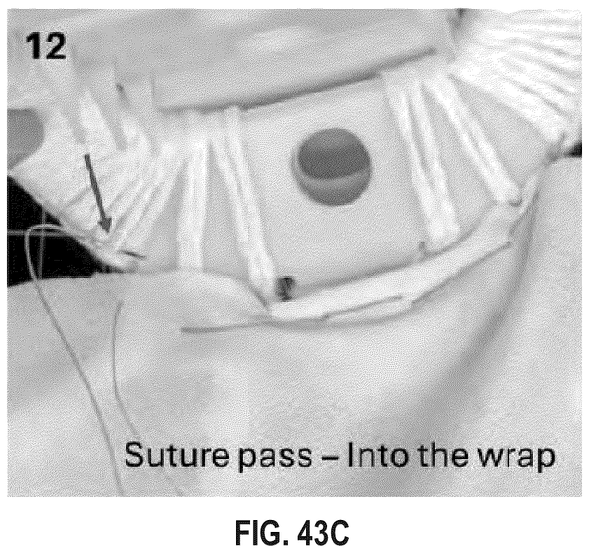

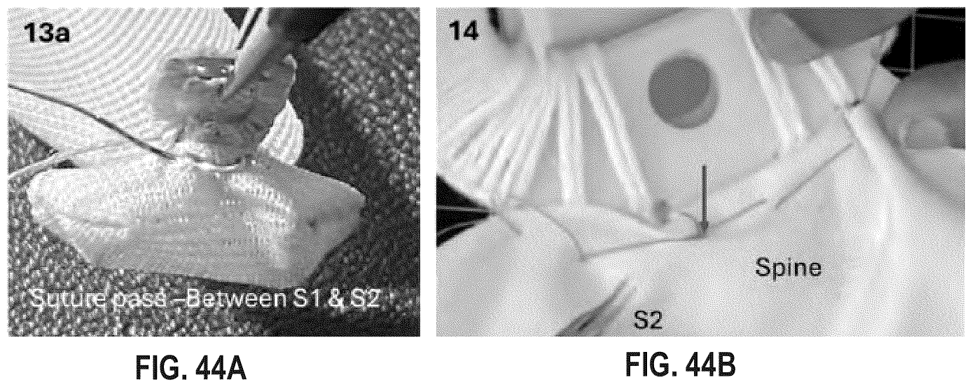

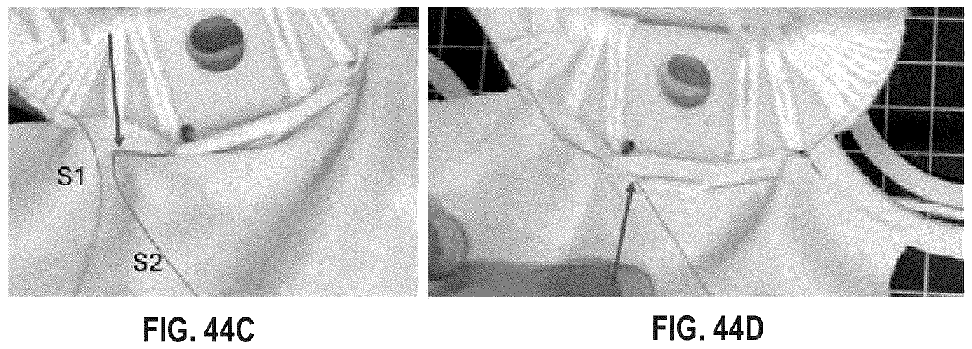

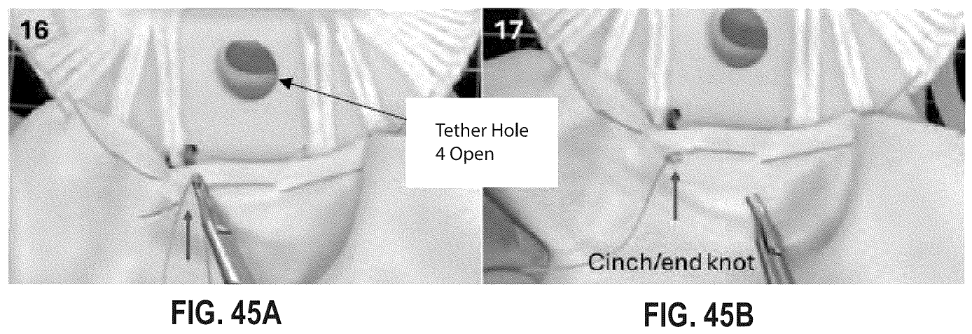

In some embodiments, the first end of the suture may be passed over the second support member S2 and into the fabric to the inside of the central cover pocket 522 between the second support member S2 and the first support member S1, as shown in FIG. 42B and FIG. 42D. The first end of the suture may be passed underneath the first support member and out through the central cover pocket 522, as shown in FIG. 43A and FIG. 43B. In some embodiments, the first suture end may be passed into a suture wrap on the atrial arm (e.g., with the needle facing towards the implant), as shown in FIG. 43C. In some embodiments, the needle may be passed over the first support member S1 and back into the fabric between the first support member S1 and the second support member S2, as shown in FIG. 44A and FIG. 44C. The first end of the suture may be passed outside of the central cover pocket and between the second support member S2 and the spine, as shown in FIG. 44B and FIG. 44D. The suture may be passed into the suture wrap on the spine and out of the central cover pocket towards a side of the spine (e.g., the left side when the implant is facing upwards), as shown in 45A. In some embodiments, the first suture end may be cinched, or an end knot may be performed, as shown in FIG. 45B.

In some embodiments, the method can include verifying any or all of the following: there are a predetermined number of visible stitches on the outside securing the central cover over and under the support members of the frame and/or that there is one stitch over and one stitch under each support member.

### Pillow

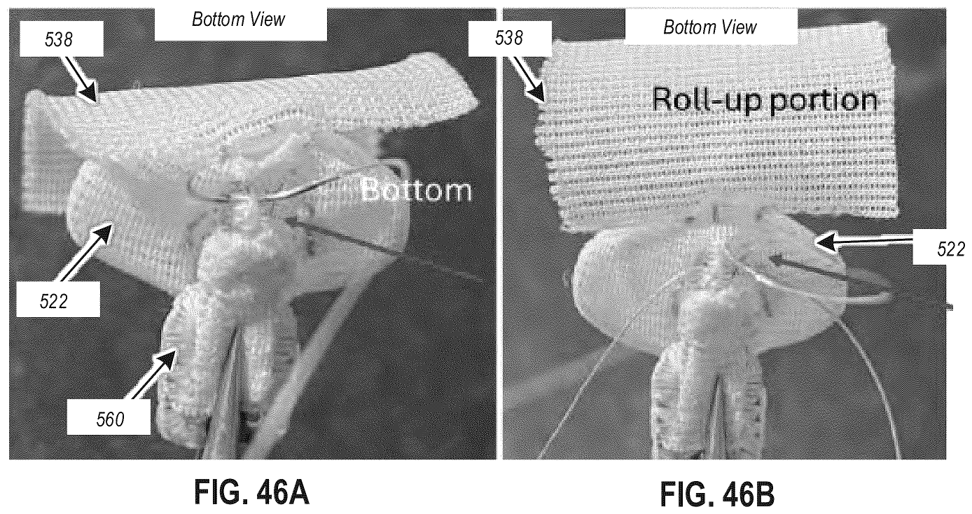

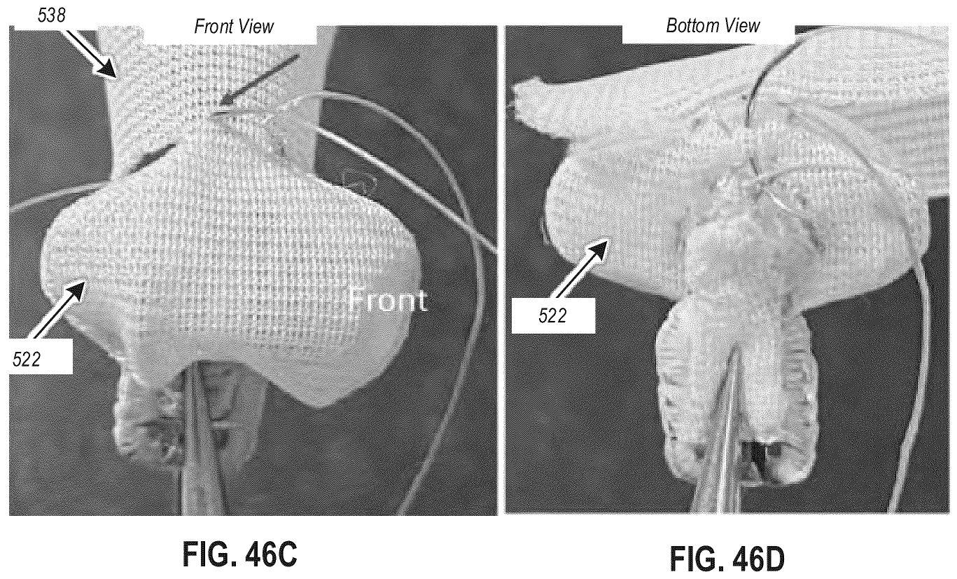

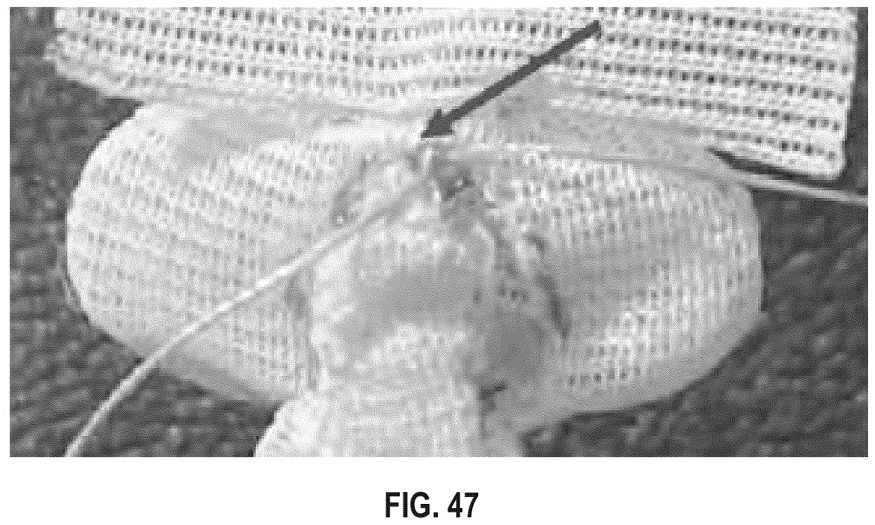

In some embodiments, once the pocket of the central cover 522 is secured to the implant frame 560, a pillow may be formed using the third portion of fabric 538 of the central cover 522. In some embodiments, the method may include passing a needle (e.g., a straight needle) through the bottom of the spine as shown in FIG. 46A. In some embodiments, the needle may be passed into the suture wrap (e.g., a suture wrap on the spine), through the triangular fabric flap, and through the third portion of the fabric 538 to close the gap between the pocket of the central cover 522 and the third portion of fabric 538, as shown in FIG. 46B. Then the needle can be passed back through the rectangle piece and through the bottom of the central cover 522 near the first needle pass and back into the suture wrap, as shown by FIG. 46C. In some embodiments, the suture can be secured with three alternating knots (e.g., 1 square knot with an additional alternating knot,) as shown in FIG. 47. In some embodiments, the suture may be left with at least one tail or free end. In some embodiments, the tail may not be cut or cauterized.

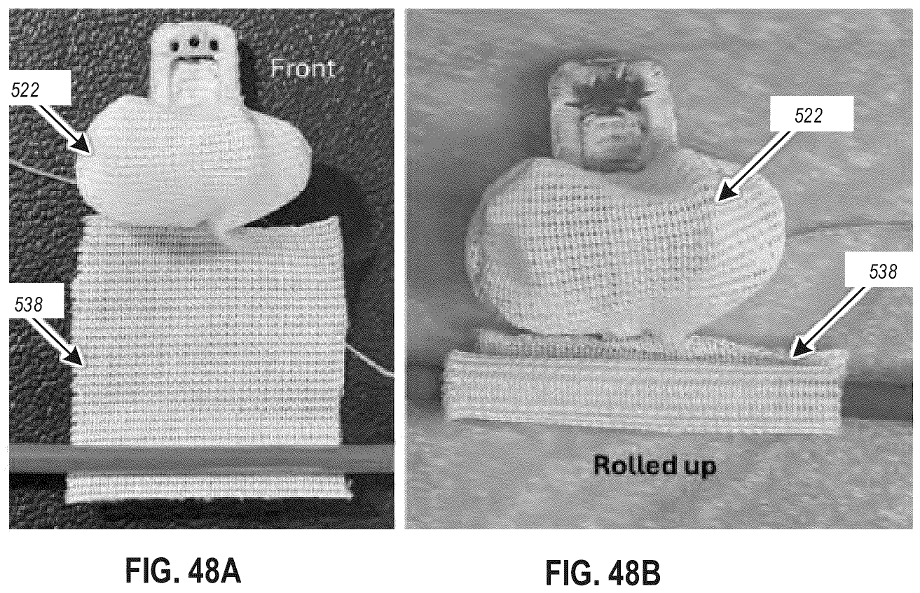

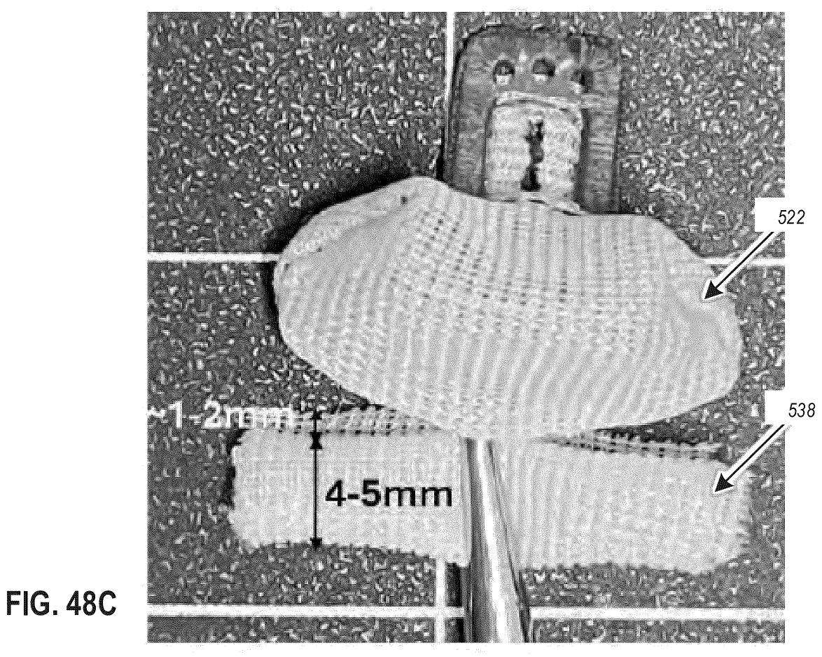

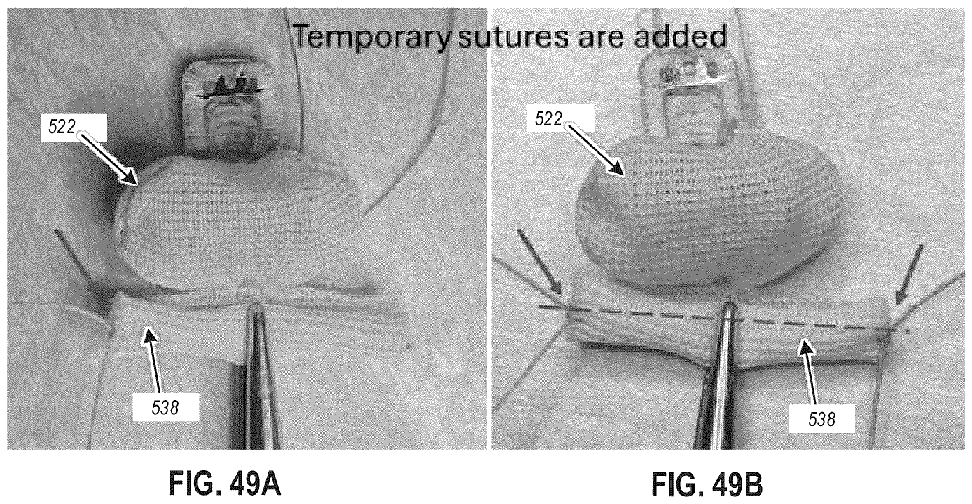

In some embodiments, the implant may be oriented with the front side facing upwards, and the third portion of fabric 538 may be rolled using a mandrel towards the support members and central cover 522, as shown in FIGS. 48A-48B. In some embodiments, the third portion of fabric 538 may be rolled such that a section of the third portion is left unrolled (e.g., about 1-2 mm of fabric). In some embodiments, the mandrel may be removed while the fabric is held with a tool (e.g., a hemostat, grasper, tweezers, or the like), as shown in FIG. 48C. In some embodiments, the rolled-up portion may have a predetermined outer diameter. For example, the rolled-up portion may have an outer diameter of about 4-5 mm.

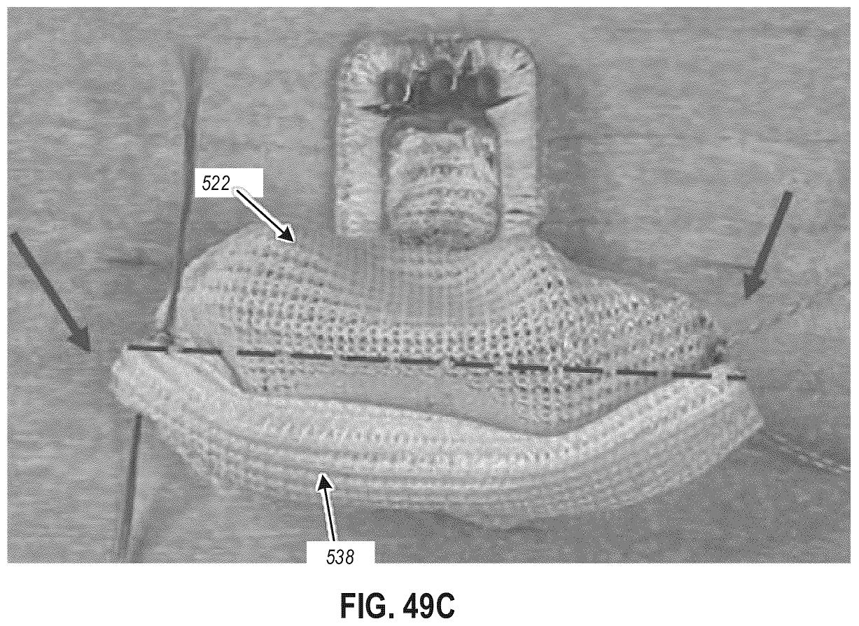

Once the third portion of fabric 538 is rolled, each end of the rolled-up portion of fabric may be temporarily tacked (e.g., using a temporary suture) to the central cover 522 to form the pillow 540. In some embodiments, each end of the rolled-up portion may be temporarily tacked about midway up (corresponding to the widest portion) of the central cover, as shown in FIG. 49C. In some embodiments, an edge of the pillow may be sutured to the central cover 522.

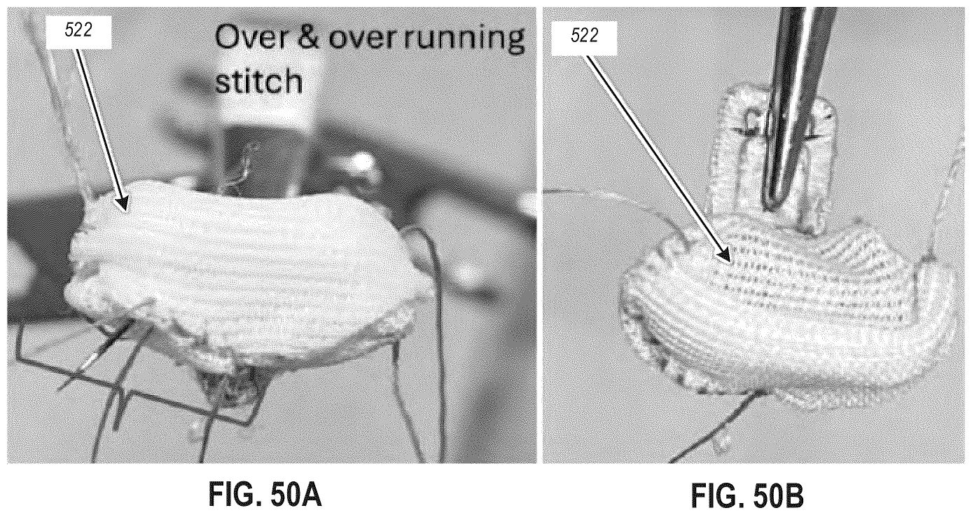

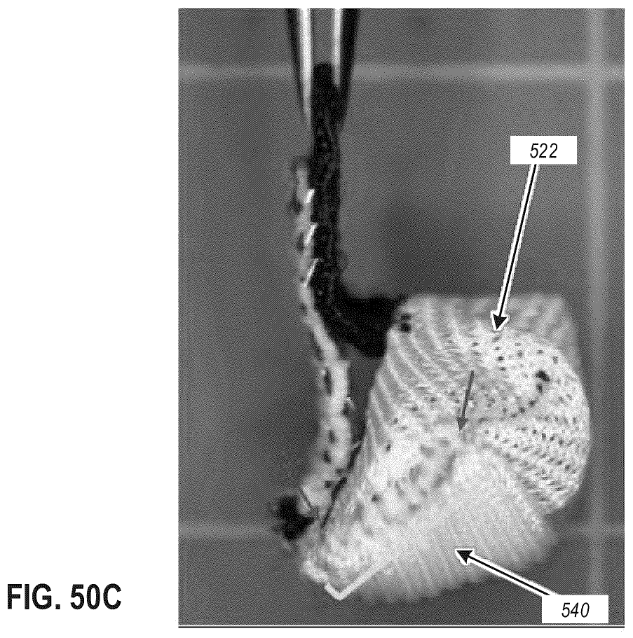

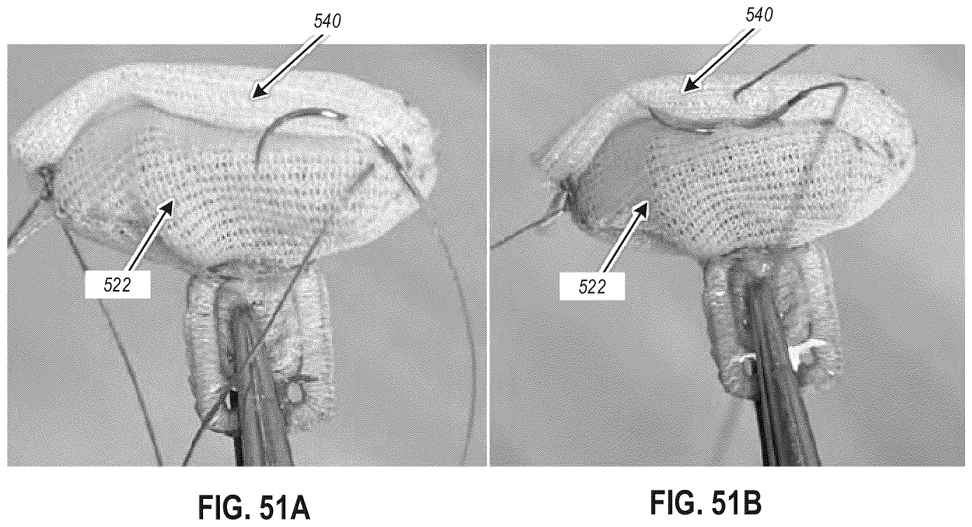

In some embodiments, the suture secured to the bottom of the spine of the implant frame may be used to secure the pillow 540 to the pocket of the central cover 522. In some embodiments, each short side of the pillow 540 may be secured to the pocket of the central cover 522, as shown in FIGS. 50A-50C. In other words the corners of the pillow may be secured to the central cover such that the edges of the pillow are atraumatic (e.g., the pillow does not include rough edges). In some embodiments, the rolled-up portion of the fabric may be shaped such that the pillow includes corners, ears, extensions, flaps, or the like (not shown) that extend out from the sides of the coaptation surface.

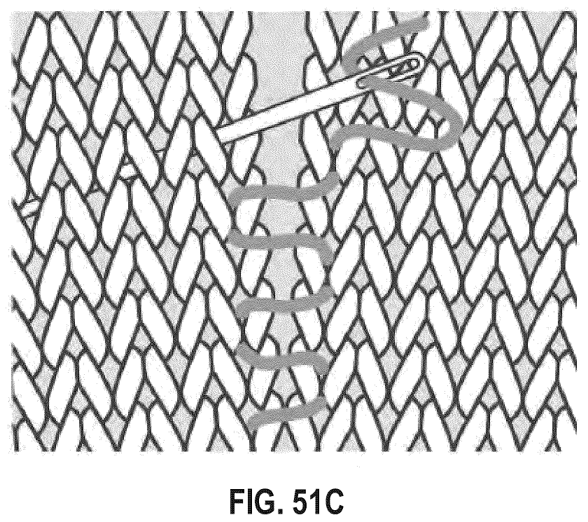

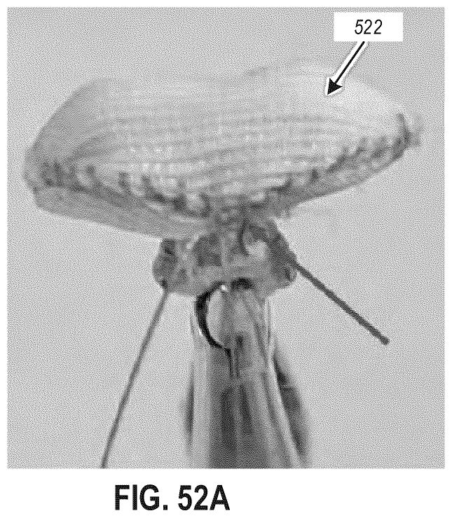

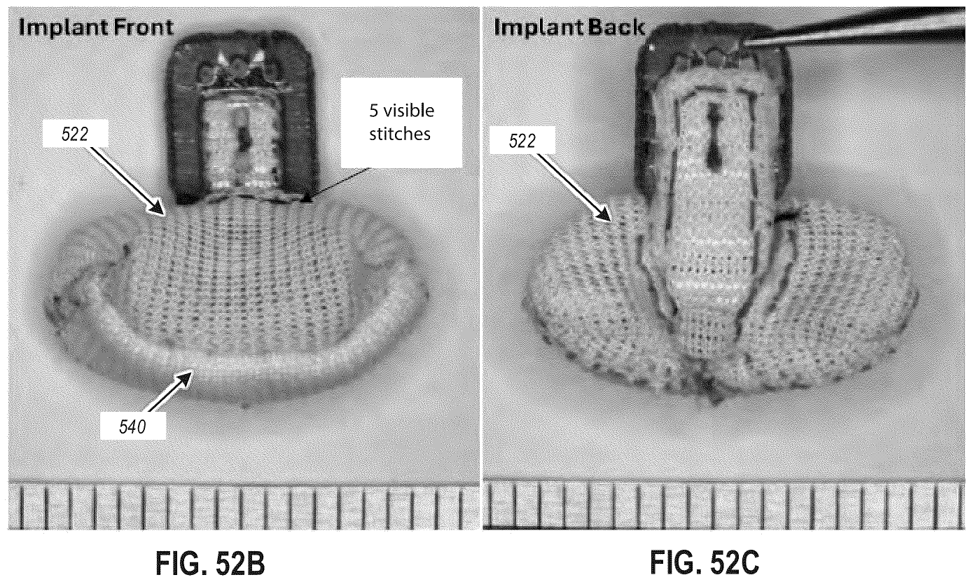

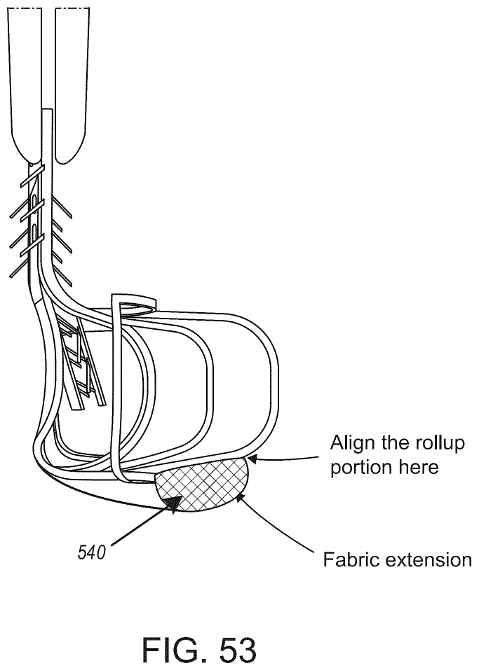

In some embodiments, a top portion of the pillow (e.g., a portion of the pillow on the front side of the implant) may be stitched, shown in FIGS. 51A-51B. In some embodiments, a mattress stitch (shown in FIG. 51C) may be performed along the top portion of the pillow to secure the pillow to the central cover. In some embodiments, the stitching may secure the pillow to a lower curvature of the support members, as shown in FIG. 52A. In some embodiments, the stitching may be performed from a first corner of the pillow towards a second corner of the pillow. Once the second corner is reached, the stitching may switch back to an over and over stitching, as shown in FIGS. 52A-52C. In some embodiments, the temporary sutures (e.g., the sutures to temporarily tack the corners of the pillow) may be removed. In some embodiments, the method includes suturing from the second corner back to the center point (shown with the arrow in FIG. 52A). In some embodiments, the excess suture can be trimmed and cauterized. In some embodiments, the pillow may be symmetrical. In some embodiments, edges of the rolled-up portion of fabric may align with a lower coverture of the support members, as shown in FIG. 53.

In some embodiments, the method can include verifying any or all of the following: the pillow is symmetrical; the pillow is along the bottom of the implant next to the radiused edge; that the stitches on the bottom of the pillow are secure and tight without gaps; that the corners of the rolled-up portion are secured to the support members; that the knots are secure.

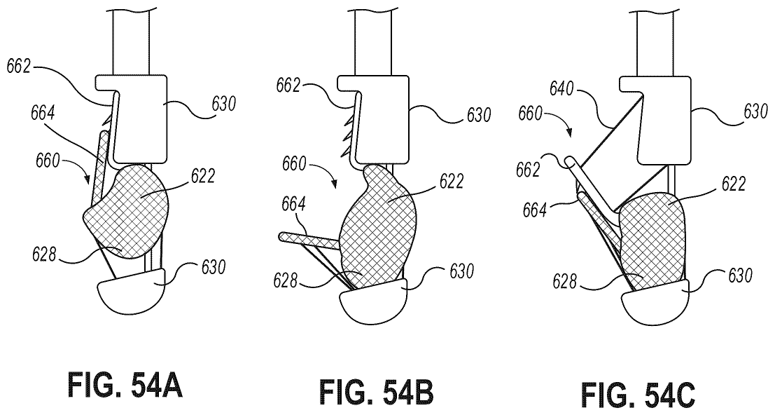

FIGS. 54A-54B show an implant 660 including a cover coupled to an implant holder 630 in a closed configuration and an open configuration, respectively, according to an embodiment. The cover includes a ventricular sock disposed over the ventricular arm 664, a central portion 622 disposed around the support members (not shown), and a pillow 628 extending from a distal end of the implant 660. The central portion 622 of the cover can be coupled to a suture wrapped around the atrial arm 662. As shown in FIG. 54A, the atrial arm 662 and the ventricular arm 664 of the implant abut a portion of the implant holder 630, and the central cover 622 encloses or covers a portion of the implant 660 extending distally from the atrial arm 662 and the ventricular arm 664. In the open configuration, the ventricular arm 664 extends laterally away from the atrial arm 662. As shown, the pillow 628 forms an enlarged portion at the distal end of the implant 660, which can improve coaptation when the implant 660 is implanted. FIG. 54C shows the implant 660 loosened away from the implant holder 630. Tension can be removed from tethers 640 coupling the atrial arm 662 and the ventricular arm 664 such that the implant 660 moves away or is loosened away from the implant holder 630.

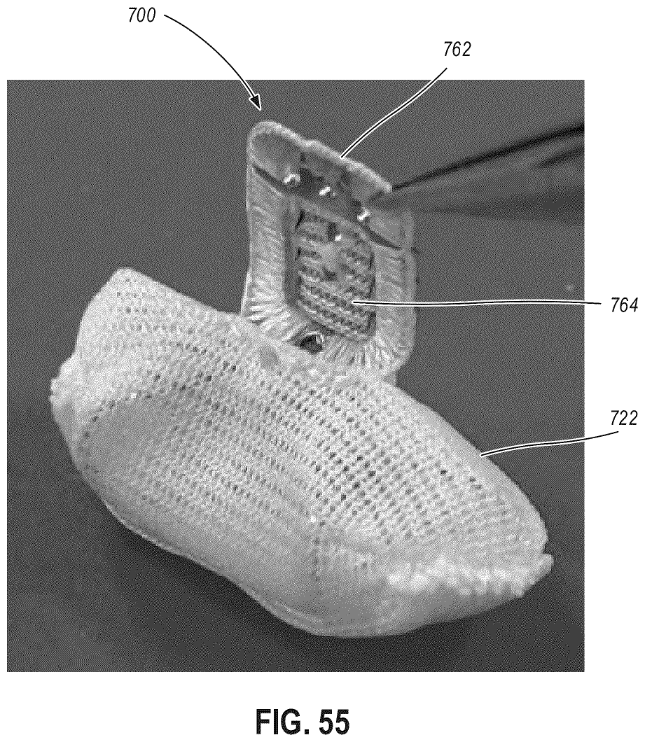

FIG. 55 shows an implant 700 including an implant frame covered with a cover including a central cover and a suture to protect the implant frame during implantation, according to embodiments. The implant 700 includes an atrial arm 762, a ventricular arm 764, and a plurality of support members (not visible). In some embodiments, the atrial arm 762 of the implant 700 is wrapped in a suture, and the ventricular arm 764 is covered with fabric (i.e., a ventricular sock). The plurality of support members are covered with a central cover 722 to form a coaptation portion of the implant that is configured to make contact with a portion of a leaflet of the heart valve to prevent heart valve regurgitation. In some embodiments, a shape of the coaptation section can be adjusted based on a shape of the central cover 722. For example, the central cover 722 can have one or more extensions to increase likelihood of coaptation with a leaflet of the heart valve (e.g., and reduce a gap between the leaflets), as described in further detail below.

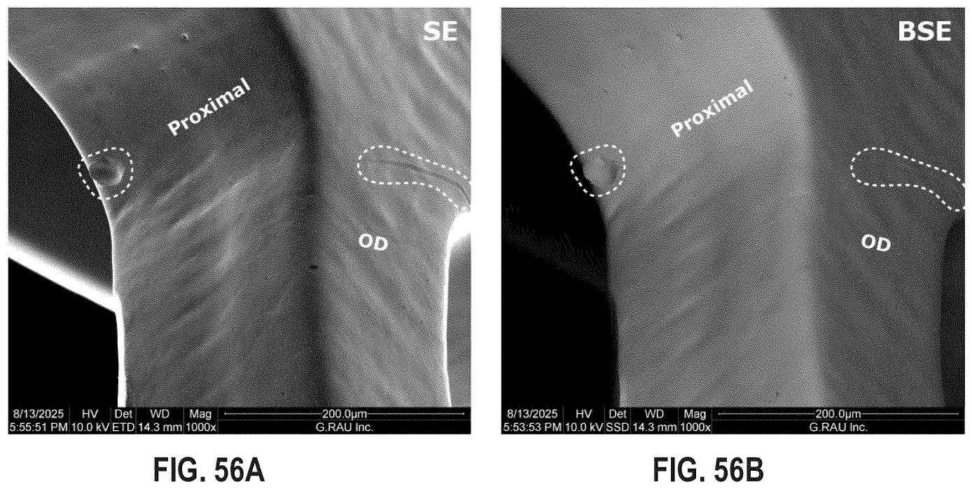

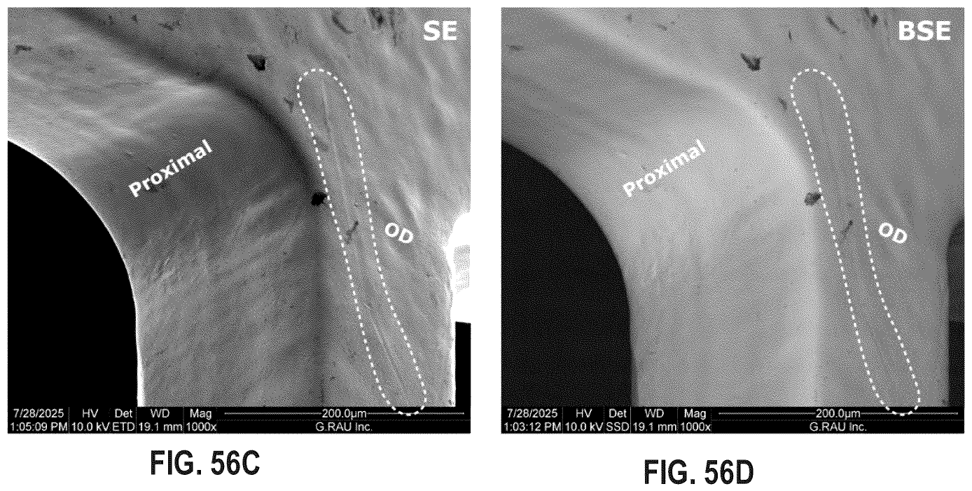

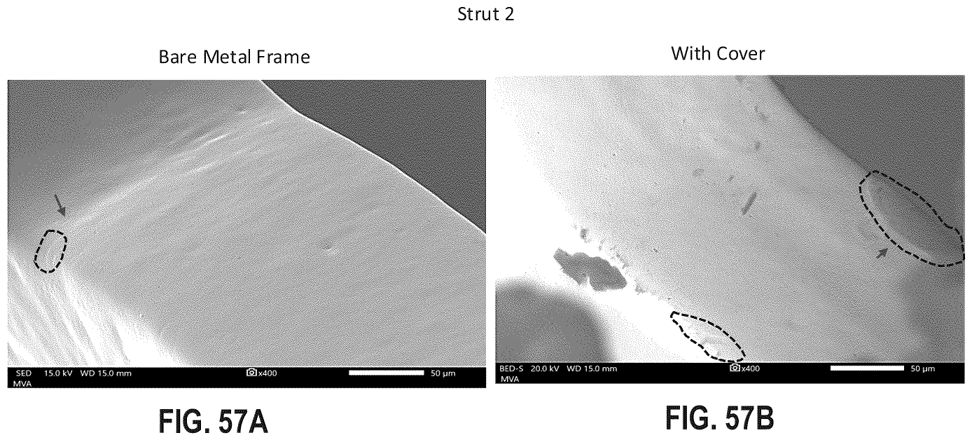

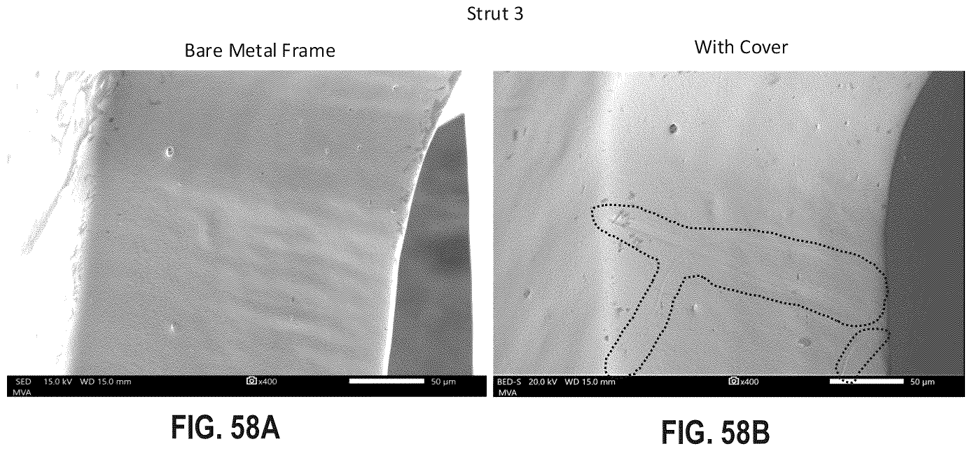

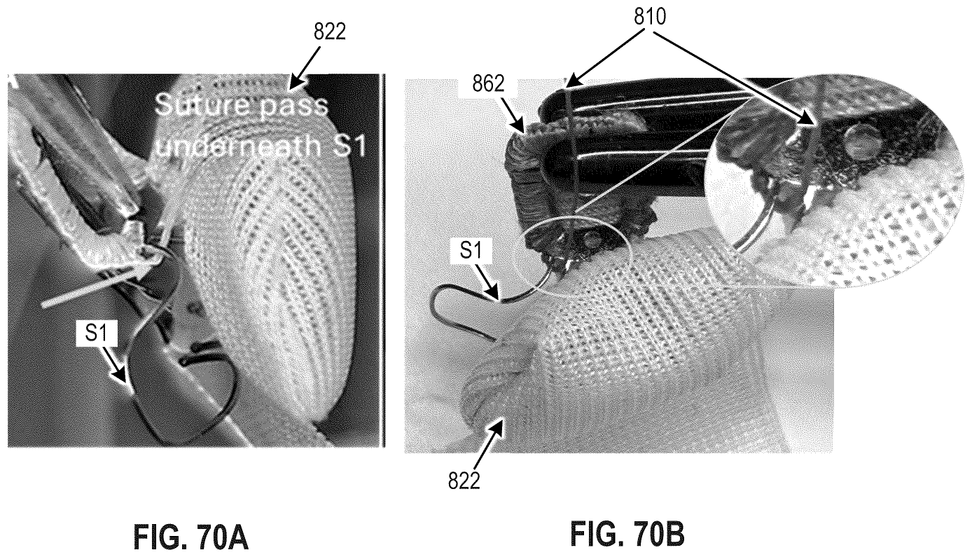

FIGS. 56A-58D show close-up images of stress that occurs on the support members of the implant frame due to deformation during covering the implant frame. FIGS. 56A-56B shows defects (e.g., craters or cracks) that form in a material of the third support member (e.g., S3 shown in FIGS. 4C-4D), particularly at a transition point between the atrial arm and the third support member. FIGS. 56C-56D show defects that form in a fourth support member (e.g. S4 shown in FIGS. 4C-4D) at the transition point between the atrial arm and the fourth support member. FIGS. 57A and 58A show a surface of the second support member (e.g., S2 shown in FIGS. 4C-4D) and the third support member, respectively, having less defects before deformation that can occur during covering the implant. FIGS. 57B and 58B show the second support member and the third support member when the implant frame is deformed during covering of the implant. The embodiments described herein address this problem by reducing deformation of the support members during installation of the cover on the implant frame. For example, the central cover may be prepared such that the support members are not deflected when disposed in the central cover (e.g., as shown in FIGS. 70A-75). Additionally, in some embodiments, the central cover may be partially attached to the frame prior to full securement to allow the user to see the support members when wrapping the suture therebetween, thereby reducing risk of deformation.

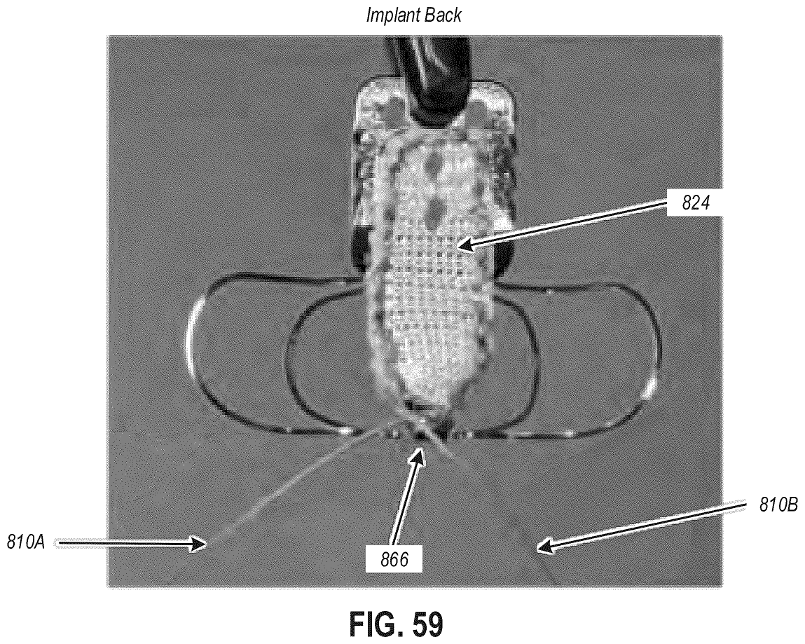

FIGS. 59-75 outline steps of an example method of covering the implant frame, according to embodiments. The method shown in FIGS. 59-75 can be similar to the method shown in FIGS. 6A-52C, and therefore, certain aspects of the method are not described herein again with respect to FIGS. 59-75. Any of the steps described herein with reference to FIGS. 59-75 can be performed in place of or in addition to the steps described with respect to FIGS. 6A-52C. All such variations are within the scope of this application.

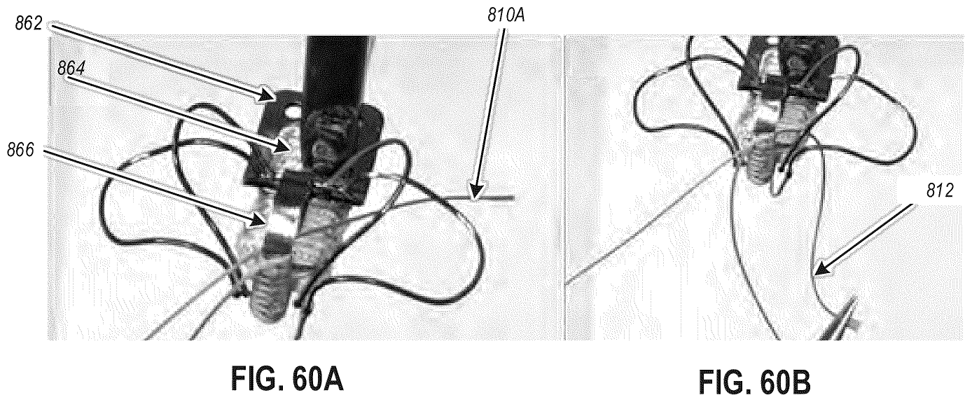

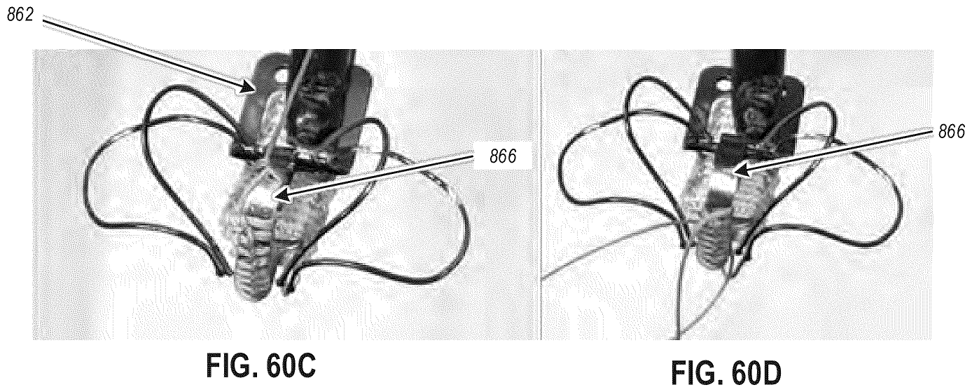

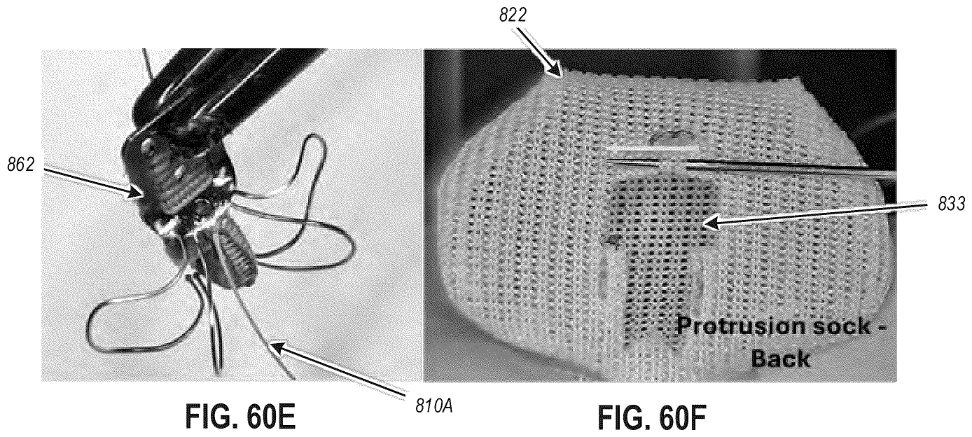

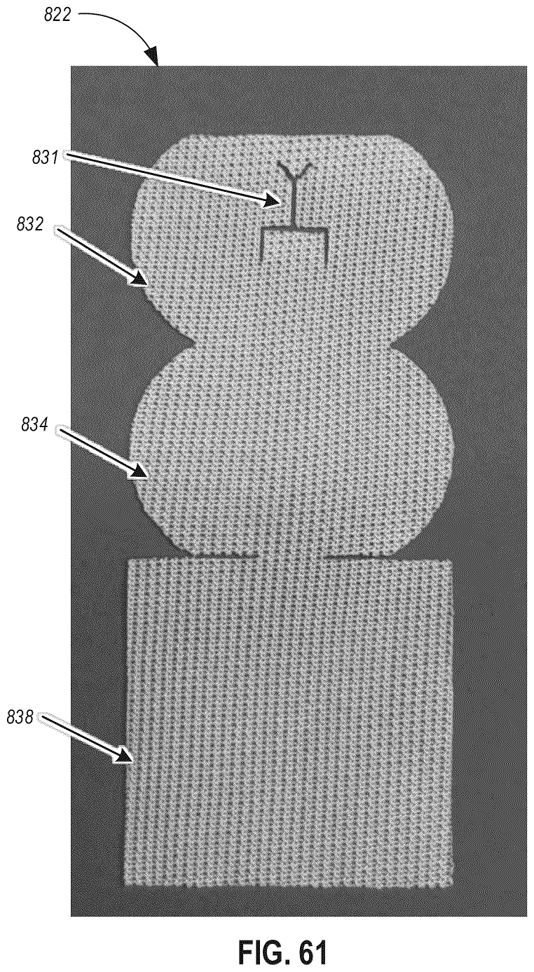

To couple a cover to the implant, a portion of the implant is wrapped in a suture. In some embodiments, the implant may be at least partially wrapped with a suture to protect the portion wrapped and/or to provide an anchor for coupling one or more covers or portions of a cover to the implant. As shown in FIGS. 59-60D, the suture 810 may be secured (e.g., knotted) to the spine 866 of the implant leaving a first free end (hereinafter, “the first end”) 810A and a second free end (hereinafter, “the second end”) 810B. In some embodiments, the first end 810A may be longer than the second end 810B (e.g., similar to the first end 510A and the second end 510B). In some embodiments, the suture may be secured to the spine using a square knot and a left-right-left knot (i.e., 3 alternating knots). FIGS. 19A-19K show steps of forming the knot to secure the suture to the spine of the implant and are described above.

Referring to FIGS. 60A-60D, the suture can be secured to the spine 866 near a base of the ventricular arm (not shown). After the suture is fixed to the spine 866, the method may include wrapping the suture around the spine 866 towards the atrial arm 862. The wrapping of the suture may include disposing the first end 810A of the suture under the spine 866 (e.g., when the implant is oriented with the atrial side up), as shown in FIG. 60A. The method can include pulling the first end 810A of the suture back over a top of the spine to form a loop 812, and threading the first end 810A of the suture through the loop 812, as shown in FIG. 60B. The method may include tightening the loop 812 so that the suture lies snug against the spine 866, as shown in FIG. 60C. The method may include repeating the steps of (i) disposing the suture under the spine; (ii) pulling the first end 810A of the suture back over the top of the spine; (iii) threading the first end 810A of the suture through a loop 812; and (iv) tightening the loop 812 such that the suture wraps around the implant spine 866. In some embodiments, after each loop is tightened to form a portion of the suture wrap, the method may include sliding a respective loop to contact the previously formed loop to reduce gaps between adjacent suture loops, as shown in FIG. 60D. In some embodiments, the suture may be wrapped around the spine 866 until the spine 866 is at least partially covered. In some embodiments, the suture 810 may be wrapped around the spine 866 until the spine 866 is fully covered. In some embodiments, the suture may be wrapped around the spine 866 until a suture loop 812 sits at a base of the atrial arm 862, as shown in FIG. 60E. As shown, the first end 810A of the suture is free for subsequent wrapping of the implant frame. In some embodiments, the spine of the implant can be wrapped after the ventricular arm is covered. For example, as shown in FIG. 59A, a portion of the suture used to attach the ventricular sock to the ventricular arm 864 can be used to wrap the spine 866 of the implant. In some embodiments, the ventricular arm can be covered according to the steps shown in FIGS. 3B and 16-28B.

In some embodiments, the central cover 822 may be coupled to the implant frame to cover at least the support members and central member. As shown in FIG. 60E, a central cover 822 (also referred to herein as a protrusion sock) can be at least partially attached to the implant via the free end of the first suture 810A (e.g., after the implant spine is wrapped and before the atrial arm 862 is wrapped). For example, the free end of the suture 810A can be woven through a back side of the central cover 822 such that an implant spine is aligned with an opening 833 formed in the back side of the central cover 822.

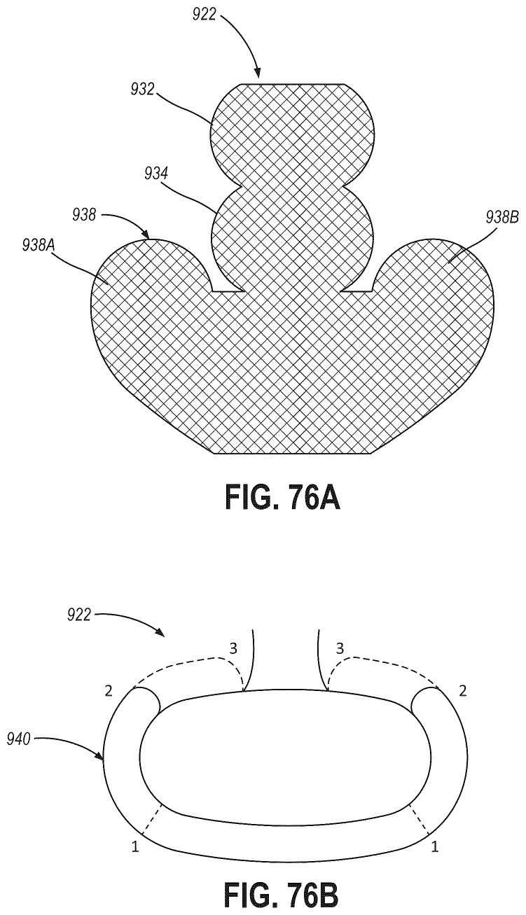

In order to form the central cover 822, the method may include cutting a piece of fabric into a shape including a first portion 832, a second portion 834, and a third portion 838, as shown in FIG. 61. In some embodiments, the shape of the first portion 832 and the second portion 834 may correspond to a shape formed by the support members in an expanded configuration. In some embodiments, the first portion 832 and the second portion 834 may have a shape that is oval-like (or any other suitable shape). In some embodiments, the third portion 838 may be substantially square or rectangular. In some embodiments, the third portion 838 can have a shape configured to form a volume that corresponds to a geometry of a portion of the heart valve of the patient. For example, the third portion 838 can have lateral extensions extending therefrom (e.g., as shown in FIGS. 76A-79D) to increase a coaptation area of the central cover 822 when the third portion 838 is formed into the central cover 822. In some embodiments, dimensions of the first, second, and third portions 832, 834, 838 can be substantially similar to the dimensions described with respect to FIG. 29.

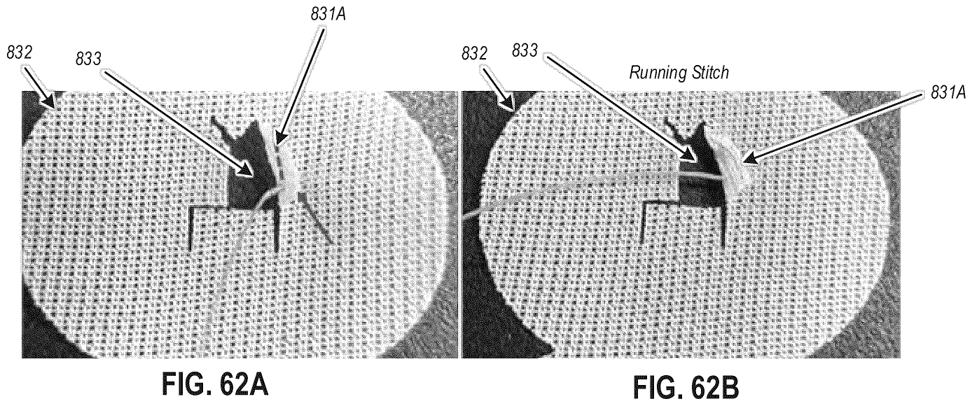

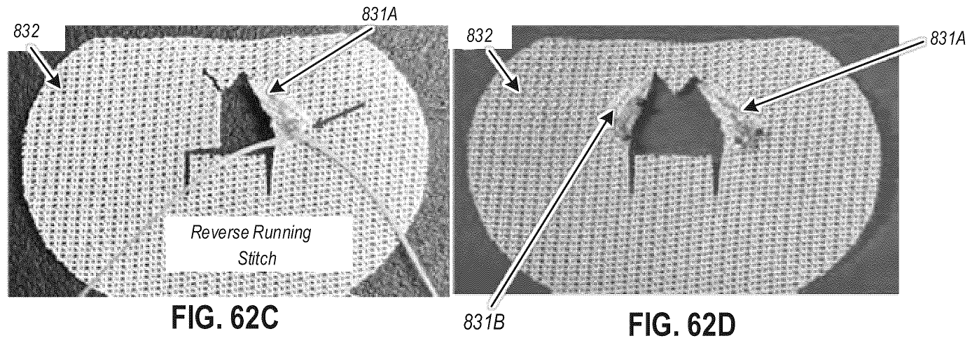

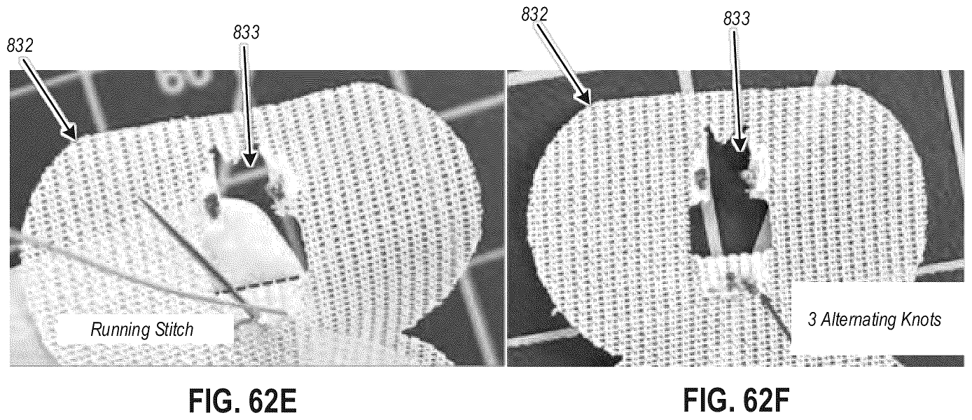

In some embodiments, the method may include cutting a pair of flaps 831 in a center region of the first portion 832 of the fabric. In some embodiments, the method may include performing a running stitch from a bottom edge of a first flap 831A toward the top edge of the first flap, as shown in FIGS. 62A-62B, then performing a reverse running stitch to fill any gaps between stitches, as shown in FIG. 62C. In some embodiments, the stitching results in 3 to 6 stitches along each flap. The stitching may be repeated on the second flap 831B, as shown in FIG. 62D. In some embodiments, the method may include performing a square knot with additional alternating knot at the bottom of each flap (e.g., a bottom of the opening 833, as shown in FIGS. 62E-62F. In some embodiments, the excess suture may be trimmed and cauterized to prevent fraying ends. In some embodiments, any or all of the stitching, reverse stitching, knotting, and cauterizing may be repeated for the second flap.

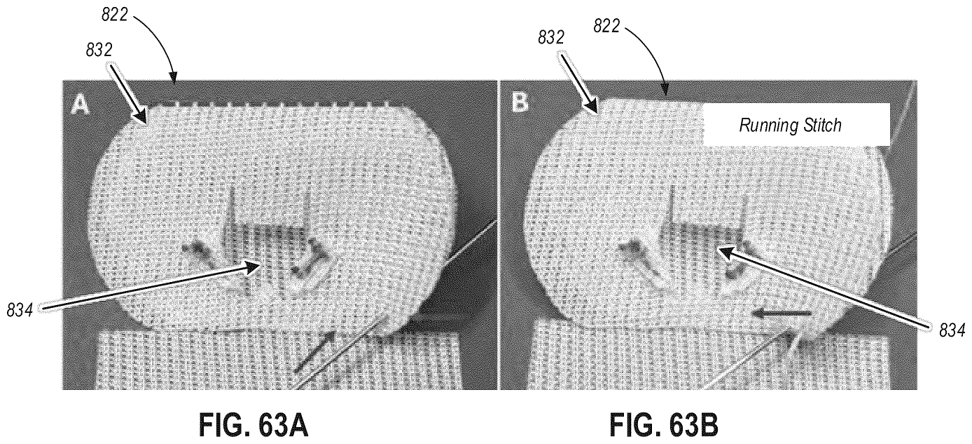

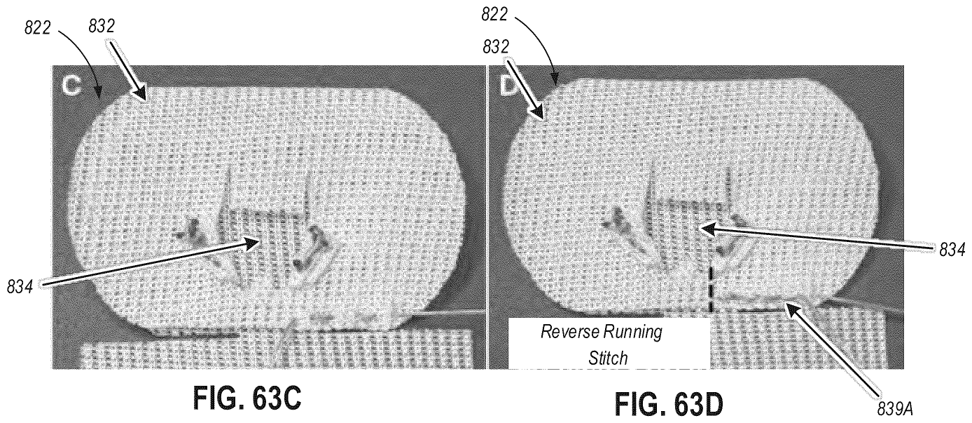

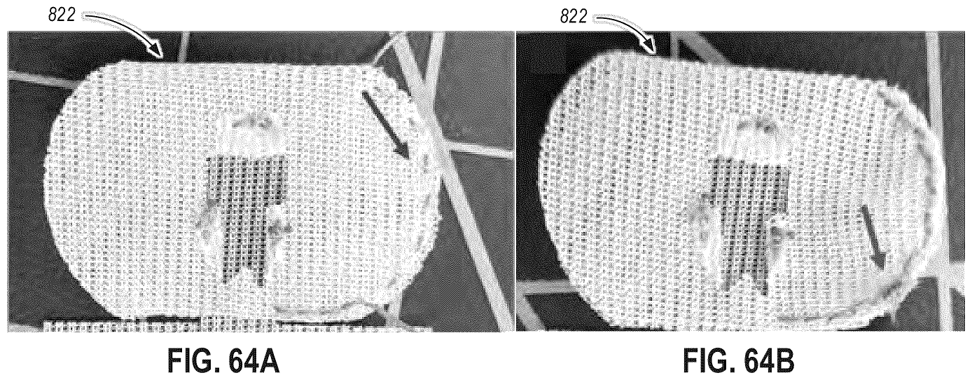

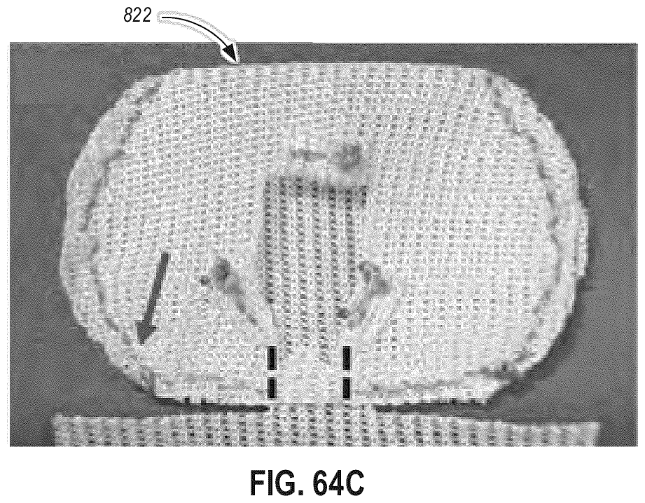

In some embodiments, the first portion of the fabric 832 may be folded over the second portion of fabric 834, as shown in FIGS. 63A-63D. For example, the fabric may be folded at the dotted line shown in FIG. 63A. In some embodiments, the suture knots from the flaps may face outward (e.g., away from the second portion 834 of fabric). In some embodiments, a running stitch may be performed at a corner of the first fabric (e.g., a lower right corner shown by the arrow in FIG. 63A). In some embodiments, the method may include leaving a tail with the suture (e.g., 1-2 inches) to tie a knot when the stitching is complete. In some embodiments, the running stitch may be continued toward a centerline of the fabric, as shown in FIG. 63C. In some embodiments, the running stitch may stop when in line with the edge of the flap, as shown in FIG. 63D. The method may include performing a reverse running stitch toward the starting point (e.g., the corner closest to the third portion of fabric). In some embodiments, a running stitch may be performed from the starting point toward a top corner of the fabric. In some embodiments, the stitch may follow a curvature of the fabric, as shown in FIG. 64A. In some embodiments, the method may include stitching over the folded and then reverse running stitch towards the starting point, as shown in FIG. 64B. In some embodiments, once the stitching is at the start point, the method may include tying a plurality of alternating knots. In some embodiments, the excess suture may be trimmed and cauterized. The steps of stitching the edge of the first portion and the second portion of fabric may be repeated for the second edge, as shown in FIG. 64C. The stitching may leave an opening or gap that aligns with the flaps, as indicated by the dotted lines in FIG. 64C.

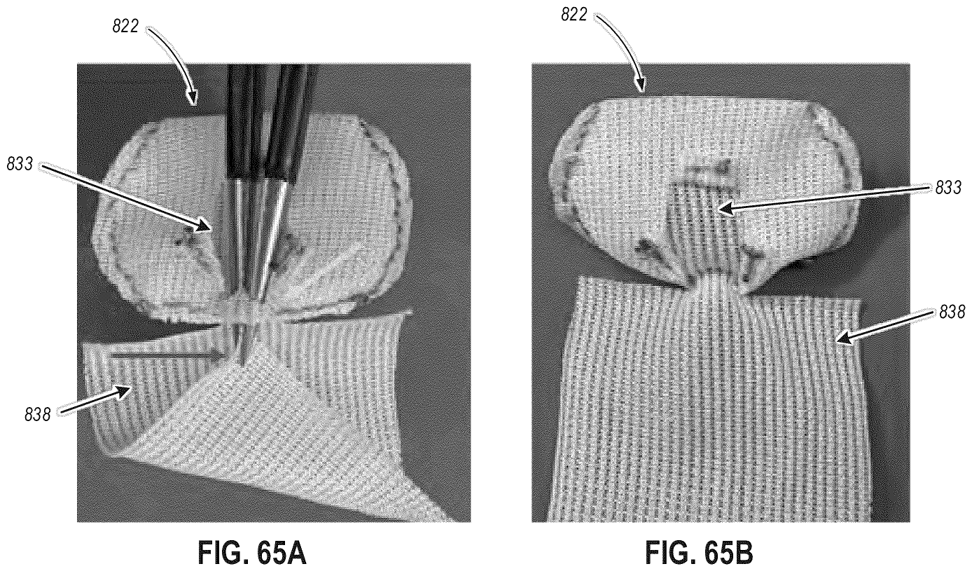

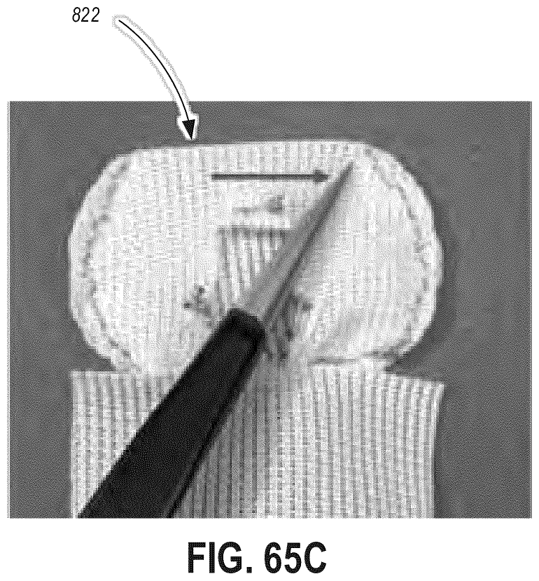

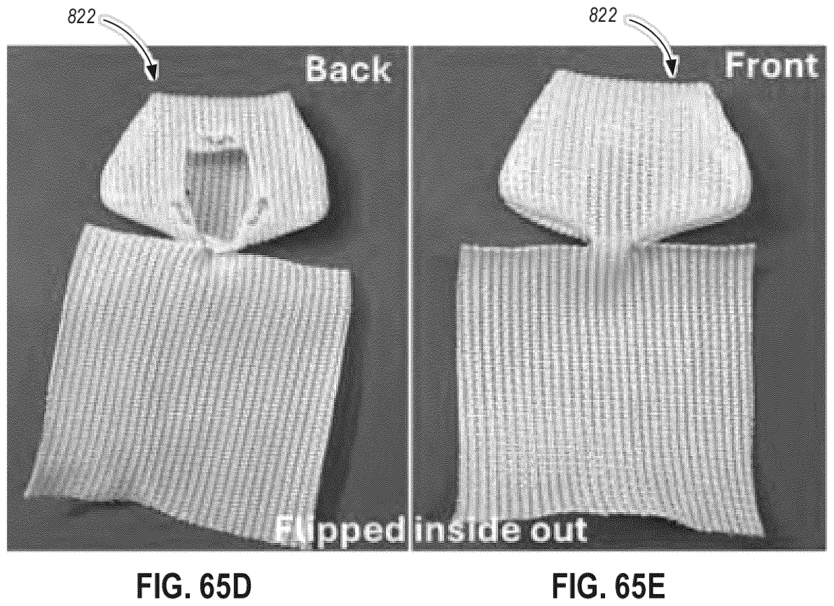

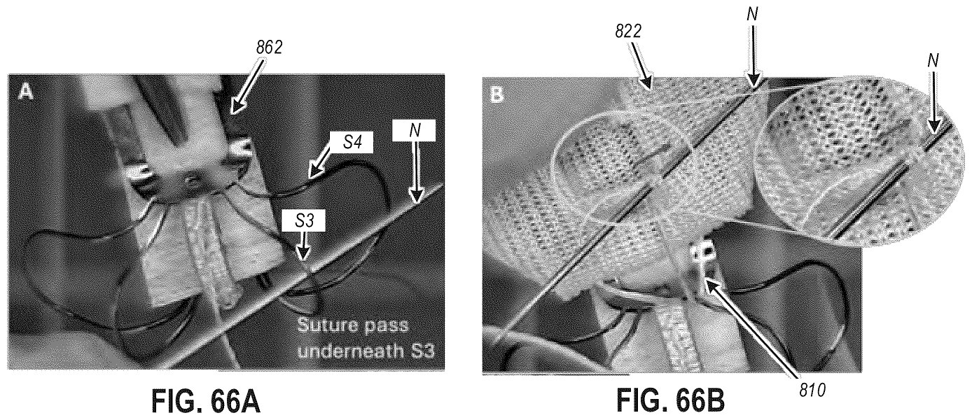

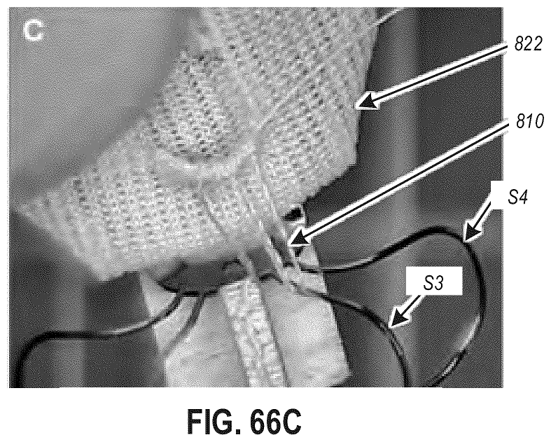

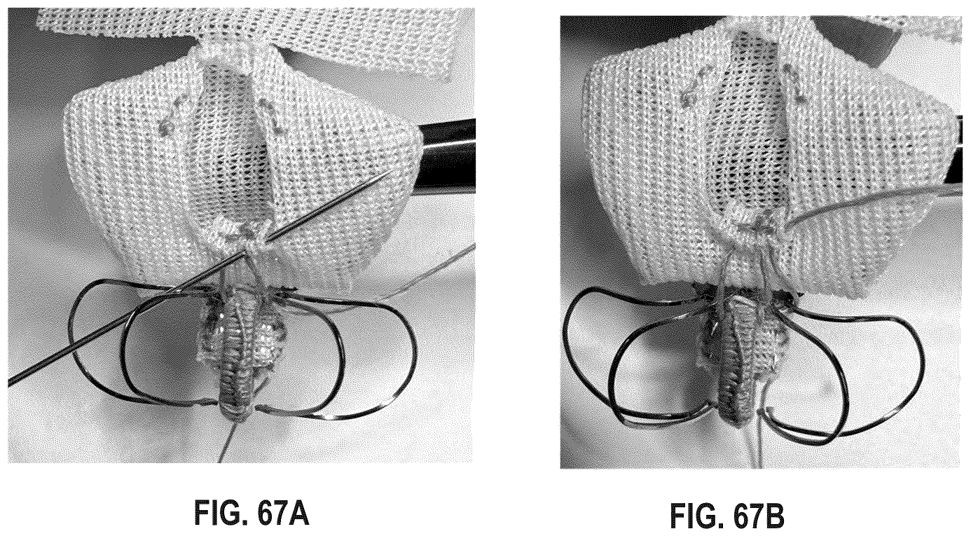

In some embodiments, the fabric may be flipped right side out, as shown in FIGS. 65A-65E. For example, forceps or tweezers can be used to grab one end of the third portion of fabric 838 and pull the third portion of fabric 838 through the opening 833 defined by the flaps. In some embodiments, the fabric of the first portion 832 and the second portion 834 may be flipped inside to form a pocket as shown in FIGS. 65C-65E such that the suture seams are inside the pocket. The pocket of the central cover 822 may be partially secured to the implant frame 860, as shown in FIGS. 66A-66C and FIGS. 67A-67B. With the back of the central cover 822 facing the operator, the suture can be passed into the center of the fold of the top seam in the central cover 822 in the direction indicated by the arrow shown in FIG. 60F. The method includes passing the suture (e.g., via needle N) underneath the third support member S3 and between the third support member S3 and the fourth support member S4, as shown in FIG. 66A. The method may include passing the needle N away from the base of the support member(s) such that the needle does not contact the implant frame (or any support members). The method can include passing the suture 810 into a corner of the seam near the opening of the central cover 822 in a direction indicated by the arrow in FIG. 66B-66C.

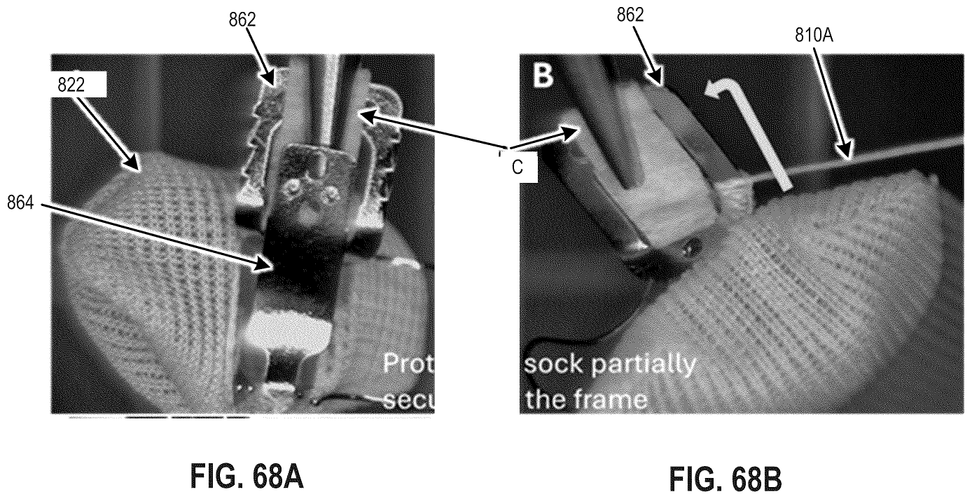

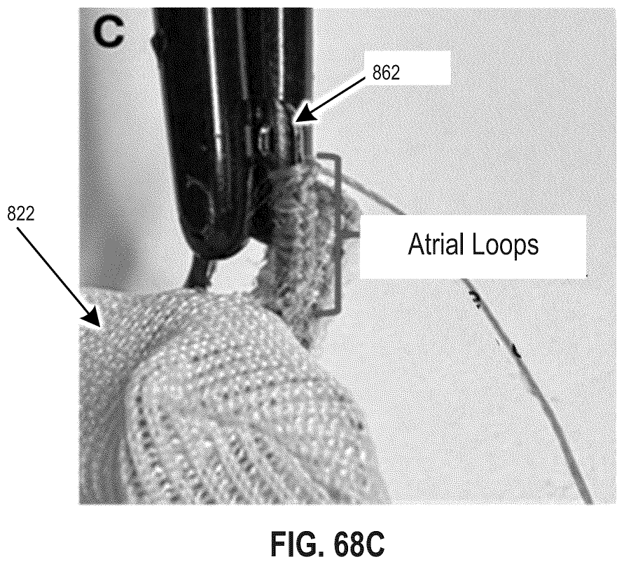

In some embodiments, the central cover 822 can be disposed on the implant frame by placing the pocket over the second, third, and fourth support members S2, S3, S4 and leaving the first support member S1 outside of the pocket of the central cover 822, as shown in FIGS. 68A-68B. This avoids deformation of the support members when the central cover 822 is mounted to the implant frame. Once the pocket is disposed over at least some of the support members S1-S4, the needle can be removed from the suture 810, and the suture 810 (e.g., the same suture) can be used to wrap the atrial arm.

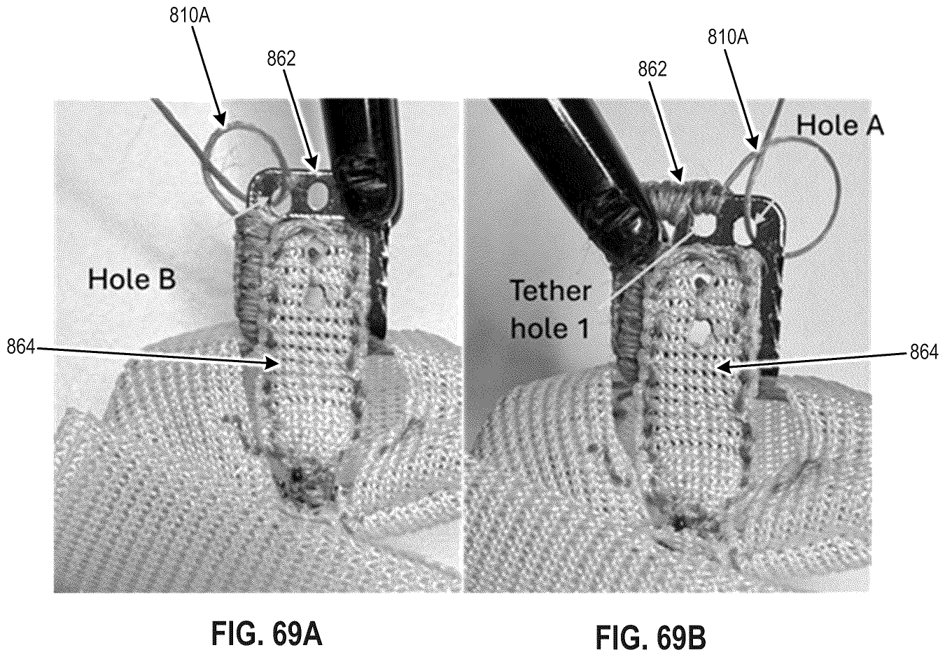

The method may include wrapping the atrial arm 862 from right to left (e.g., counterclockwise), as shown in FIGS. 69A-69B. For example, the first end 810A of the suture 810 may be disposed over a portion of the atrial arm 862 (e.g., from a front side of the implant toward a back side of the implant) to form a loop. The method may include using needle graspers and a lint free cloth & hemostat between the arms in the acceptable regions shown in FIG. 5, to help keep the atrial arm 862 and the ventricular arm 864 open while wrapping around the atrial arm. The atrial arm may be wrapped in a similar manner as described in FIGS. 8A-15B. The atrial arm 862 can be wrapped until the suture loops are adjacent to the first support member S1.

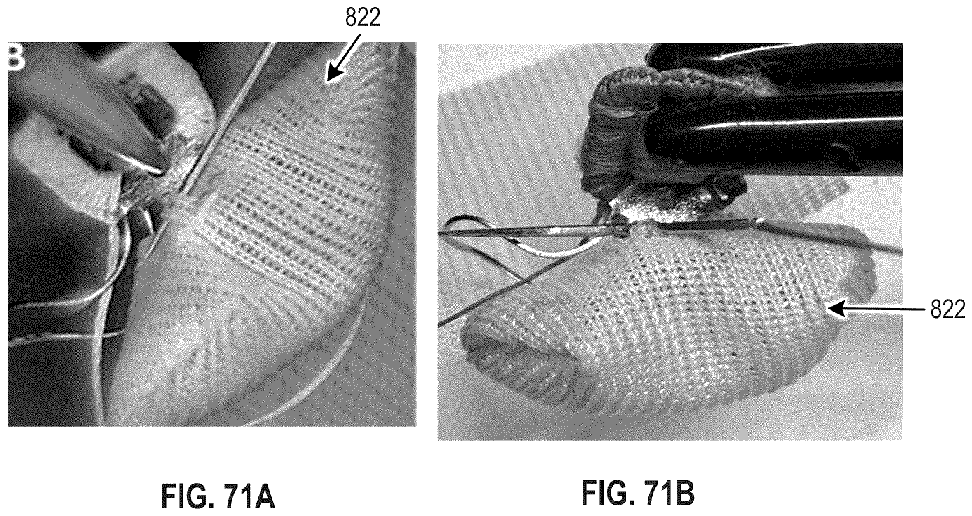

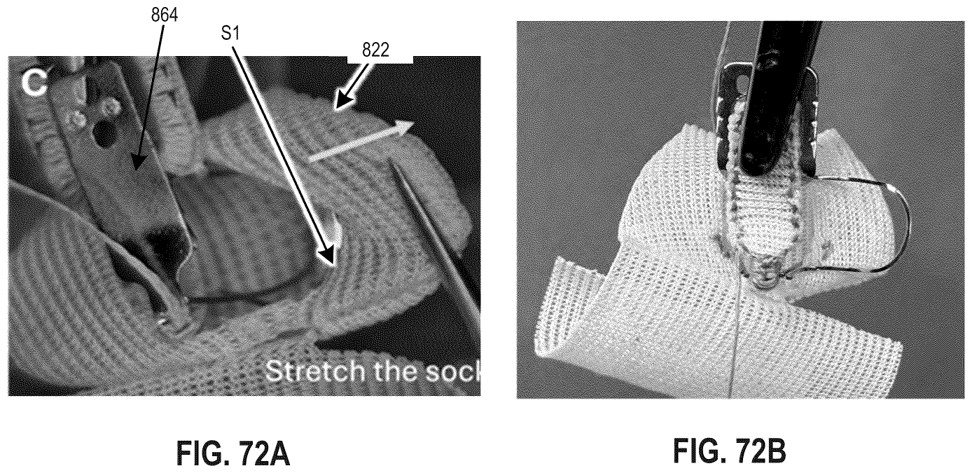

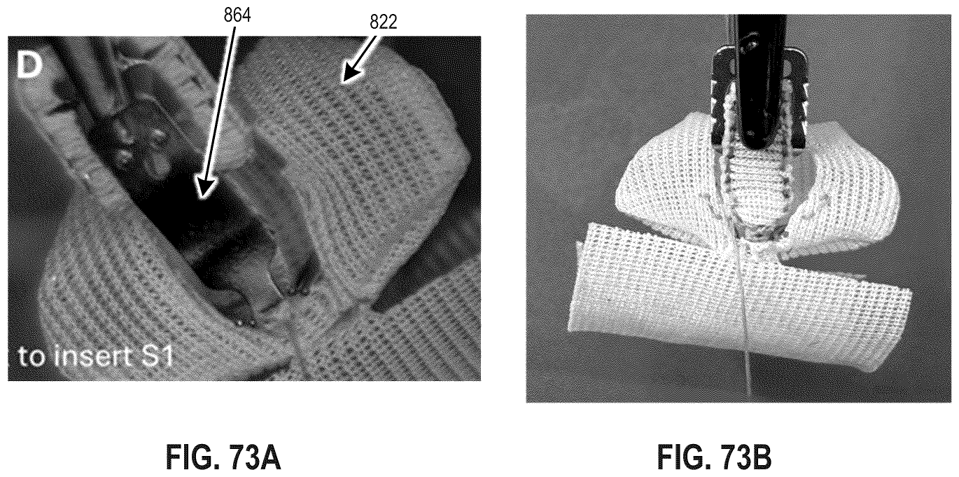

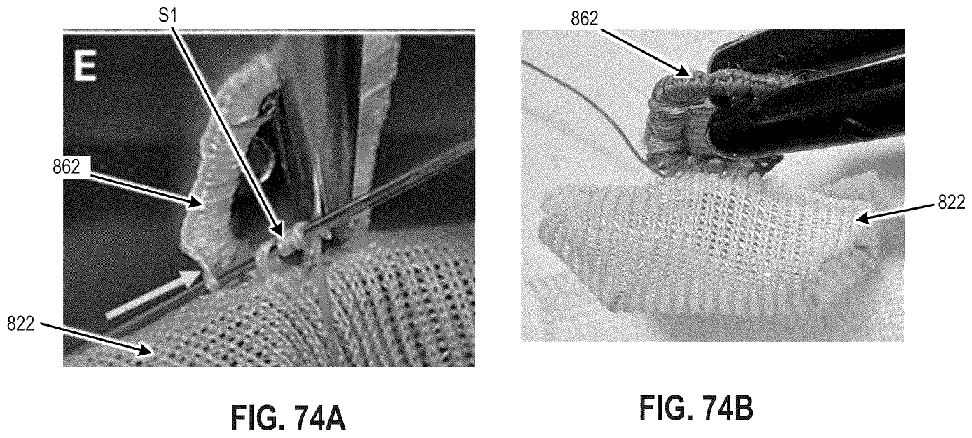

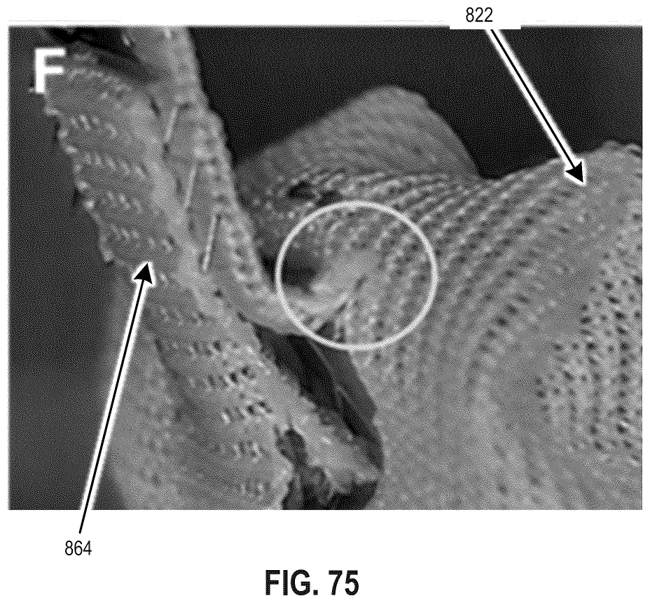

Once the suture loops are adjacent to the first support member S1, the central cover 822 can be fully secured to the implant frame, as shown in FIGS. 70A-75. The suture 810 can be passed underneath the first support member S1 between the first support member S1 and the second support member S2, as shown from different angles in FIG. 70A and FIG. 70B. As shown in FIGS. 71A and 71B, the method can include threading the first suture end 810 into a needle (e.g., size 10 or 12), and passing through the left corner of the seam (the same seam that the suture was passed through in FIG. 60F) in the direction as indicated by the arrow in FIG. 71A. As shown in FIGS. 72A-72B, a portion of the central cover 822 can be pulled away from a centerline of the implant (e.g., as shown by the arrow in FIG. 70C) such that the first support member S1 can be inserted in the pocket of the central cover 822, as shown in FIGS. 73A-73B. To secure the central cover 822 to the implant, the suture 810 can be passed into the atrial arm wrap (e.g., a portion of the suture 810 wrapped around the atrial arm 862) in the direction indicated by arrow in FIG. 74A and a knot (e.g., a cinch knot) can be performed. FIG. 74B shows a different angle of the central cover 822 secured to the implant frame with the support members disposed therein. The excess suture 810 can be trimmed and the knot can be cauterized, as shown in FIG. 75.

In some embodiments, once the pocket of the central cover 822 is secured to the implant frame, a pillow may be formed using the third portion of fabric 838 of the central cover 822 in a similar manner as described in FIGS. 46A-53. The pillow may act to extend the implant beyond its distal end, and therefore beyond an edge of the leaflet to which it is coupled, thereby improving coaptation with the opposing leaflet and increasing likelihood of closing the gap between the leaflets. In some embodiments, the shape of the pillow and/or the central cover can impact coaptation between the implant and the opposing leaflet. In some embodiments, a shape formed by the central cover (e.g., the central portion of the central cover or the pillow) can be modified or enhanced with additional material (e.g., additional fabric).

FIG. 76A shows a pattern of fabric for forming the central cover 922 (e.g., similar to the patterns shown in FIGS. 29 and 61). As shown, the fabric for forming the central cover 922 can include a first portion 932, a second portion 934, and a third portion 938. The first portion 932 and the second portion 934 can be configured to collectively form the pocket that surrounds the support members. The third portion 938 can be configured to be rolled to form the pillow 940 of the central cover 922. The third portion 938 can include extensions (e.g., wings, flaps, etc.) 938A, 938B of additional material that extend from lateral edges of the third portion 938. The extensions 938A, 938B can be configured to increase a surface area of the pillow 940 of the central cover 922 when the central cover is formed, as shown in FIG. 76B. The extensions 938A, 938B can be symmetrical to one another or asymmetrical to one another. As shown in FIG. 76B, when the fabric is folded into the central cover 922, the pillow 940 includes additional sections (shown in dashed line), thereby increasing a coaptation area of the implant.

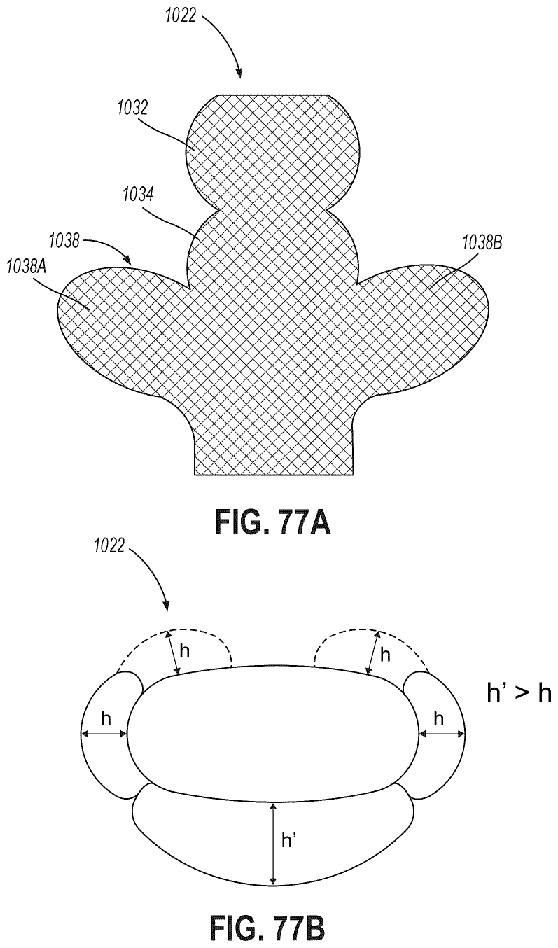

FIGS. 77A-77B show another embodiment of a pattern of fabric for forming a central cover 1022. As shown the fabric for forming the central cover 1022 can include a first portion 1032, a second portion 1034, and a third portion 1038. The first portion 1032 and the second portion 1034 can be configured to collectively form the pocket that surrounds the support members of the implant. The third portion 1038 can be configured to be rolled to form a pillow 1040. Due to the shape of the third portion 1038, the pillow 1040 can have a plurality of sections (e.g., petals, lips, etc.). The third portion 1038 can include extensions 1038A, 1038B extending from a portion (e.g., a top portion) of the lateral edges of the third portion 1038. Therefore, when the third portion 1038 is rolled to form the pillow 1040, the pillow 1040 can include the plurality of sections. In some embodiments, each section can have a height h, h1 (e.g., a dimension from an outer edge formed by the first and second portions 1032, 1034 and an outer edge of the respective section of the pillow 1040). In some embodiments, the sections of the pillow 1040 can have differing heights based on the shape of the pattern of the fabric. As shown, one section 1041 can have a larger height than the rest of the sections of the pillow 1040, thereby extending a distal portion of the implant beyond the support members such that the implant, when coupled to a leaflet, extends further beyond the edge of the leaflet. Therefore, the pillow 1040 may extend a longitudinal dimension of the implant.

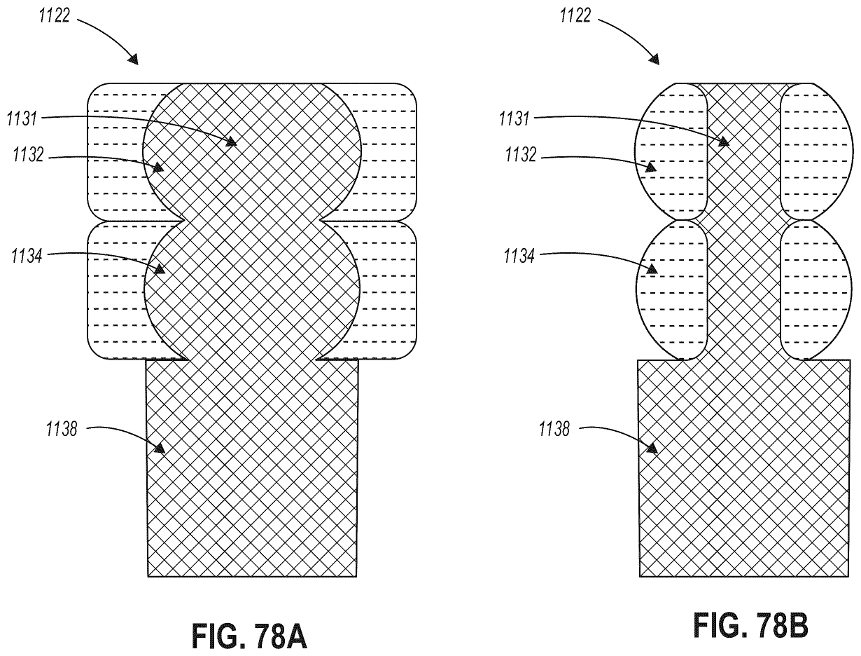

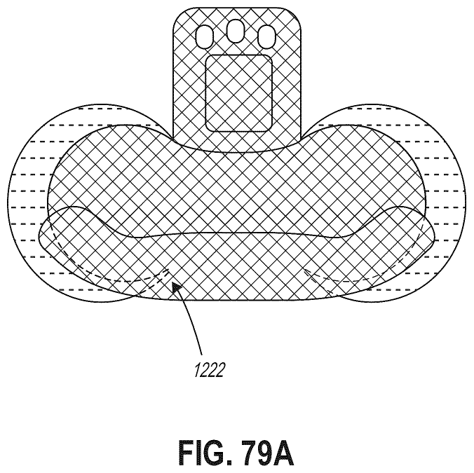

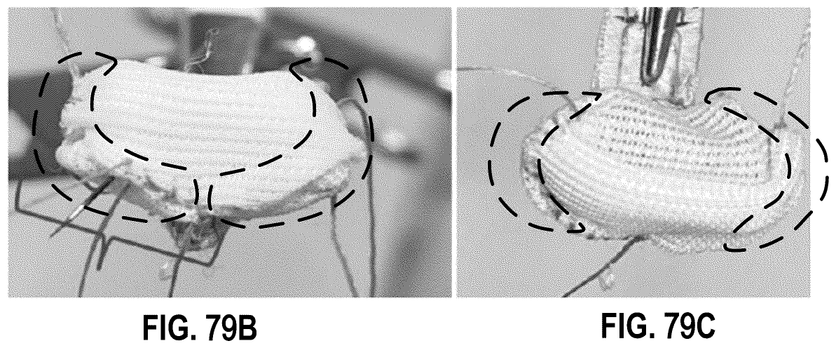

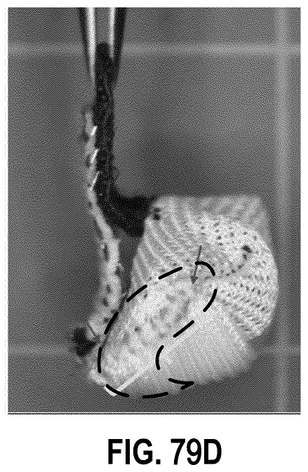

In some embodiments, a pocket of the central cover (e.g., the portion that holds the support members) can include extensions (e.g., wings, flaps, etc.) to adjust a shape of the coaptation section. FIG. 78A shows a pattern of fabric for forming a central cover 1122 including a first portion 1132, a second portion 1134, and a third portion 1138. The first portion 1132 and/or the second portion 1134 can include extensions that extend from lateral sides of the first portion and the second portion. As shown in FIG. 78B, when the first portion 1132 is folded over the second portion 1134 (e.g., as described with respect to FIGS. 32 and 63A), the extensions are folded inwards such that an outer perimeter of the first portion 1132 can be stitched to the second portion 1134 to form the pocket. When the pocket is flipped right side out (e.g., as described in FIGS. 35A-36C and 65A-65E, the extensions are disposed on the outside of the pocket and extend laterally outward. The extensions of the first portion can be secured (e.g., stitched) to the extensions of the second portion to form a single extension extending from each side of the central cover 1222, as shown in FIGS. 79A-79D. In some embodiments, the extensions or flaps can be moveable as the implant moves with the leaflet (e.g., similar to wings) to improve coaptation with the leaflet while reducing risk of damage to the leaflet. In some embodiments, the extensions or flaps can allow the implant to conform differently to an anterior leaflet versus a posterior leaflet.

In some embodiments, a shape of the central cover (e.g., the pillow or the pocket of the central cover) can be personalized to the patient based on the geometry of their heart valve leaflets. For example, if a patient has a larger gap between leaflets, a height of the pillow can be increased such that when the implant is coupled to the leaflet, the coaptation section of the implant extends further beyond the edge of the leaflet. In some embodiments, if the patient has an irregular gap between leaflets, the central cover 1222 can have extensions that have shapes corresponding to the shape of the gap between the leaflets. In some embodiments, the extensions can be easily deformable (e.g., such as the flaps shown in FIGS. 79A-79D) or have different sections (e.g., as shown in FIGS. 76B and 77B) such that the cover of the implant is malleable and can accommodate the geometry of the gap between the leaflets.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications and anatomy (e.g., intracranial and extracranial vascular structure) for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The specific examples and descriptions herein are exemplary in nature and embodiments may be developed by those skilled in the art based on the material taught herein without departing from the scope of the present invention.

1. An implant, comprising:
an implant frame configured to be disposed around a portion of a native leaflet of a heart valve of a patient;
a suture configured to be wrapped around a first portion of the implant frame to cover a surface of the first portion of the implant frame; and
a cover configured to be disposed around a second portion of the implant frame to form a coaptation surface of the implant, the coaptation surface configured to cause coaptation between the native leaflet and an opposing native leaflet in the heart valve.

2. The implant of claim 1, wherein the implant frame includes:
a first arm and a second arm configured to clamp the portion of the native leaflet therebetween; and
a central portion between the first arm and the second arm and configured to bias the first arm and the second arm towards one another.

3. The implant of claim 2, wherein the suture is configured to be wrapped around at least one of the central portion of the implant frame or the first arm.

4. The implant of claim 3, wherein the suture is configured to be wrapped around at least a majority of the central portion of the implant frame and the first arm.

5. The implant of claim 3, wherein a first portion of the cover is configured to be disposed over the central portion, and a second portion of the cover separate from the first portion is configured to be disposed over the second arm.

6. The implant of claim 5, wherein the implant frame includes one or more support members extending from at least one of the first arm or the central portion, the first portion of the cover configured to be disposed over the one or more support members to form the coaptation surface.

7. The implant of claim 6, wherein the cover further includes a pillow formed on an outer surface of the first portion of the cover and extending from the implant frame to increase an area of the coaptation surface formed by the first portion of the cover.

8. The implant of claim 1, wherein the cover includes a biocompatible fabric.

9. The implant of claim 1, wherein the cover is configured to be secured to the implant frame by a portion of the suture.

10. The implant of claim 1, wherein the implant further includes an opening in a portion of the implant frame configured to receive a tether configured to transition the implant between an open configuration and a closed configuration.

11. An implant, comprising:
an implant frame including:
a first end portion configured to be disposed around a portion of a native leaflet to couple the implant to a heart valve of a patient; and
a second end portion configured to extend into a gap of the heart valve; and
a plurality of support members extending from the first end portion of the implant frame to the second end portion of the implant frame; and
a cover configured to form a coaptation surface configured to contact an opposing native leaflet of the heart valve, a portion of the cover configured to be disposed around the second end portion of the implant frame, the cover further including a pillow formed on an outer surface of the portion of the cover and extending from the second end portion of the implant frame to increase an area of the coaptation surface formed by the portion of the cover.

12. The implant of claim 11, wherein the implant frame further includes:
a first arm and a second arm configured to clamp the portion of the native leaflet therebetween; and
a central portion between the first arm and the second arm and configured to bias the first arm and the second arm towards one another, the first arm and the second arm defining the first end portion of the implant frame, the central portion and the support members defining at least part of the second end portion of the implant frame.

13. The implant of claim 11, wherein the portion of the cover defines an inner volume configured to receive the second end portion of the implant, wherein the pillow is disposed outside the inner volume.

14. The implant of claim 13, further comprising:
a suture configured to be wrapped around a first portion of the implant frame to cover the first portion of the implant frame, wherein the suture is configured to secure the cover to the implant frame.

15. The implant of claim 14, wherein the suture is configured to wrap around the at least one of the central portion or the first arm such that a surface of the at least one of the central portion or the first arm of the implant frame is covered.

16. The implant of claim 14, wherein the portion of the cover is a first portion, the cover further including a second portion separate from the first portion and configured to be disposed around the second arm to cover the second arm.

17. The implant of claim 16, wherein the suture is configured to anchor the second portion of the cover to the implant frame to prevent sliding of the second portion of the cover relative to the implant frame.

18. The implant of claim 13, wherein the area of the coaptation surface corresponds to a size of the gap in the heart valve of a patient.

19. The implant of claim 13, wherein the cover includes flaps extending laterally from a centerline of the implant to increase a lateral dimension of a portion of the implant.

20. An implant, comprising:
an implant frame including:
a first end portion including a first arm and a second arm configured to clamp a portion of a native leaflet to couple the implant to a heart valve of a patient; and
a second end portion configured to extend into a gap of the heart valve, the second end portion including a central member coupled to the first arm and the second arm; and
a plurality of support members extending from the first end portion of the implant frame distally beyond the central member; and
a cover configured to be disposed around the plurality of support members to form a coaptation surface configured to contact an opposing native leaflet of the heart valve.

21. The implant of claim 20, wherein a portion of the cover is configured to be disposed around the plurality of support members, the cover further includes a pillow formed on an outer surface of the portion of the cover and extending from the second end portion of the implant to increase area of the coaptation surface formed by the portion of the cover.

22. The implant of claim 20, wherein the cover further includes flaps extending laterally from a centerline of the implant to increase a lateral dimension of a portion of the implant.

23. The implant of claim 20, wherein the implant further comprises a suture configured to be wrapped around a first portion of the implant frame to cover the first portion of the implant frame.

24. The implant of claim 20, wherein the cover includes a first portion configured to be disposed around the plurality of support members, the cover further including a second portion separate from the first portion and configured to be disposed around the second arm to cover the second arm.

25. The implant of claim 23, wherein the suture is configured to secure the cover to the implant frame.

26. The implant of claim 25, wherein the suture is configured to anchor the second portion of the cover to the implant frame to prevent sliding of the second portion of the cover relative to the implant frame.

27. The implant of claim 20, wherein the area of the coaptation surface corresponds to a size of the gap in the heart valve of a patient.

28. An implant, comprising:
an implant frame including:
a first arm and a second arm configured to clamp a portion of a native leaflet of a heart valve of a patient therebetween; and
a central portion between the first arm and the second arm;
a suture configured to be wrapped around a portion of the implant frame to cover a surface of the portion of the implant frame; and
a cover configured to be coupled to the implant frame, a first portion of the cover configured to be disposed around the central portion, a second portion of the cover separate from the first portion of the cover configured to be disposed around the second arm.

29. The implant of claim 28, wherein the second portion of the cover is formed with cauterized edges such that the second portion of the cover has a low profile.

30. The implant of claim 28, wherein the implant frame includes one or more support members, the first portion of the cover configured to be disposed over the central member and the one or more support members to form a coaptation surface configured to contact an opposing native leaflet of the heart valve.
